(19) United States
(12) Patent Application Publication
(10) Pub. No.: US 2025/0176509 A1
(43) Pub. Date: Jun. 5, 2025

(54) TELOMERASE REVERSE TRANSCRIPTASE THERAPY FOR KIDNEY FIBROSIS AND NON-HUMAN ANIMALS THEREOF

(71) Applicant: FUNDACIÓN DEL SECTOR PÚBLICO ESTATAL CENTRO NACIONAL DE INVESTIGACIONES ONCOLÓGICAS CARLOS III (F.S., Madrid (ES)

(72) Inventors: Maria Antonia BLASCO, Madrid (ES); Sarita SARASWATI, Madrid (ES); Paula MARTINEZ, Madrid (ES)

(21) Appl. No.: 18/832,416

(22) PCT Filed: Jan. 24, 2022
(86) PCT No.: PCT/EP2022/051505

§ 371 Date: Jul. 23, 2024

(51) Int. Cl. A01K 67/0276; C12N 9/12; C12N 15/90

(52) U.S. Cl. CPC A01K 67/0276; C12N 9/1276; C12N 15/907; C12Y 207/07049; A01K 2207/20; A01K 2217/075; A01K 2227/105; A01K 2267/03

(57) ABSTRACT

The invention provides compositions and methods useful for the treatment and prevention of conditions associated with short telomere length, particularly kidney fibrosis.

### FIELD OF INVENTION

This invention falls within the field of molecular biology, biotechnology and medicine. More particularly, it relates to non-human animals and to compositions and methods useful for the treatment of conditions associated with short telomere length. More particularly, it relates to compositions and methods useful for the treatment of conditions associated with kidney fibrosis associated to short telomere length.

### BACKGROUND OF THE INVENTION

Telomeres are specialized structures at the ends of chromosomes, which have a role in protecting the chromosome ends from DNA repair and degrading activities. Mammalian telomeres consist of TTAGGG repeats bound by a multi-protein complex known as shelterin. A minimum length of TTAGGG repeats and the integrity of the shelterin complex are necessary for telomere protection, as shortened telomeres have been associated with numerous diseases. These structures are essential for chromosome integrity by preventing telomere fusions and telomere fragility. Telomere length is controlled by the ribonucleoprotein enzyme telomerase which can de novo add telomeric sequences onto telomeres. Telomerase is a cellular reverse transcriptase (TERT, telomerase reverse transcriptase; also known as TP2; TRT; EST2; TCS1; hEST2) capable of compensating telomere attrition through de novo addition of TTAGGG repeats onto the chromosome ends by using an associated R A component as template (Terc, telomerase RNA component). Telomerase is expressed in most adult stem cell compartments; however, this is not sufficient to maintain telomere length as evidenced by the fact that telomere shortening occurs with age in most human and mouse tissues.

Because telomeric sequence is naturally lost upon every cell division (known as the end replication problem) and somatic cells express telomerase at very low levels or not at all telomeres shorten throughout life. When telomeres become critically short, they lose their protective function and a persistent DNA damage response at the telomeres is triggered which subsequently leads to a cellular senescence response (Harley et al., 1990, Flores et al., 2008).

Accumulation of short telomeres is a hallmark of aging. Mutations in telomerase or telomere-binding proteins lead to telomere shortening or dysfunction and are at the origin of human pathologies known as ‘telomere syndromes’, which are characterized by loss of the regenerative capacity of tissues and fibrotic pathologies and which include some cases of aplastic anemia, dyskeratosis congenita and fibrosis of the lung. Adeno-associated viruses (AAV) based telomerase gene therapy was found to be beneficial to extend health span, in the context of normal physiological aging in wild-type mice. Adult and aged mice were subjected to AAV9-mTERT gene therapy to broadly express the catalytic subunit of mouse telomerase (mTERT). The health span of the TERT treated mice was significantly increased, and aging was decelerated, as indicated by a number of physiological parameters (glucose and insulin tolerance, osteoporosis, neuromuscular coordination, rota-rod, etc). In addition, their mean lifespan, compared to control groups, was increased by 24% and 13% in adult an old mice, respectively. A single intravenous administration of AAV9-TERT in adult mice resulted in an increase in telomere length in peripheral blood cells (Bernardes de Jesus et al., 2012). These results highlight the importance of mouse models for the study of the specific pathologies and the potential therapeutic approaches.

Chronic kidney disease (CKD) is a disorder with high mortality, and its incidence is increasing owing to the demographic aging phenomenon. Kidney fibrosis is the primary determinant of end-stage renal disease characterized by fibroblast activation and excessive production and deposition of extracellular matrix (ECM), leading to destruction of renal parenchyma, an inflammatory and fibrotic response and decreased renal function. It is believed that short telomeres can contribute to kidney fibrosis. However, the role of short telomeres in kidney fibrosis remains less understood, in part due to the lack of appropriate mouse models. Further, it is not known whether telomerase-deficient mice with short telomeres will develop kidney fibrosis or they require additional insults to contribute to the disease. In the past, we have developed mouse models that develop aplastic anemia, and lung fibrosis associated with short telomeres. These mouse models demonstrate the role of short telomeres in the origin of these diseases and point to potential therapeutic strategies.

In the present invention, we develop suitable mouse model to study kidney fibrosis associated with short telomeres, which allows us to investigate whether telomerase activation can be an effective treatment to attenuate kidney fibrosis associated with short telomeres.

The invention provides compositions and methods useful for the treatment and prevention of kidney fibrosis associated with short telomeres.

### BRIEF DESCRIPTION OF THE FIGURES

FIG. 1: A mouse model of kidney fibrosis is associated with short telomeres. a, Schematic of the experimental approach. Increasing doses of FA (0, 50, 100 and 125 mg kg⁻¹ body weight (BW)) were administered at day 0 to either Tert^{+/+} or G3 Tert^{-/-} mice at 8-9 weeks of age. One and two weeks after FA administration, blood samples were taken for analysis. Mice were euthanized at day 14. b, Macroscopic appearance of kidneys at the end point of Tert^{+/+}, FA-treated Tert^{+/+}, G3 Tert^{-/-} and FA-treated G3 Tert^{-/-} mice. c,d, Blood creatinine (c) and BUN (d) levels in untreated and FA-treated Tert^{+/+} mice and G3 Tert^{-/-} mice. e, Representative images and quantification of Masson and PAS+D staining in Tert^{+/+}, FA-treated Tert^{+/+}, G3 Tert^{-/-} and FA-treated G3 Tert^{-/-} mice at the end point. Two-way analysis of variance (ANOVA) with post hoc Bonferroni's test was used for statistical analysis. Data are presented as mean values ±s.e.m. The number (n) of mice analyzed per genotype is indicated. *P≤0.05, **P≤0.01 and ***P≤0.001. NS, not significant.

FIG. 2: Severe kidney dysfunction in telomerase-deficient mice treated with a sublethal dose of folic acid. a, Schematic of the experimental approach. An FA dose of 125 mg kg⁻¹ of body weight was administered to Tert^{+/+} and G3 Tert^{-/-} mice at 8-9 weeks of age. b, Urinary albumin to creatinine ratio (UACR). c, Macroscopic appearance of kidneys in untreated and FA-treated Tert^{+/+} and G3 Tert^{-/-} mice. d, Representative images and quantification of Masson's trichrome, Sirius red and PAS+D stainings in untreated and FA-treated Tert^{+/+} kidneys and G3 Tert^{-/-} kidneys. One-way ANOVA with post hoc Tukey's test and two-way ANOVA with post hoc Bonferroni's test were used for statistical analysis. Data are presented as mean values±s.e.m. The number of mice analyzed per genotype is indicated. **P≤0.01 and ***P≤0.001.

FIG. 3: Telomerase-deficient mice show collagen deposition and activated myofibroblasts in the kidney after a sublethal dose of folic acid. a, Representative images and quantification of α-SMA, fibronectin, collagen type VI and double immunofluorescence for α-SMA and vimentin of Tert^{+/+}, FA-treated Tert^{+/+}, G3 Tert^{-/-} and FA-treated G3 Tert^{-/-} mice. b, Double immunofluorescence for α-SMA and Ki67 of Tert^{+/+}, FA-treated Tert^{+/+}, G3 Tert^{-/-} and FA-treated G3 Tert^{-/-} mice. White arrows indicate α-SMA⁺Ki67⁺ cells. c, Relative mRNA expression of Acta2, Vim, Col1a1, Co/3a1, Co/4a1 and Fn1 in Tert^{+/+}, FA-treated Tert^{+/+}, G3 Tert^{-/-} and FA-treated G3 Tert^{-/-} mice at 14 d after FA administration. One-way ANOVA with post hoc Tukey's test was used for statistical analysis. Data are presented as mean values ±s.e.m. The number of mice analyzed per genotype is indicated. **P≤0.01 and ***P≤0.001. Vh, vehicle.

FIG. 4: Increased kidney apoptosis and senescence in telomerase-deficient mice after a sublethal dose of folic acid. a-d, Representative images and quantification of immunohistochemistry stainings of the CC3 (a), p21 (b), p53 (c) and γ-H2AX (d) in Tert^{+/+}, FA-treated Tert^{+/+}, G3 Tert^{-/-} and FA-treated G3 Tert^{-/-} mice. Insets: amplified images. e, Representative images and quantification of mean total nuclear telomere length of Q-FISH analysis in Tert^{+/+}, FA-treated Tert^{+/+}, G3 Tert^{-/-} and FA-treated G3 Tert^{-/-} mice. Amplified images are shown below. a.u.f., arbitrary units of fluorescence. Statistical significance was determined by one-way ANOVA with post hoc Tukey's test. *P≤0.05, **P≤0.01 and ***P≤0.001. Data are presented as mean values ±s.e.m. The number of mice analyzed per genotype is indicated.

FIG. 5: Short telomeres induce tubular damage and immune infiltration in kidney. a, Relative expression of Havcr1, Lcn2 and Emr1 in Tert^{+/+}, FA-treated Tert^{+/+}, G3 Tert^{-/-} and FA-treated G3 Tert^{-/-} mice 14 d after administration of low-dose FA. b, Representative images and quantification of F4/80, CD3e, CD4 and CD8a immunohistochemistry staining. Insets: amplified images. One-way ANOVA with post hoc Tukey's test was used for statistical analysis. Data are presented as mean values±s.e.m. The number of mice analyzed per genotype is indicated. **P≤0.01 and ***P≤0.001.

FIG. 6: Hyperactivation of EMT and TGFβ-signaling pathways in telomerase-deficient mice. a,b, Gene expression data obtained by RNA-seq of kidney samples from 10-week-old Terc^{+/+} and G3 Tert^{-/-} mice untreated or treated with FA at a dose of 125 mg kg⁻¹ body weight. Mice were euthanized 14 d after treatment. Samples were analyzed by GSEA to determine significantly enriched gene sets. GSEA plots comparing EMT (a) and TGFβ-signaling pathways (b) for untreated Tert^{+/+} versus untreated G3 Tert^{-/-} mice, FA-treated Tert^{+/+} versus FA-treated G3 Tert^{-/-} mice, FA-treated Terc^{+/+} versus untreated Tert^{+/+} mice, and FA-treated G3 Tert^{-/-} versus untreated G3 Tert^{-/-} mice. The red to blue horizontal bar represents the ranked list. Genes located at the central area of the bar show small differences in gene expression between the pairwise comparisons. At the red edge of the bar are genes showing higher expression levels, and at the blue edge of the bar are genes showing lower expression levels. Red and blue arrows indicate upregulation and downregulation, respectively, of the pathway in the pairwise comparisons. False discovery rates (FDR) are indicated. Samples correspond to kidneys of four independent Tert^{+/+}, G3 Tert^{-/-} and FA-treated G3 Tert^{-/-} mice and three FA-treated Tert^{+/+} mice. c, Relative expression of Tgfb1, Snail1, Snail2, Twist1, Zeb1, Zeb2, Loxl2, Cdh1 and Smad3 in Tert^{+/+}, FA-treated Tert^{+/+}, G3 Tert^{-/-} and FA-treated G3 Tert^{-/-} mice at 14 d after administration of a sublethal FA dose. d, Representative images and quantification of E-cadherin and phosphorylated SMAD3 (p-SMAD3) immunohistochemistry stainings. Insets: amplified p-SMAD3 staining images. One-way ANOVA with post hoc Tukey's test was used for statistical analysis. Data are presented as mean values±s.e.m. The number of mice analyzed per genotype is indicated. *P≤0.05, **P≤0.01 and ***P≤0.001.

FIG. 7: Trf1 deletion induces kidney fibrosis. a, Schematic of the experimental approach. b, Representative images and quantification of Masson's trichrome, Sirius red and SMA stainings in Trf1^{+/+} and Trf1^{flox/flox} mice. c, Relative expression of Trf1 and mesenchymal markers Acta2 and Fn1 in Trf1^{+/+} and Trf1^{flox/flox} mice. d, Relative expression of EMT markers Tgfb1, Snail1, Snail2, Twist1, Zeb1 and Zeb2 in Trf1^{+/+} and Trf1^{flox/flox} mice. A two-tailed t-test was used for statistical analysis. Data are presented as mean values ±s.e.m. The number of mice analyzed per genotype is indicated. *P≤0.05, **P≤0.01 and ***P≤0.001.

FIG. 8: TERT activation rescued the EMT phenotype in vitro. a,b, Relative mRNA mTERT expression (a) and telomere length analysis in wild-type and G3 Tert^{-/-} PTCs (b) transduced either with the empty (null) or mTert-containing vector. c, Immunofluorescence for E-cadherin, SMA and Snail1/Slug in PTCs from 10- to 11-week-old Tert^{+/+} and G3 Tert^{-/-} PTCs transduced either with the empty (null) or mTert-containing vector. Twenty micrographs were captured for each condition. d, Relative mRNA Cdh1, Acta2, Vim, Col3a1, Col4a1, Tgfb1, Snail1, Snail2 and Zeb1 expression. Data are presented as mean values±s.e.m. The number of mice analyzed per genotype is indicated. One-way ANOVA post hoc Tukey's test was used for statistical analysis. *P≤0.05, **P≤0.01 and ***P≤0.001.

FIG. 9: Telomerase-deficient mice with short telomeres do not spontaneously develop kidney fibrosis. a, Scheme of the experimental approach. b-f, Representative images and quantification of Masson trichrome (b), Sirius Red (c), PAS-Diastase (d), SMA (e) and E-cadherin (f) in Tert^{+/+} and G3Tert^{-/-} mice. g-h, Representative images and quantification of p21 (g) and CC3 (h) immunohistochemistry stainings in Tert^{+/+} and G3Tert^{-/-} mice. Insets show amplified images. A t-test two tailed was used for statistical analysis. Data is presented as mean values+/−SEM. The number of mice analyzed per genotype is indicated.

FIG. 10: Blood parameters. Blood creatinine (a) and blood urea nitrogen (BUN) (b) levels of untreated and FA treated Tert^{+/+} and G3Tert^{-/-} mice. Blood samples were collected at days 2, 7 and 14. Mice were sacrificed at day 14. a, Two-way Anova with post hoc Bonferroni test was used for statistical analysis. Data is presented as mean values+/−SEM. The number of mice analyzed per genotype is indicated. *p≤0.05; **p≤0.01; ***p≤0.001.

FIG. 11: Effect of short telomeres on cell cycle regulators. Relative expression of CCnd1, CCnd2, CCnb1 and CCne1 in Tert^{+/+}, FA-treated Tert^{+/+}, G3Tert^{-/-} and FA-treated G3Tert^{-/-} mice 14 days after administration of a low dose of FA. One-way Anova was used for statistical analysis. Data is presented as mean values+/−SEM. The number of mice analyzed per genotype is indicated. *p≤0.05; **p≤0.01; ***p≤0.001.

FIG. 12: Effect of shorter telomeres on EMT related genes. a, Gene expression data obtained by RNA-seq of kidney samples from 7- and 47-week old Terc^{+/+} and G3 Tert^{-/-} mice. b, Gene Set Enrichment Analysis (GSEA) plots for the EMT pathway. False Discovery Rates (FDR) are indicated. Samples correspond to kidneys of five independent Tert^{+/+}, G3 Tert^{-/-} mice.

FIG. 13: Effect of shorter telomeres on nephric-progenitor genes. a, Relative expression of Sox-9, Wt-1, Pax-2, Sall2, Acvr2b, Klotho in in Tert^{+/+}, FA-treated Tert^{-/-}, G3Tert^{-/-} and FA-treated G3Tert^{-/-} mice 14 days after administration of a low dose of FA. b, Representative images and quantification of Sox-9 immunohistochemistry staining. The insets show amplified images. One-way Anova was used for statistical analysis. One-way Anova was used for statistical analysis. Data is presented as mean values+/−SEM. The number of mice analyzed per genotype is indicated. *p≤0.05; **p≤0.01; ***p≤0.001.

FIG. 14: Effect of shorter telomeres on Notch-targeted genes. Relative expression of Notch1, 2, 3, Jagged1 and Tfam in Tert^{+/+}, FA-treated Tert^{+/+}, G3Tert^{-/-} and FA-treated G3Tert^{-/-} mice 14 days after administration of a low dose of FA. One-way Anova was used for statistical analysis. Data is presented as mean values+/−SEM. The number of mice analyzed per genotype is indicated. *p≤0.05; **p≤0.01; ***p≤0.001.

FIG. 15: TERT activation rescued the EMT phenotype in vitro. a,b, Microscopic bright field images of proximal tubule cell (PTC) culture at day 8 (a) and day 14 post-transduction with either the empty or the mTert containing vector (b). c, Representative images of Immunofluorescence for E-Cadherin, SMA and Tgfβ1. Twenty micrographs were captured from each condition.

### BRIEF DESCRIPTION OF THE INVENTION

In one aspect, the present invention provides a nucleic acid vector comprising a coding sequence for telomerase reverse transcriptase (TERT) for use in treating kidney fibrosis associated to the presence of short telomeres. Preferably, TERT is encoded by a nucleic acid sequence comprising the sequence of SEQ ID NO: 1 or SEQ ID NO: 3. Preferably, TERT is encoded by a nucleic acid sequence consisting of the sequence of SEQ ID NO: 1 or SEQ ID NO: 3. Preferably, TERT comprises an amino acid sequence of SEQ ID NO: 2 or SEQ ID NO: 4. Preferably, TERT consists of an amino acid sequence of SEQ ID NO: 2 or SEQ ID NO: 4. Preferably, the nucleic acid sequence encoding TERT is operably linked to a regulatory sequence that drives the expression of the coding sequence. Preferably, the vector is a non-integrative vector. Preferably, the vector is a ribonucleic acid (RNA), preferably a messenger RNA.

Preferably, wherein the vector is an adeno-associated virus-based non-integrative vector. Preferably, the vector is an adeno-associated virus-based vector derived from a serotype 9 adeno-associated virus (AAV9). Preferably, the capsid of the adeno-associated virus-based vector is made of capsid proteins of the serotype 9 adeno-associated virus (AAV9), and the nucleic acid sequence contained in the capsid is flanked at both ends by internal terminal repeats corresponding to serotype 2 adeno associated viruses. Preferably, the nucleic acid contained in the capsid comprises a fragment which encodes the amino acid sequence coding for TERT. Preferably, the vector comprises a regulatory sequence which is a constitutive promoter, preferably the cytomegalovirus (CMV) promoter.

In a further aspect, the present invention provides a A non-human animal characterized in that it exhibits a pathological condition of kidney fibrosis, wherein the non-human animal is obtained or obtainable when a sublethal dose of folic acid is administered to a non-human animal whose germ cells comprise a hereditary deactivation of both alleles of the Tert gene. Preferably, the animal is a mammal, preferably a rodent. The sublethal dose of folic acid is preferably a dose of up to 200 mg/kg body weight, preferably of 125 mg/kg body weight.

Preferably, the folic acid is administered intraperitoneally. Preferably, the folic acid is administered when the animals are between 4-10 weeks of age, preferably between 6-8 weeks of age. Preferably, wherein the folic acid is administered once.

Preferably, the non-human animal whose germ cells comprise a hereditary deactivation of both alleles of the Tert gene (Tert−/−) is the third generation (G3) of Tert−/− lineage.

### DETAILED DESCRIPTION OF THE INVENTION

The role of short telomeres in aplastic anemia and lung fibrosis was found to be important mainly due to the development of appropriate mouse models, which opened the path for the development of potential therapeutic strategies against said diseases. However, other complex diseases, such as kidney fibrosis, lack of suitable animal models, hampering the study of the role of short telomeres in said pathologies.

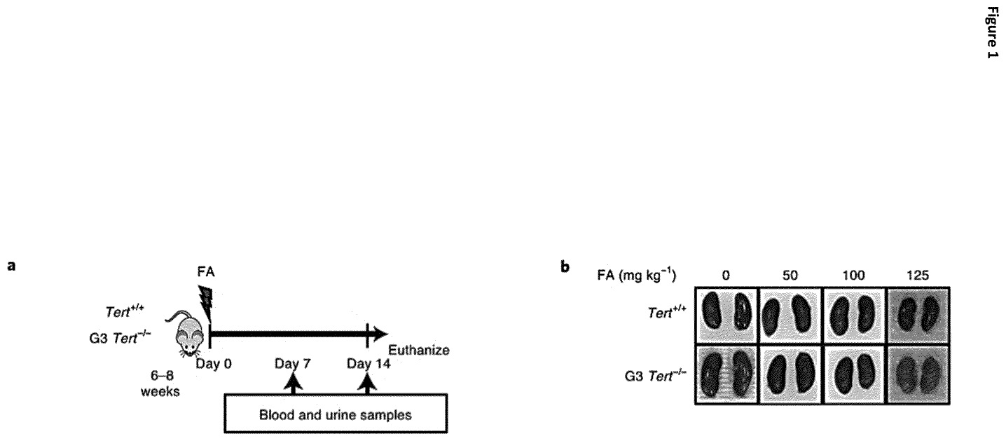

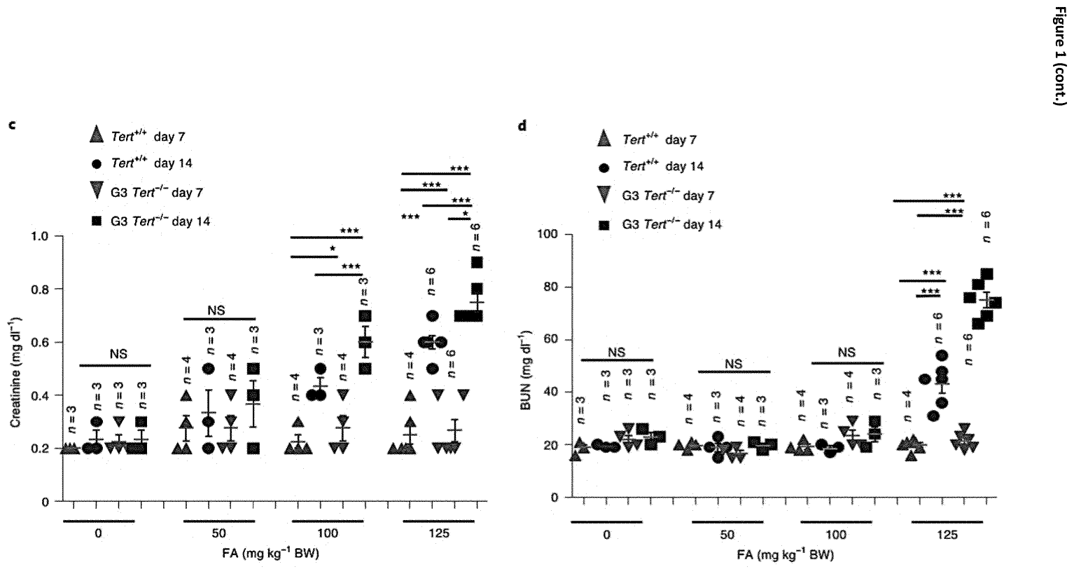

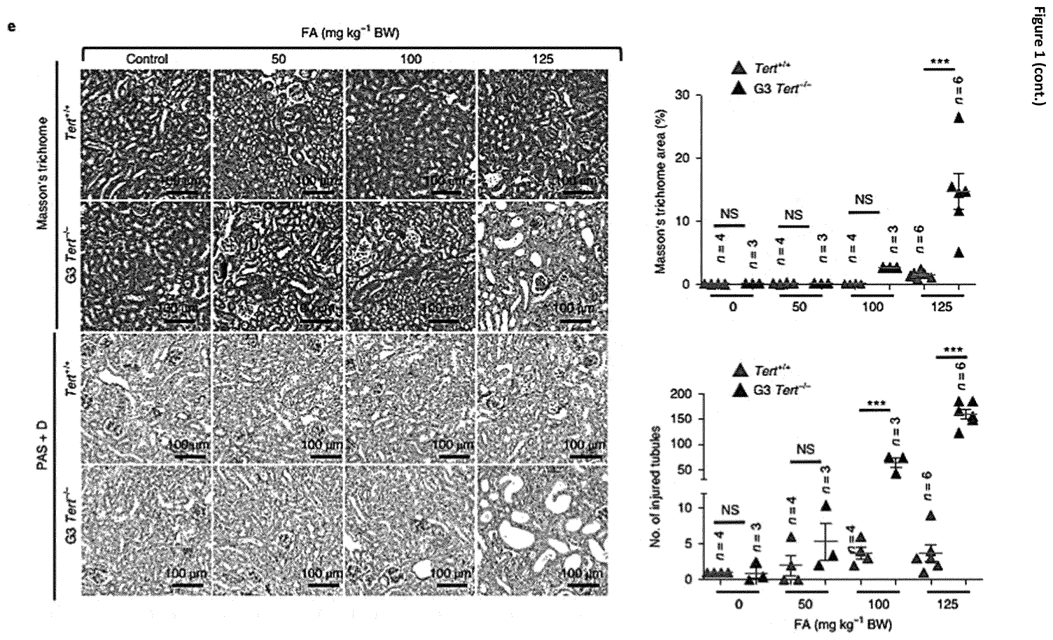

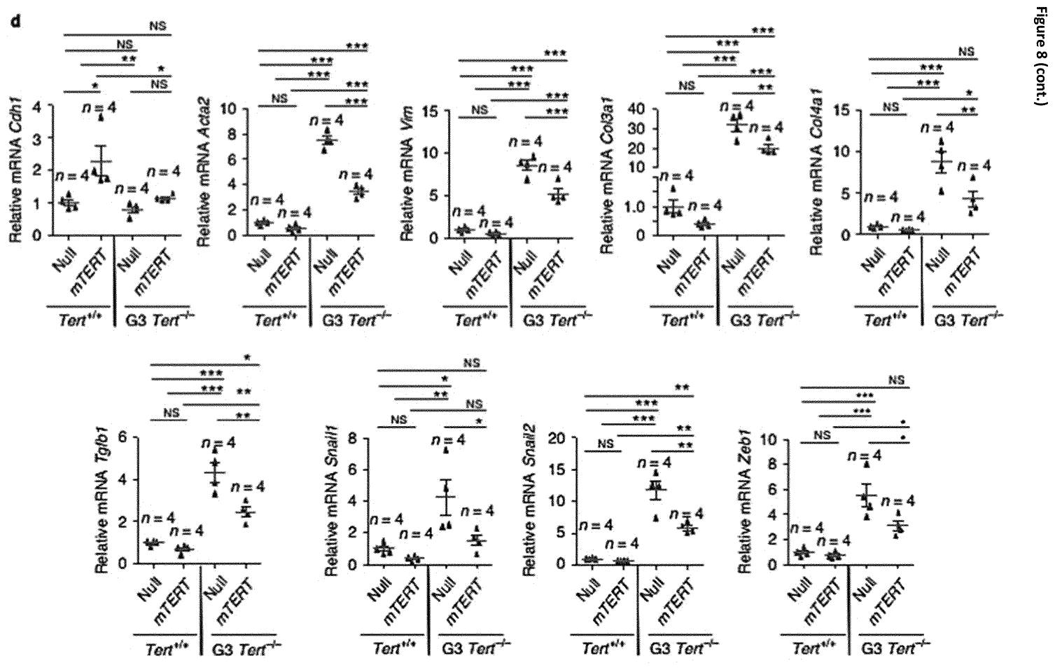

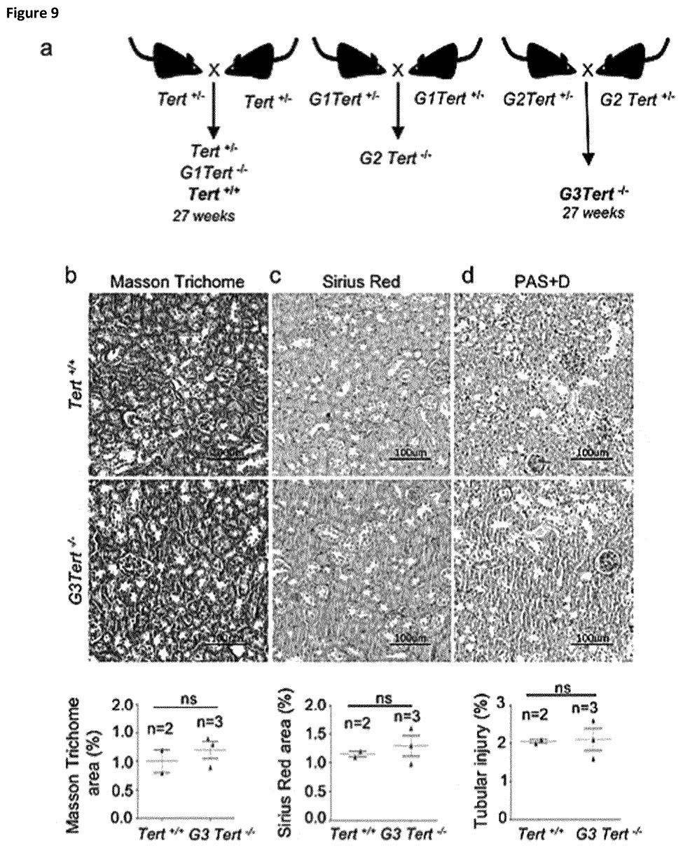

In the present study, we aimed at studying the involvement of short telomeres in kidney fibrosis. In order to do that, suitable animals models had to be developed. We first analysed the kidneys of wild-type mice, as well as mice lacking the telomerase catalytic subunit, Tert, that were bred for three generations (G3) and thus are G3 Tert^{-/-} mice (FIG. 9a). However, we found that both wild-type (Tert^{+/+}) and G3 Tert^{-/-} mice aged 8-9 weeks displayed grossly normal kidney histology, with no evidence of glomerular or tubular defects and with no fibrosis or accumulation of collagen fibers (FIG. 9b, c). Similarly, mice lacking the telomerase catalytic subunit Tert did not have an increased tubular injury or loss of brush borders in the kidney. Also, in agreement with normal kidneys in the G3 Tert^{-/-} mice, no differences were found in the presence of activated fibroblasts, in apoptosis, in senescence, or in the expression of E-cadherin (a marker of EMT, associated with tissue fibrosis), compared with age-matched Tert^{+/+} mice (FIG. 1e-h). These results led us to the conclusion that telomerase-deficient mice per se do not spontaneously develop kidney fibrosis, raising the question whether mice with short telomeres require of additional factors to contribute to the development of this pathology. In this regard, since the incidence of kidney fibrosis increases with age, we also questioned whether kidney fibrosis is likely to be caused by the combination of molecular and cellular aging events, such as the presence of short telomeres together with exogenous damage to the kidney.

Folic acid (FA) induces interstitial fibrosis, but only at high doses. High dosages of folic acid (250 ug/g BW) given i.p in mice induces folic acid crystals rapidly with tubular necrosis in the acute phase (1-14 days) and patchy interstitial fibrosis in the chronic phase (28-42 days). However, lower doses of FA were not found to induce kidney fibrosis in wild-type mice. In view of this, we aimed to study the effect of a sublethal dose of FA in Tert^{+/+} and G3 Tert^{-/-} mice aged 8-9 weeks, and its contribution to the development of kidney fibrosis associated to short telomeres. To this end, we first subjected Tert^{+/+} and G3 Tert^{-/-} mice aged 8-9 weeks to increasing doses of FA (50, 100, 125 and 250 mg kg-1 body weight; FIG. 1a) and selected the highest dose of FA that did not induce kidney fibrosis in wild-type mice. The results showed that 125 mg kg-1 was the maximum tolerated dose of FA that did not cause lethality in wild-type mice with normal telomere length (FIG. 1). We next studied whether that this dose of FA, although not sufficient to induce kidney fibrosis in Tert^{+/+} mice, may synergize with short telomeres in telomerase-deficient aged mice to induce kidney fibrosis. After carrying out a series of test, we found that telomerase-deficient mice treated with a sublethal dose of FA show all the hallmarks of the human disease including severe kidney dysfunction, as indicated by elevated blood levels of creatinine and urea.

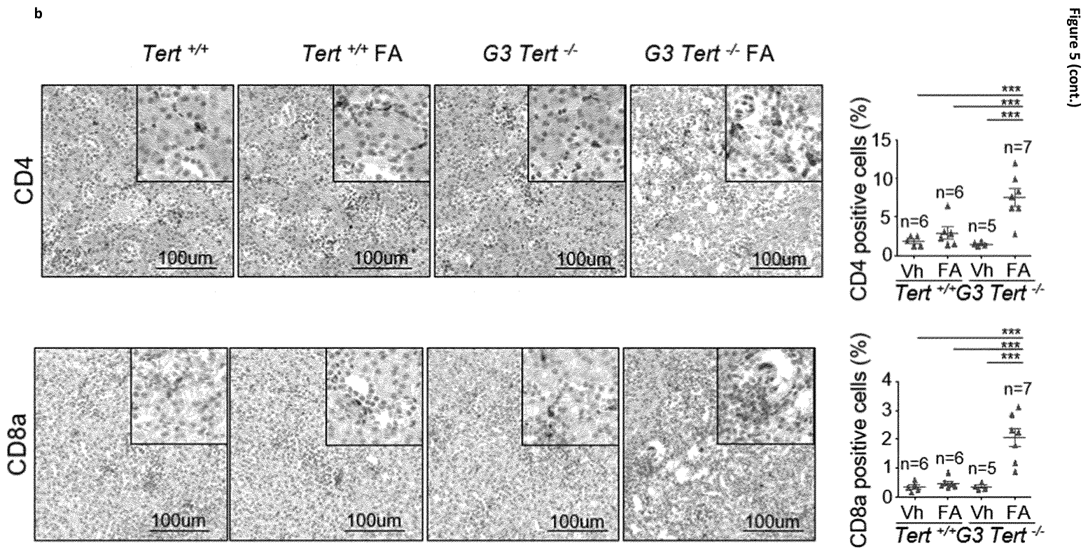

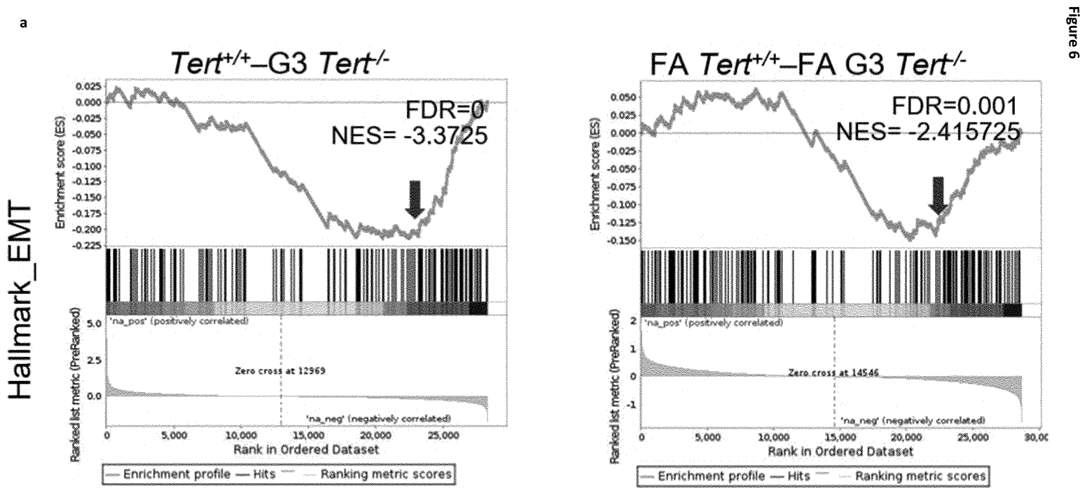

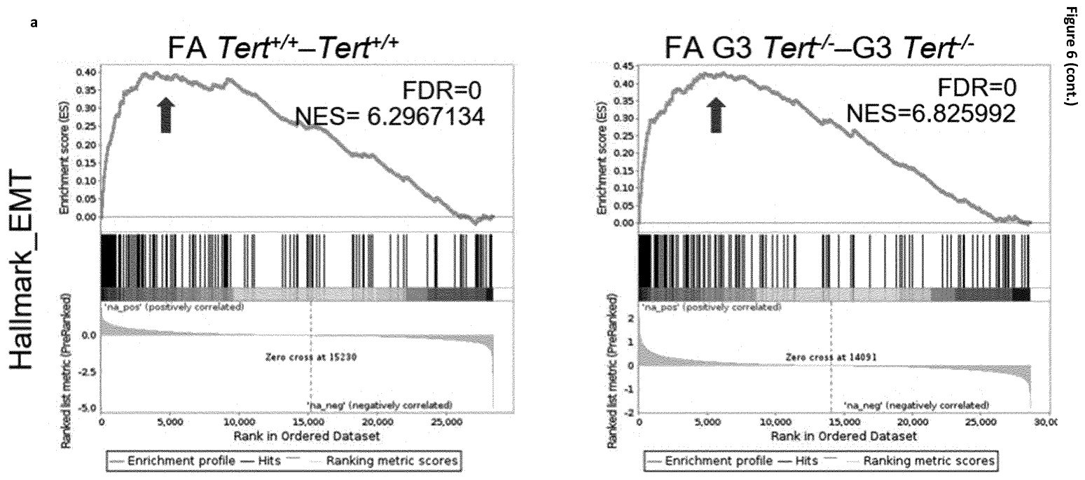

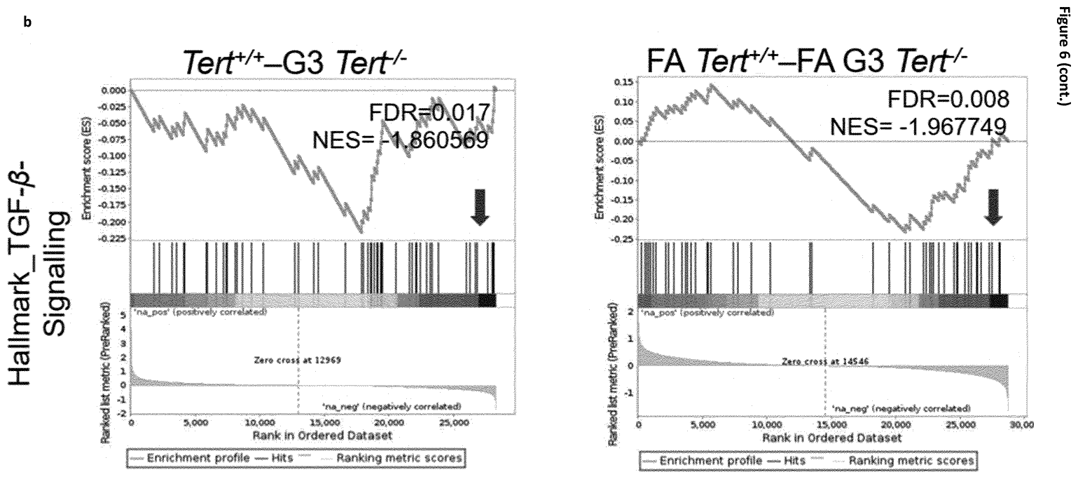

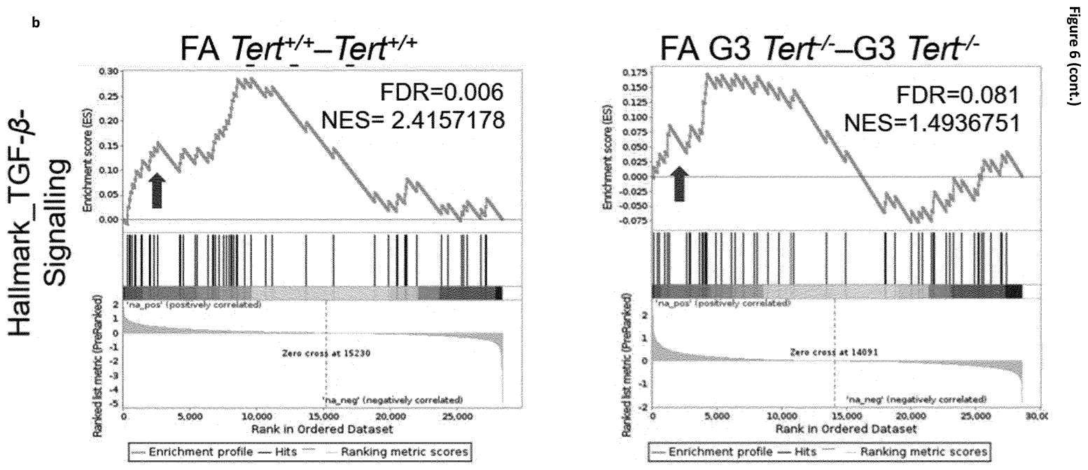

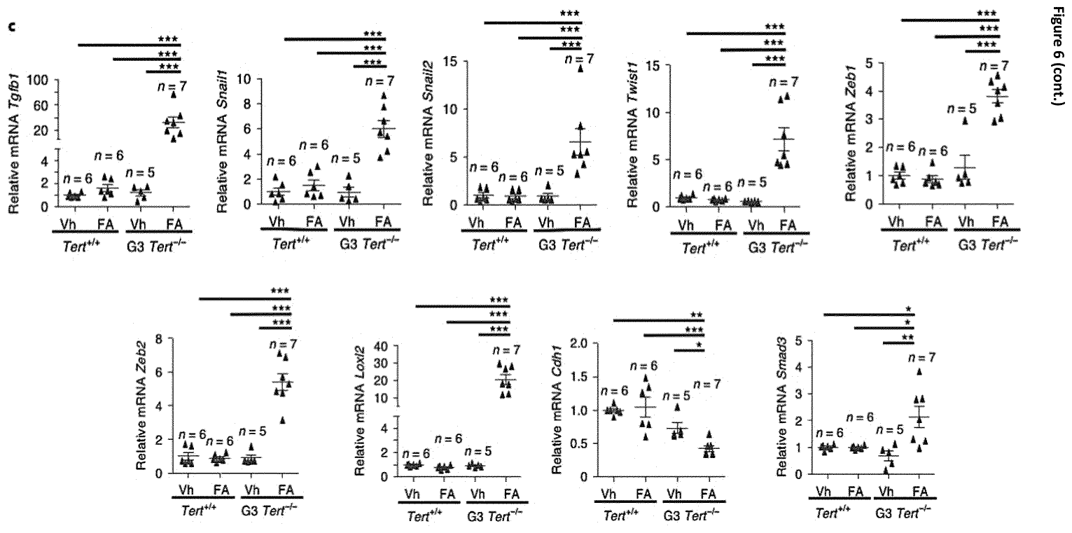

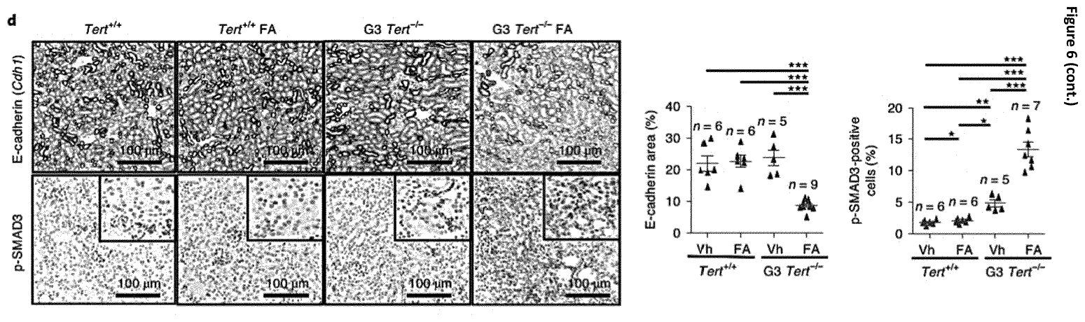

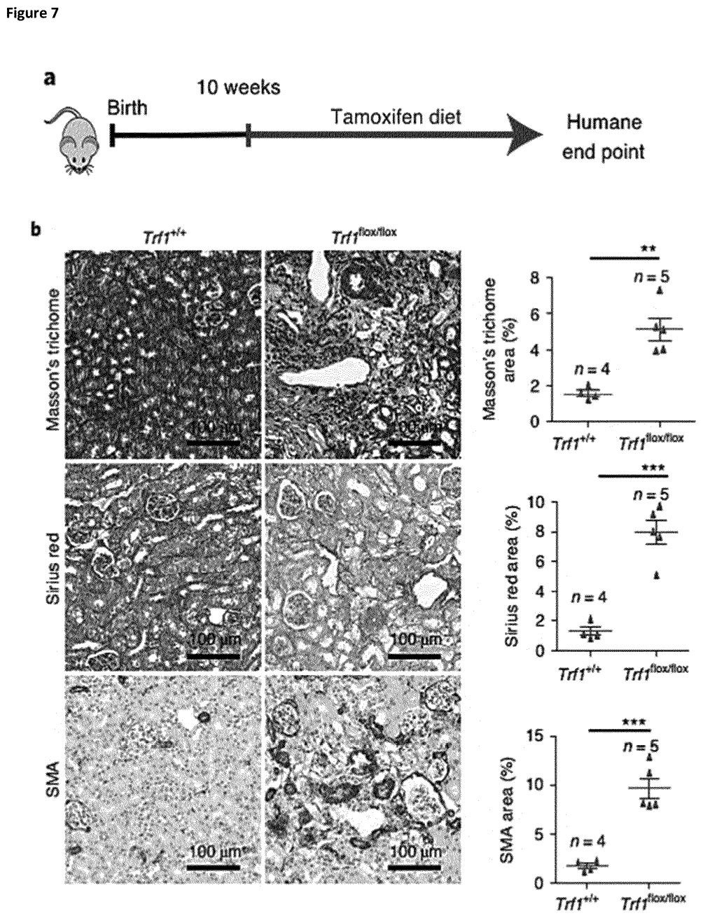

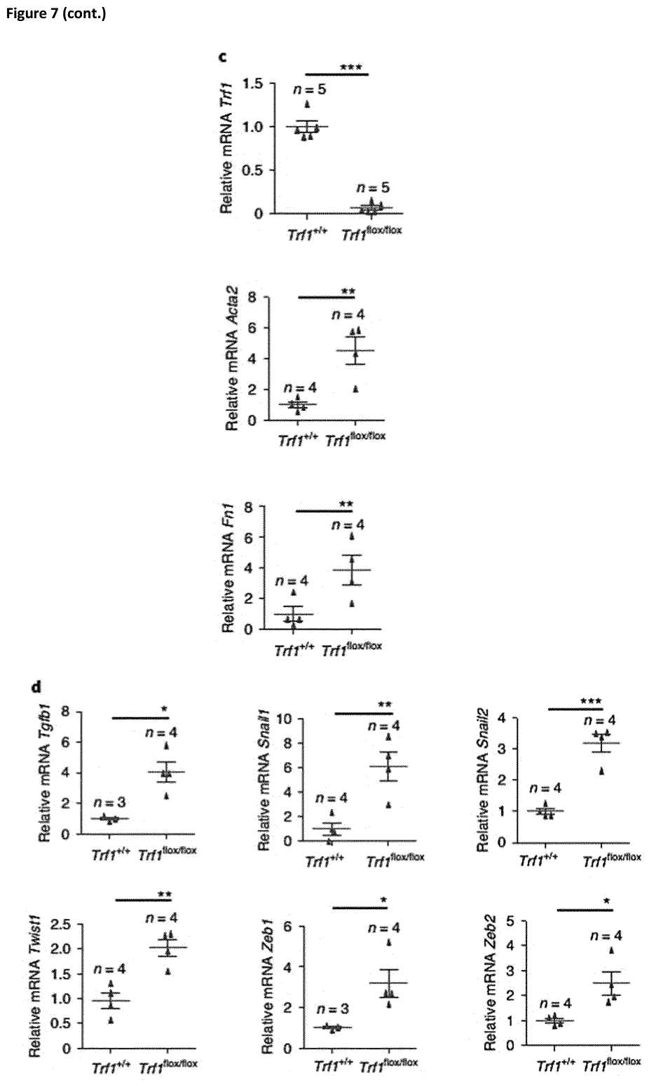

In addition, to assess the contribution of dysfunctional telomeres to the induction of kidney fibrosis, we used a second model of telomere dysfunction by deleting TRF1, one of the components of the shelterin telomere protective complex. In particular, we used a mouse model in which treatment with tamoxifen led to deletion of Trf1 in all kidney cells (FIG. 7). The results showed that mice with dysfunctional telomeres owing to deletion of Trf1 spontaneously develop kidney fibrosis, highlighting the importance of proper telomere function in the protection of fibrotic pathologies.

Overall, the present invention provides suitable mice models suitable to study kidney fibrosis associated to the telomere length. Said mouse models are key tools to understand the role of short and dysfunctional telomeres and resulting DNA damage in the molecular events associated with fibrosis, particularly kidney fibrosis. They will also allow the development of gene therapy approaches aimed at correcting the accumulation of critically short telomeres that is associated with said conditions.

In view of the above, the present invention provides, in a first aspect, compositions and methods useful for the treatment and prevention of a condition associated with short telomere length, particularly kidney fibrosis, to a subject in need thereof. A “condition associated with short telomere length” is one which is characterized by an accumulation of critically short telomeres. In certain embodiments, the condition associated with short telomere length is characterized by mutations in a gene or genes involved in telomere maintenance. Specific examples of such genetically based conditions include, but are not limited to, kidney fibrosis, preferably kidney fibrosis associated with telomere shortening. Short telomeres exacerbate epithelial-to-mesenchymal transition (EMT) program in the kidneys and thus promote pathological scarring of kidney tissue, i.e. fibrosis. Kidney fibrosis associated with telomere shortening is characterised by the presence of short/dysfunctional telomeres owing to mutations in genes related to telomere maintenance, being the most frequently mutated those encoding proteins of the telomerase complex (i.e. TERT, TERC, NOP10, DKC1, NHP2). Both a functional telomerase complex and a proper telomere capping structure by the shelterin proteins are required for maintenance and capping of chromosome ends, respectively.

In some embodiments, the kidney fibrosis is characterised by abnormal production and deposition of extracellular matrix (ECM) proteins mainly in the kidney interstitium and results in structural damage, impairment of renal function, and eventually end-stage renal disease (ESRD). The clinical features of patients suffering from kidney fibrosis are swollen ankles, feet or hands, shortness of breath, tiredness, blood in urine, insomnia, itchy skin, muscle cramps, and headache, among others. Kidney fibrosis is a direct consequence of the kidney's limited capacity to regenerate after injury. In some embodiments, kidney fibrosis is characterized by fibroblast activation and excessive production and deposition of extracellular matrix (ECM), leading to destruction of renal parenchyma, an inflammatory and fibrotic response and decreased renal function. Please note that kidney fibrosis is synonymous of renal fibrosis.

Accordingly, the invention provides compositions and methods for treating a subject in need thereof who is suffering from a condition associated with short telomere length, preferably kidney fibrosis, comprising administering to the patient an agent which increases the telomere length of the patient. The present invention also relates to method of preventing said condition. “Preventing”, “to prevent” or “prevention” or any other similar term, include without limitation, decreasing, reducing or ameliorating the risk of a symptom, disorder, condition, or disease, and protecting an animal from a symptom, disorder, condition, or disease. A prevention may be applied or administered prophylactically. “Treating”, “to treat” or “treatment” or any other similar term, include without limitation, restraining, slowing, stopping, reducing, ameliorating, or reversing the progression or severity of an existing symptom, clinical sign, disorder, condition, or disease. A treatment may be applied or administered therapeutically.

“Subjects in need thereof” refers herein to subjects suffering from a condition that exhibit premature onset of pathologies resulting from a defective regenerative capacity of kidney tissues. In certain embodiments, subjects in need of said treatment exhibit a fibrotic response, preferably kidney fibrotic response, and/or a decreased renal function. In some embodiments, subjects in need of said treatment present a disease or condition that implies or eventually leads to kidney fibrosis, such as glomerulosclerosis, renal interstitial fibrosis, diabetes, hypertension, infectious glomerulonephritis, renal vasculitis, ureteral obstruction, genetic alterations, autoimmune diseases, or any combination thereof. Preferably, the subject in need of said treatment suffers from kidney fibrosis, preferably kidney fibrosis associated with telomere shortening. The terms “patient” or “subject” are considered herein synonymous and refer to a mammal. In certain embodiments the patient is a rodent, primate, human, ungulate, cat, dog, mouse, rat, rabbit, pig, horse, sheep, cow, domestic cat or dog, or other domestic pet or domesticated mammal. In a preferred embodiment, the patient or the subject in need thereof is a human.

### Compositions and Methods

In one embodiment, the agent prevents degradation of the chromosomal ends. In one embodiment, the agent increases the activity of telomerase reverse transcriptase (TERT). In one embodiment, the method of treatment is a gene therapy method that comprises administering to the patient a nucleic acid vector comprising a coding sequence for telomerase reverse transcriptase (TERT).

In certain embodiments, the TERT sequence used in the gene therapy vector is derived from the same species as the subject. For example, gene therapy in humans would be carried out using the human TERT sequence. In one embodiment, the TERT is encoded by the nucleic acid sequence as set forth in SEQ ID NO: 1 or SEQ ID NO: 3 (human TERT variants 1 and 2), or is an active fragment or functional equivalent of SEQ ID NO: 1 or SEQ ID NO: 3. The polypeptide sequence encoded by SEQ ID NO: 1 is set forth in SEQ ID NO: 2. The polypeptide encoded by SEQ ID NO: 3 is set forth in SEQ ID NO: 4. As used herein, “functional equivalent” refers to a nucleic acid molecule that encodes a polypeptide that has TERT activity or a polypeptide that has TERT activity. The functional equivalent may displays 50%, 60%, 70%, 80%, 90%, 95%, 98%, 99%, 100% or more activity compared to TERT encoded by SEQ ID NO: 1 or SEQ ID NO: 3. In an embodiment, the TERT is encoded by a nucleic acid sequence comprising or consisting of the sequence of SEQ ID NO: 1 or SEQ ID NO: 3. Functional equivalents may be artificial or naturally-occurring. For example, naturally-occurring variants of the TERT sequence in a population fall within the scope of functional equivalent. TERT sequences derived from other species also fall within the scope of the term “functional equivalent”, in particular the murine TERT sequence given in SEQ ID NO: 5. In a particular embodiment, the functional equivalent is a nucleic acid with a nucleotide sequence having at least 75%, 80%, 85%, 90%, 95%, 96%, 97%, 98%, 99%, 99.5%, 99.9% identity to SEQ ID NO: 1 or SEQ ID NO: 3. In a further embodiment, the functional equivalent is a polypeptide with an amino acid sequence having at least 75%, 80%, 85%, 90%, 95%, 96%, 97%, 98%, 99%, 99.5%, 99.9% identity to SEQ ID NO: 2 or SEQ ID NO: 4. In an embodiment, the TERT comprises or consists of an amino acid sequence of SEQ ID NO: 2 or SEQ ID NO: 4. In the case of functional equivalents, sequence identity should be calculated along the entire length of the nucleic acid. Functional equivalents may contain one or more, e.g. 2, 3, 4, 5, 10, 15, 20, 30 or more, nucleotide insertions, deletions and/or substitutions when compared to SEQ ID NO: 1 or SEQ ID NO: 3. The term “functional equivalent” also encompasses nucleic acid sequences that encode a TERT polypeptide with at least 75%, 80%, 85%, 90%, 95%, 96%, 97%, 98%, 99%, 99.5%, 99.9% sequence identity to the sequence as set forth in SEQ ID NO: 2 or SEQ ID NO: 4, but that show little homology to the nucleic acid sequence given in SEQ ID NO: 1 or SEQ ID NO: 3 because of the degeneracy of the genetic code. Sequence identity may be calculated by any one of the various methods in the art, including for example BLAST and variations on these alignment programs.

As used herein, the term “active fragment” refers to a nucleic acid molecule that encodes a polypeptide that has TERT activity or polypeptide that has TERT activity, but which is a fragment of the nucleic acid as set forth in SEQ ID NO: 1 or SEQ ID NO: 3 or the amino acid sequence as set forth in SEQ ID NO: 2 or SEQ ID NO: 4. An active fragment may be of any size provided that TERT activity is retained. A fragment will have at least 75%, 80%, 85%, 90%, 95%, 96%, 97%, 98%, 99%, 99.5%, 100% identity to SEQ ID NO: 1-4 along the length of the alignment between the shorter fragment and SEQ ID NO: 1-4.

Fusion proteins including these fragments can be comprised in the nucleic acid vectors needed to carry out the invention. For example, an additional 5, 10, 20, 30, 40, 50 or even 100 amino acid residues from the polypeptide sequence, or from a homologous sequence, may be included at either or both the C terminal and/or N terminus without prejudicing the ability of the polypeptide fragment to fold correctly and exhibit biological activity.

In one embodiment, the method of treatment is a gene therapy method and/or the nucleic acid vector used is a gene therapy vector. Gene therapy methods and vectors are well known in the art and generally comprise delivering a nucleic acid encoding a therapeutically active protein to a subject. The nucleic acid may be delivered in a number of ways and forms including delivering naked deoxyribonucleic acid (DNA) such as plasmid or mini-circles, delivering ribonucleic acids (RNA), such as messenger RNA, the use of liposomes or cationic polymers or other engineered nano-particles containing the nucleic acid, or viral vectors that encapsidate the nucleic acid.

In a further embodiment, the gene therapy is achieved using stable transformation of organisms with an inducible expression system. Suitable inducible expression systems are known in the art and include the CRE-LOX recombinase based system which is suitable for use in mice and tetracycline-regulated which can be used in the treatment of human subjects.

In one embodiment the gene therapy vector is a ribonucleic acid (RNA) vector comprising or consisting of the coding sequence for telomerase reverse transcriptase (TERT), as defined above. In some embodiments, the RNA nucleic acid vector is delivered in a naked form, i.e., unshielded RNA. In some other embodiments, the RNA nucleic acid vector is modified as to be protected from nuclease degradation. Said modifications include chemical modifications in the structure of the RNA, such as those performed in the 2′ position, in the phosphate linkage and/or in the nucleobase. Other modifications include the encapsidation of the RNA nucleic acid vector into nanoparticles, polymers, endosomes, lipids and lipid-like particles, or other delivery vectors, such as viruses. Nanoparticle encapsulation of RNA physically protects nucleic acids from degradation and, depending on the specific chemistry, can aid in cellular uptake and endosomal escape. Polymers include Poly(β-amino esters), poly-L-lysine, polyamidoamine, and polyethyleneimine, as well as naturally occurring polymers such as chitosan, have all been applied to RNA delivery. Chemically well-defined means of delivery is to directly conjugate a bioactive ligand, such as N-acetylgalactosamine, cholesterol, vitamin E, antibodies, peptides, to the RNA that will allow it to enter the cell of interest.

Preferably, the RNA nucleic acid vector is a single stranded RNA molecule, more preferably a messenger RNA (mRNA) vector. The delivery of therapeutics mRNA vectors has been facilitated by maximizing the translation and stability of the mRNA vector, preventing its immune-stimulatory activity and the development of in vivo delivery technologies. In some embodiments, the mRNA vector is modified to increase the in vivo delivery efficacy. In an embodiment, the mRNA vector is modified to include the incorporation of a 5′ cap and/or a 3′ poly(A) tail to efficient translation and prolonged half-life of mature mRNAs. Cap analogues such as ARCA (anti-reverse cap analogues) and poly(A) tails of 120-150 bp can also be used. New types of cap analogues, such as 1,2-dithiodiphosphate-modified caps, with resistance against RNA decapping complex, are also included. The so-called codon optimization also facilitates better efficacy of protein synthesis and limits mRNA destabilization by rare codons. Similarly, engineering 3′ and 5′ untranslated regions (UTRs), which contain sequences responsible for recruiting RNA-binding proteins (RBPs) can enhance the level of protein product. N1-methyl-pseudouridine base modification is usually used to mask mRNA immune-stimulatory activity.

In one embodiment, the regulatory sequence operatively linked to the TERT coding sequence is the cytomegalovirus promoter (CMV), although other suitable regulatory sequences will be known to those of skill in the art. In other embodiments, the regulatory sequence operatively linked to the TERT coding sequence is a kidney-specific promoter. In a preferred embodiment, the coding sequence for telomerase reverse transcriptase (TERT) gene comprised in the RNA nucleic acid vector is operably linked to a CMV or a kidney-specific promoter, wherein the RNA nucleic acid vector further comprises a poly(A) sequence placed at the end of the coding sequence for the telomerase reverse transcriptase (TERT) gene.

In one embodiment the gene therapy vector is a viral vector. Viral gene therapy vectors are well known in the art. Vectors include integrative and non-integrative vectors such as those based on retroviruses, adenoviruses (AdV), adeno-associated viruses (AAV), Antiviruses, pox viruses, alphaviruses, and herpes viruses. Using non-integrative viral vectors, such as AAV, seems to be particularly advantageous. In one aspect, this is because non-integrative vectors do not cause any permanent genetic modification. Second, the vectors target to adult tissues, avoiding having the subjects under the effect of constitutive telomerase expression from early stages of development. Additionally, non-integrative vectors effectively incorporate a safety mechanism to avoid over-proliferation of TERT expressing cells. Cells will lose the vector (and, as a consequence, the telomerase expression) if they start proliferating quickly.

Particular examples of suitable non-integrative vectors include those based on adenoviruses (AdV) in particular gutless adenoviruses, adeno-associated viruses (AAV), integrase deficient lentiviruses, poxviruses, alphaviruses, and herpesviruses. Preferably, the non-integrative vector used in the invention is an adeno-associated virus-based non-integrative vector, similar to natural adeno-associated virus particles.

Vectors derived from adeno-associated viruses (AAVs) have emerged as one of the vectors of choice for many gene transfer applications because of their many desirable properties, including capability to transduce a broad range of tissues at high efficiency, poor immunogenicity and an excellent safety profile, toxicity being absent in many preclinical models. AAV vectors transduce post-mitotic cells and can sustain long-term gene expression (up to several years) both in small and large animal models of disease. Safety and efficacy of AAV gene transfer has been extensively studied in humans with encouraging results in the liver, muscle, CNS, and retina.

AAV preferentially target post-mitotic tissues, which are considered more resistant to cancer than the highly proliferative ones. Examples of adeno-associated virus-based non-integrative vectors include vectors based on any AAV serotype, i.e. AAV1, AAV2, AAV3, AAV4, AAV5, AAV6, AAV7, AAV8, AAV9, AAV10, AAV11 and pseudotyped AAV. Tissue specificity is determined by the capsid serotype. Pseudotyping of AAV vectors and capsid engineering to alter their tropism range will likely be important to their use in therapy.

AAV2 is the best characterized serotype for gene transfer studies both in humans and experimental models. AAV2 presents natural tropism towards skeletal muscles, neurons, vascular smooth muscle cells and hepatocytes. AAV2 is therefore a good choice of vector to target these tissues, in particular when using the methods or vectors of the invention to treat a condition associated with one of these tissues. For example, treatment of neuromuscular degeneration may be targeted to skeletal muscle and/or neurons in this way.

Newly isolated serotypes, such as AAV7, AAV8, and AAV9 have been successfully adopted in preclinical studies, long term expression of the therapeutic gene is possible depending on the target tissue and the route of administration. In addition, the use of non-human serotypes, like AAV8 and AAV9, might be useful to overcome these immunological responses in subjects, and clinical trials have just commenced (ClinicalTrials.gov Identifier: NCT00979238).

Altogether, these encouraging data suggest that AAV vectors are useful tools to treat human diseases with a high safety and efficient profile.

The choice of adeno-associated viruses of wide tropism, such as those derived from serotype 9 adeno-associated virus (AAV9) is particularly advantageous when treating conditions associated with short telomere length. AAV9 viruses have shown efficient transduction in a broad range of tissues, with high tropism for liver, heart and skeletal muscle and thus the beneficial effects of gene therapy can be achieved in more tissues. In addition, AAV9 vectors have the unique ability to cross the blood-brain-barrier and target the brain upon intravenous injection in adult mice and cats (Foust et al Nature biotechnology 2009).

One aspect of the invention provides a system in which the capsid (which is the part of the virus which determines the virus tropism) of the adeno-associated virus-based vector is made of capsid proteins of the serotype 9 adeno-associated virus (AAV9). In one embodiment of the viral vectors for use in the invention, the polynucleotide sequence packed in the capsid is flanked by internal terminal repeats (ITRs) of an adeno-associated virus, preferably of serotype 2 which has been extensively characterised in the art and presents a coding sequence located between the ITRs. As set out above, the nucleic acid preferably codes for a functional TERT polypeptide. In one embodiment, the regulatory sequence operatively linked to the TERT coding sequence is the cytomegalovirus promoter (CMV), although other suitable regulatory sequences will be known to those of skill in the art. In another embodiment, the regulatory sequence operatively linked to the TERT coding sequence is a kidney-specific promoter.

When treating conditions associated with short telomere length, it is advantageous to target the treatment to the effected tissues. The choice of AAV serotype for the capsid protein of the gene therapy vector may be thus based on the desired site of gene therapy. If the target tissue is skeletal muscle, for example, in treating loss of neuromuscular coordination, AAV1- and AAV6-based viral vectors can be used. Both of these serotypes are more efficient at transfecting muscle than other AAV serotypes.

Alternatively, other viral vectors can be used in the present invention. Any vector compatible with use in gene therapy can be used in the present invention. Heilbronn & Weger (2010) Handb Exp Pharmacol. 197: 143-70 provides a review of viral vectors that are useful in gene therapy.

In accordance with all the previous discussion, vectors comprising a coding sequence for telomerase reverse transcriptase (TERT) suitable for use in gene therapy are an important point for putting the invention into practice. Suitable gene therapy vectors include any kind of particle that comprises a polynucleotide fragment encoding the telomerase reverse transcriptase (TERT) protein, operably linked to a regulatory element such as a promoter, which allows the expression of a functional TERT protein demonstrating telomerase reverse transcriptase activity in the targeted cells. Preferably, TERT is encoded by the nucleic acid sequence as set forth in SEQ ID NO: 1 or SEQ ID NO: 3, or is an active fragment or functional equivalent of TERT. The term gene therapy vector includes within its scope naked DNA molecules such as plasmids or mini-circles, i.e. circular DNA molecules which do not contain bacterial DNA sequences, provided that the TERT coding sequence and its linked regulatory element are inserted in the plasmid, as well as to more complicated systems, such as particles with the structure of virions (viral particles), comprising at least a capsid and at least a polynucleotide sequence, with a size that allows the polynucleotide sequence to be packed within the capsid in a manner similar to that of the native genome of the virus of origin of the capsid. The polynucleotide sequence must include a region where the TERT coding sequence and its linked regulatory element are inserted such that the telomerase reverse transcriptase protein can be expressed from that polynucleotide sequence once the viral particle has infected a cell.

In one embodiment, the gene therapy vector suitable for being used in the invention is a non-integrative vector, such as an adeno-associated virus-based non-integrative vector. For the purposes of the invention, the choice of non-integrative vectors seems to be particularly advantageous, because they do not cause any permanent genetic modification. Also, as stated before, such vectors incorporate a safety mechanism to avoid over-proliferation of TERT expressing cells that will lose the vector if the cells start proliferating quickly.

Adeno-associated virus-based vectors derived from a serotype 9 adeno-associated virus (AAV9) are preferred because the beneficial effects can be achieved in more tissues (see above). In one particularly preferred embodiment, the regulatory sequence operatively linked to the TERT coding sequence is the cytomegalovirus promoter (CMV). The nucleic acid sequence encoding TERT is operably linked to a regulatory sequence that drives the expression of the coding sequence. As used herein, the term “regulatory element” means a nucleic acid sequence that serves as a promoter, i.e., regulates expression of a nucleic acid sequence operably linked to the promoter. Such “regulatory elements” or “promoters” can control the expression of linked nucleic acid sequences either constitutively or inducible. The regulatory sequence may be a constitutive promoter. An example of a regulatory sequence which is a constitutive promoter is the cytomegalovirus (CMV) promoter.

The expression of TERT following gene therapy according to the invention persists for a time of several months to several years. In one embodiment of the invention, the subject is treated once. In an alternative embodiment, the subject is treated initially, and is then treated again once TERT expression levels decrease by about 50% of those attained immediately following treatment. Treatment may be repeated with the same or alternative vector to maintain the reduction in age-related disorders if necessary, for example annually, or once every 5 years or once a decade. When administering a second or subsequent dose, it may be necessary to use a different gene therapy vector, for example when using an AAV-based vector the second and subsequent administrations may be a vector with a capsid derived from a different serotype than that used for the first administration.

The methods of treatment of the invention have the effect of treating and/or preventing conditions associated with short telomere length. In a further aspect, therefore, the invention refers to a gene therapy method or the use of a nucleic acid vector as described above, for use in the treatment or prevention of kidney fibrosis, preferably kidney or fibrosis associated with short telomere length, in a subject in need thereof.

The effectiveness of treatment of the conditions associated with short telomere length can be measured by various methods known in the art. In one embodiment, the effectiveness of the treatment is measured by an increase in lifespan of a treated patient suffering from a condition associated with short telomere length as compared to the expected lifespan of an untreated patient suffering from the same condition. In certain embodiments, the lifespan is extended by 5%, 10%, 15%, 20% or more, with reference to the expected lifespan for a patient suffering from the same condition. In one embodiment, the effectiveness of the treatment is measured by a delayed or prevented kidney failure in a treated patient suffering from a condition associated with short telomere as compared to the expected onset kidney failure in an untreated patient suffering from the same condition. In certain embodiments, the delay in the onset of bone marrow failure of a treated patient suffering from a condition associated with short telomere length is extended by 5%, 10%, 15%, 20% or more, with reference to the expected onset of kidney failure for an untreated patient suffering from the same condition.

In one embodiment, the effectiveness of the treatment is measured by an increase in overall fitness of a treated patient suffering from a condition associated with short telomere length treated, preferably kidney fibrosis, as compared to the overall fitness of an untreated patient suffering from the same condition. Overall fitness can be determined by measuring physical attributes associated with the particular condition. Thus, an increase in overall fitness can be determined by a decrease in physical attributes associated with the particular condition exhibited by the treated patient. In one embodiment, increased overall fitness is measured by determining the estimated glomerular filtration rate (eGFR) and/or albuminuria (ACR). A glomerular filtration rate (GFR) of 60 or higher is in the normal range. A GFR below 60 may mean kidney disease. A GFR of 15 or lower may mean kidney failure. The standardized albumin-to-creatinine ratio (ACR) test shows whether you have albumin in your urine. A normal amount of albumin in your urine is less than 30 mg/g. Anything above 30 mg/g may mean you have kidney disease, even if your GFR number is above 60. In certain embodiments, the glomerular filtration rate in a treated patient is increased by 5%, 10%, 15%, 20% or more, with reference to the glomerular filtration rate of an untreated patient suffering from the same condition.

The efficacy of the treatment can also be measured by directly determining telomere length in sample taken from the patient. Telomere length can be measured, for example, by using standard hybridization techniques, such as fluorescence in situ hybridization (FISH), Quantitative Fluorescent in situ hybridization (Q-FISH), or High Throughput Quantitative Fluorescent in situ hybridization (HT Q-FISH) in a sample taken from the patient. Samples suitable for telomere analysis include blood, urine, or tissue biopsies, such as kidney biopsies.

In a particular embodiment, samples are taken from the patient undergoing treatment throughout the course of the treatment so that both absolute telomere length and the rate of telomere shortening over the course of treatment can be determined. Samples may be taken every day during the course of treatment, or at longer intervals. In one embodiment, samples are taken once a week, once every two week, once every three weeks, once every 4 weeks, once every five weeks, once every six weeks or longer. Comparison of telomere length can be measured by a comparing the proportion of short telomeres in a sample taken from a patient. In one embodiment, the proportion of short telomeres is the fraction of telomeres presenting an intensity below the mean intensity of the sample as measured by a in situ hybridization technique, such as FISH or Q-FISH. In embodiment, the proportion of short telomeres is the fraction of telomeres presenting an intensity 75%, 70%, 65%, 60%, 55%, 50%, 45%, 40% or more below the mean intensity of the sample. In one particular embodiment, the proportion of short telomeres is the fraction of telomeres presenting an intensity 50% or more below the mean intensity of the sample.

In another embodiments, the proportion of short telomeres is the fraction of telomeres below a certain length, e.g. 8 kb, 7 kb, 6 kb, 5 kb, or shorter. In one embodiment, the proportion of short telomeres is the fraction of telomeres 8 kb or shorter. In another embodiment, the proportion of short telomeres is the fraction of telomeres 7 kb or shorter. In another embodiment, the proportion of short telomeres is the fraction of telomeres 6 kb or shorter. In another embodiment, the proportion of short telomeres is the fraction of telomeres 5 kb or shorter. In another embodiment, the proportion of short telomeres is the fraction of telomeres 4 kb or shorter. In another embodiment, the proportion of short telomeres is the fraction of telomeres 3 kb or shorter.

In one embodiment, the effectiveness of the treatment is measured by a decrease in the proportion of short telomeres in sample taken from a treated patient suffering from a condition associated with short telomere length, preferably kidney fibrosis, as compared to a control sample. In one embodiment, the proportion of short telomeres in a sample taken from a treated patient is decreased by 10%, 20%, 30%, 40%, 50%, 60%, 70%, or greater as compared to a control sample. In one embodiment, the control sample is a sample taken from the same patient prior to the treatment or taken at an earlier stage of the treatment. In another embodiment, the control sample is a sample taken from a patient suffering from the same condition and not provided the treatment.

In a further aspect, the invention is applied to the subject by administering a pharmaceutical composition comprising an effective amount of any one of the gene therapy vectors compatible with the invention described above.

A “pharmaceutical composition” is intended to include the combination of an active agent with a carrier, inert or active, making the composition suitable for diagnostic or therapeutic use in vitro, in vivo or ex vivo.

“Composition” is intended to mean a combination of active agent and another compound or composition, inert (for example, a detectable agent or label) or active. An “effective amount” is an amount sufficient to effect beneficial or desired results. An effective amount can be administered in one or more administrations, applications or dosages.

They will usually include components in addition to the active component (such as the gene therapy vector) e.g. they typically include one or more pharmaceutical carrier(s) and/or excipient(s).

Compositions will generally be administered to a subject in aqueous form. Prior to administration, however, the composition may have been in a non-aqueous form. For instance, although some viral vectors are manufactured in aqueous form, then filled and distributed and administered also in aqueous form, other viral vectors are lyophilised during manufacture and are reconstituted into an aqueous form at the time of use. Thus, a composition of the invention may be dried, such as a lyophilised formulation. The composition may include preservatives such as thiomersal or 2-phenoxyethanol. It is preferred, however, that the composition should be substantially free from (i.e. less than 5 μg/m′i′) mercurial material e.g. thiomersal-free.

To control tonicity, it is preferred to include a physiological salt, such as a sodium salt. Sodium chloride (NaCl) is preferred, which may be present at between 1 and 20 mg/ml e.g. about 10+2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The composition may include material for a single administration or may include material for multiple administrations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Compositions of the invention for use in humans are typically administered in a dosage volume of about 0.5 ml, although a half dose (i.e. about 0.25 ml) may be administered to children.

As well as methods of treatment described herein, the invention also provides a nucleic acid sequence encoding a TERT for use in therapy. The invention also provides a nucleic acid vector comprising a coding sequence for telomerase reverse transcriptase (TERT), for use in a method of therapy and a gene therapy vector comprising a coding sequence for telomerase reverse transcriptase (TERT), for use in a method of therapy. In particular, the therapy may be treating or preventing a condition associated with short telomere length. As described for methods of treatment, the TERT nucleic acid sequence may be the sequence as recited in SEQ ID NO: 1 or SEQ ID NO: 3 or a fragment or functional equivalent thereof. The TERT protein may have a sequence as recited in SEQ ID NO: 2 or SEQ ID NO: 4, or a fragment or functional equivalent thereof.

The following embodiments are also derived from the first aspect and included herein:

1. A method of treating a patient with kidney fibrosis associated with short telomere length comprising administering to the patient a nucleic acid vector comprising a coding sequence for telomerase reverse transcriptase (TERT).

2. The method of 1, wherein TERT is encoded by a nucleic acid sequence comprising a sequence that is at least 90% identical to the sequence of SEQ ID NO: 1 or SEQ ID NO: 3.

3. The method of 1 or 2, wherein TERT is encoded by a nucleic acid sequence comprising the sequence of SEQ ID NO: 1 or SEQ ID NO: 3.

4. The method of any of 1-3, wherein TERT is encoded by a nucleic acid sequence consisting of the sequence of SEQ ID NO: 1 or SEQ ID NO: 3

5. The method of any of 1-4, wherein TERT comprises an amino acid sequence that is at least 90% identical to the amino acid sequence of SEQ ID NO:2 or SEQ ID NO: 4.

6. The method of any of 1-5, wherein TERT comprises an amino acid sequence of SEQ ID NO:2 or SEQ ID NO: 4.

7. The method of any of 1-6, wherein TERT consists of the amino acid sequence of SEQ ID NO:2 or SEQ ID NO: 4.

8. The method of any of 1-7, wherein the nucleic acid sequence encoding TERT is operably linked to a regulatory sequence that drives the expression of the coding sequence.

9. The method of any of 1-8, wherein the vector is a non-integrative vector.

10. The method of any of 1-9, wherein the vector is an adeno-associated virus-based non-integrative vector or a RNA vector.

11. The method of any of 1-10, wherein the vector is an adeno-associated virus-based vector derived from a serotype 9 adeno-associated virus (AAV9).

12. The method of 11, wherein the capsid of the adeno-associated virus-based vector is made of capsid proteins of the serotype 9 adeno-associated virus (AAV9), and the nucleic acid sequence contained in the capsid is flanked at both ends by internal terminal repeats corresponding to serotype 2 adenoassociated viruses.

13. The method of 12, wherein the nucleic acid contained in the capsid comprises a fragment which encodes the amino acid sequence coding for TERT.

14. The method of any of 1-13, wherein the vector comprises a regulatory sequence which is a constitutive promoter.

15. The method of 14, wherein the regulatory sequence is the cytomegalovirus (CMV) promoter.

16. The method of any of 1-15, wherein the kidney fibrosis associated with short telomere length is characterized by mutations in a gene or genes involved in telomere maintenance.

### Non-Human Knock-Out Animal Model

The invention further provides in a second aspect a non-human knock-out animal model characterized in that it exhibits a pathological condition of kidney fibrosis, wherein said non-human animal is obtained or obtainable when a sublethal dose of folic acid is administered to a non-human knock-out animal comprising a deactivation of at least one, preferably both, alleles of the Tert gene. By “deactivation of at least one, preferably both, alleles of the Tert gene” is referred herein to the process in which the sequence of one or both alleles of the Tert gene is altered thereby causing said allele(s) to lose its biological function. The alteration in the sequence of the allele(s) may be by inserting nucleotides into said allele(s) or by partially or totally deleting the nucleotides of said allele(s).

Specifically, the non-human knock-out animal model has a knock-out mutation in the Tert gene, resulting in the knock-out of Tert. The term “knock-out animal” as used herein refers to a non-human animal, preferably a mammal, which carries one or more genetic manipulations leading to deactivation of the Tert gene, either only one allele or preferably both alleles of said gene. Said knock-out mutation of one or both of the alleles of the Tert gene may be present in the animal's germ cells, somatic cells, or in both (i.e., the knock-out mutation of the Tert gene is in all the animal's cells). By “germ cells” is referred herein as the germ cells or gametes (eggs and sperm). By “somatic cells” is referred herein as the cells forming the body of a multicellular organism other than germ cells or gametes.

In a preferred embodiment, said non-human knock-out animal is a rodent, such as a mouse or rat. The male animal founders are referred as G0, while the first, second, third generation are referred to as G1, G2, G3, respectively. Preferably, the non-human knock-out animal is the third generation (G3) or subsequent generations (G4, G5, etc) of the Tert^{-/-} know-out animal lineage. Preferably, the non-human animal model is a G3 Tert^{-/-} mouse. The expression “G3 Tert^{-/-}” refers herein to the third generation of animals, preferably mice, that comprise a deactivation in both alleles of the Tert gene, preferably wherein said deactivation is hereditary, i.e., it is present in the germ cells of the animal. To produce a colony of Tert^{-/-} animals, heterozygous animals Tert^{+/+} are intercrossed to produce homozygous animals Tert^{-/-} for further phenotyping. The knock-out technology is well known in the art.

Embryonal cells at various developmental stages can be used to introduce genes for the production of knock-out animals. Different methods are used depending on the stage of development of the embryonal cell. Such transfected embryonic stem (ES) cells can thereafter colonize an embryo following their introduction into the blastocoele of a blastocyst-stage embryo and contribute to the germ cells of the resulting chimeric animal. Prior to the introduction of transfected ES cells into the blastocoele, the transfected ES cells can be subjected to various selection protocols to enrich the proportion of ES cells that have integrated into knock-out gene if the knock-out gene provides a means for such selection. Alternatively, PCR can be used to screen for ES cells that have integrated the knock-out.

In addition, retroviral infection can also be used to introduce knock-out genes into a non-human animal. The developing non-human embryo can be cultured in vitro to the blastocyst stage. During this time, the blastomeres can be targets for retroviral infection. Efficient infection of the blastomeres is obtained by enzymatic treatment to remove the zona pellucida. The viral vector system used to introduce the knock-out is typically a replication-defective retrovirus carrying the knock-out. Transfection is easily and efficiently obtained by culturing the blastomeres on a monolayer of virus-producing cells. Alternatively, infection can be performed at a later stage. Virus or virus-producing cells can be injected into the blastocoele. Most of the founders will be mosaic for the knock-out gene since incorporation occurs only in a subset of cells, which form the knock-out animal. Further, the founder can contain various retroviral insertions of the knock-out gene at different positions in the genome, which generally will segregate in the offspring. In addition, knock-out genes may be introduced into the germline by intrauterine retroviral infection of the mid-gestation embryo. Additional means of using retroviruses or retroviral vectors to create knock-out animals known to those of skill in the art involve the micro-injection of retroviral particles or mitomycin C-treated cells producing retrovirus into the perivitelline space of fertilized eggs or early embryos.

Once the founder animals are produced, they can be bred, inbred, outbred, or crossbred to produce colonies of the particular animal. The knock-out animals are screened and evaluated to select those animals having the phenotype of interest. Initial screening can be performed using, for example, Southern blot analysis or PCR techniques to analyze animal tissues to verify that the knock-out gene has taken place. The know-out animals can be identified by assessing the levels of TERT mRNA expression in the tissues of the knock-out animals using techniques which include, but are not limited to, Northern blot analysis of tissue samples obtained from the animal, in situ hybridization analysis, and reverse transcriptase-PCR (rt-PCR). Samples of the suitable tissues can be evaluated immunocytochemically using antibodies specific for Tert protein or with a tag such as EGFP. The knock-out non-human mammals can be further characterized to identify those animals having a phenotype useful in the invention. In particular, knock-out non-human mammals having one or both Tert alleles deactivated can be screened according to the presence of kidney pathology or fibrosis once a sublethal dose of FA has been administered to them.

As shown in the examples below, the a G3 Tert^{-/-} non-human knock-out mouse only becomes a non-human animal model for kidney fibrosis when a sublethal dose of FA has been administered to it. Therefore, it is noted that the non-human animal model according to the second aspect that is characterized in that it is a knock-out for at least one, preferably both of the alleles of the gene coding of the telomerase catalytic subunit Tert, is further characterized for exhibiting a pathological condition of kidney fibrosis upon treatment with a sublethal dose of folic acid. By “sublethal dose” is referred herein as a dose that does not induce kidney fibrosis in wild-type mice. In an embodiment, the sublethal dose is less than 250 mg/kg body weight. Preferably, the dose of FA administered to the non-human animal ranges between 50 and 200 mg/kg, more preferably 50-175 mg/kg, 50-150 mg/kg, 100-150 mg/kg, most preferably of about 125 mg/kg body weight. Preferably, the dose of FA is a dose of up to 200 mg/kg body weight, more preferably of up to 125 mg/kg body weight.

The non-human knock-out animal model for kidney fibrosis is further characterized by having an increased creatinine and BUN levels in the blood upon administration of a sublethal dose of FA. Further, said non-human knock-out animal model presents kidney fibrosis as detected by Masson's trichrome staining and increased renal tubular injury as determined by increased PAS+D staining, as shown in the examples below.

In an embodiment, the dose of FA is administered once, preferably at the start of the study. In another embodiments, the FA is administered several times. In some other embodiments, the FA is administered once per day during several days (such as, once every day, once every two days, once every week, once every two weeks, or once every month). In some embodiments, the dose of FA is administered systemically, preferably intraperitoneally or intravenous.

The non-human knock-out animal to which FA is administered to generate the non-human animal model of the second aspect is between 4-10 weeks of age, preferably between 6-8 weeks of age.

In a third aspect, the invention provides a non-human knock-out animal model for use in studying and/or evaluating evaluate kidney fibrosis associated to the presence of short telomeres, wherein the non-human knock-out animal is as defined in the second aspect or any of its embodiments.

In a fourth aspect, the invention provides methods for producing a non-human knockout animal model for evaluating kidney fibrosis associated to the presence of short telomeres, the method comprising the steps of:
i) providing non-human animals, preferably rodents, characterized by being know-out for one, preferably both alleles of the gene coding the telomerase catalytic subunit Tert, preferably from third generation (G3 Tert⁻/− non-human animal), as defined in the second aspect or any of its embodiments, and
ii) administrating to the non-human animal of i) at least one sublethal dose of folic acid, as also defined in the second aspect or any of its embodiments.

In a fifth aspect, the invention provides a non-human knock-out animal model for use in evaluating kidney fibrosis associated to the presence of short telomeres, wherein said non-human animal is obtained according to the method of the fourth aspect or any of its embodiments.

In a sixth aspect, the invention provides a method of evaluating kidney fibrosis associated to the presence of short telomeres, wherein the method comprises the use of the non-human animal model as defined in the second aspect or any of its embodiments.

In a seventh aspect, invention provides the non-human animal model as defined in the second aspect or any of its embodiments for use in a method for screening compounds to improve, treat or prevent, kidney fibrosis associated to the presence of short telomeres. In a preferred embodiment, the method comprises the steps of:
i) administering a compound to the non-human animal model of kidney fibrosis associated to the presence of short telomeres as defined in the second aspect or any of its embodiments,
ii) detecting or evaluating the symptoms of kidney fibrosis, and
iii) comparing the symptoms of kidney fibrosis with the group of animals not administrated with the compound and selecting the compounds that improved said symptoms, and/or
iv) comparing the symptoms of kidney fibrosis before and after administration of the test compound.

In an eighth aspect, the invention provides a screening method for a test compound for kidney fibrosis associated to the presence of short telomeres, wherein the test compound is administered to the non-human knock-out animal model as defined in the second aspect or any of its embodiments, wherein any change in life expectancy or in symptoms related to kidney fibrosis associated to the presence of short telomeres are measured and evaluated, and wherein the test compound in which said changes were improved is determined as having a therapeutic effect. Particular embodiments of the screening method of the seventh or eighth aspects include steps wherein the tissues, organs or cells derived from said non-human knock-out animal model are contacted with the test compound, and a change in said tissues, organs or cells is measured and evaluated to determine which test compound caused an improvement and therefore has a therapeutic effect.

The test compounds found to have a therapeutic effect for kidney fibrosis associated to the presence of short telomeres which can be obtained by the screening method disclosed herein in the seventh or eighth aspects, can be used for therapy in patients who have developed or are at risk of developing kidney fibrosis associated to the presence of short telomeres. In an embodiment of the screening methods, the case of the non-human animal model according to the second aspect or any of its embodiment is compared and evaluated with the case of a wild-type mouse or with a non-human animal according to the second aspect not treated with the test compound.

In a ninth aspect, the present invention relates to the use of a non-human animal model as defined in the second aspect or any of its embodiments in a screening method for a test compound for kidney fibrosis associated to the presence of short telomeres. In a preferred embodiment, the method comprises the steps of:
i) administering a compound to the non-human animal model of kidney fibrosis associated to the presence of short telomeres as defined in the second aspect or any of its embodiments,
ii) detecting or evaluating the symptoms of kidney fibrosis, and
iii) comparing the symptoms of kidney fibrosis with the group of animals not administrated with the compound and selecting the compounds that improved said symptoms, and/or
iv) comparing the symptoms of kidney fibrosis before and after administration of the test compound and selecting the compounds that improved said symptoms.

It is noted that the embodiments and definitions included under the second aspect of the present invention also apply to the third, fourth, five, sixth, seventh, eighth and ninth aspects.

### SEQUENCE LISTING

SEQ ID NO: 1
caggcagcgc tgcgtcctgc tgcgcacgtg ggaagccctg gccccggcca cccccgcgat
gccgcgcgct ccccgctgcc gagccgtgcg ctccctgctg cgcagccact accgcgaggt
gctgccgctg gccacgttcg tgcggcgcct ggggccccag ggctggcggc tggtgcagcg
cggggacccg gcggctttcc gcgcgctggt ggcccagtgc ctggtgtgcg tgccctggga
cgcacggccg ccccccgccg ccccctcctt ccgccaggtg tcctgcctga aggagctggt
ggcccgagtg ctgcagaggc tgtgcgagcg cggcgcgaag aacgtgctgg ccttcggctt
cgcgctgctg gacggggccc gcgggggccc ccccgaggcc ttcaccacca gcgtgcgcag
ctacctgccc aacacggtga ccgacgcact gcgggggagc ggggcgtggg ggctgctgct
gcgccgcgtg ggcgacgacg tgctggttca cctgctggca cgctgcgcgc tctttgtgct
ggtggctccc agctgcgcct accaggtgtg cgggccgccg ctgtaccagc tcggcgctgc
cactcaggcc cggcccccgc cacacgctag tggaccccga aggcgtctgg gatgcgaacg
ggcctggaac catagcgtca gggaggccgg ggtccccctg ggcctgccag ccccgggtgc
gaggaggcgc gggggcagtg ccagccgaag tctgccgttg cccaagaggc ccaggcgtgg
cgctgcccct gagccggagc ggacgcccgt tgggcagggg tectgggccc acccgggcag
gacgcgtgga ccgagtgacc gtggtttctg tgtggtgtca cctgccagac ccgccgaaga
agccacctct ttggagggtg cgctctctgg cacgcgccac toccacccat ccgtgggccg
ccagcaccac gcgggccccc catccacatc gcggccacca cgtccctggg acacgccttg
tcccccggtg tacgccgaga ccaagcactt cctctactcc tcaggcgaca aggagcagct
gcggccctcc ttcctactca gctctctgag gcccagcctg actggcgctc ggaggctcgt
ggagaccatc tttctgggtt ccaggccctg gatgccaggg actccccgca ggttgccccg
cctgccccag cgctactggc aaatgcggcc cctgtttctg gagctgcttg ggaaccacgc
gcagtgcccc tacggggtgc tcctcaagac gcactgcccg ctgcgagctg cggtcacccc
agcagccggt gtctgtgccc gggagaagcc ccagggctct gtggcggccc ccgaggagga
ggacacagac ccccgtcgcc tggtgcagct gctccgccag cacagcagcc cctggcaggt
gtacggcttc gtgcgggcct gcctgcgccg gctggtgccc ccaggcctct ggggctccag
gcacaacgaa cgccgcttcc tcaggaacac caagaagttc atctccctgg ggaagcatgc
caagctctcg ctgcaggagc tgacgtggaa gatgagcgtg cgggactgcg cttggctgcg
caggagccca ggggttggct gtgttccggc cgcagagcac cgtctgcgtg aggagatcct
ggccaagttc ctgcactggc tgatgagtgt gtacgtcgtc gagctgctca ggtctttctt
ttatgtcacg gagaccacgt ttcaaaagaa caggctcttt ttctaccgga agagtgtctg
gagcaagttg caaagcattg gaatcagaca gcacttgaag agggtgcagc tgcgggagct
gtcggaagca gaggtcaggc agcatcggga agccaggccc gccctgctga cgtccagact
ccgcttcatc cccaagcctg acgggctgcg gccgattgtg aacatggact acgtcgtggg
agccagaacg ttccgcagag aaaagagggc cgagcgtctc acctcgaggg tgaaggcact
gttcagcgtg ctcaactacg agcgggcgcg gcgccccggc ctcctgggcg cctctgtgct
gggcctggac gatatccaca gggcctggcg caccttcgtg ctgcgtgtgc gggcccagga
cccgccgcct gagctgtact ttgtcaaggt ggatgtgacg ggcgcgtacg acaccatccc
ccaggacagg ctcacggagg tcatcgccag catcatcaaa ccccagaaca cgtactgcgt
gcgtcggtat gccgtggtcc agaaggccgc ccatgggcac gtccgcaagg ccttcaagag
ccacgtctct accttgacag acctccagcc gtacatgcga cagttcgtgg ctcacctgca
ggagaccagc ccgctgaggg atgccgtcgt catcgagcag agctcctccc tgaatgaggc
cagcagtggc ctcttcgacg tcttcctacg cttcatgtgc caccacgccg tgcgcatcag
gggcaagtcc tacgtccagt gccaggggat cccgcagggc tccatcctct ccacgctgct
ctgcagcctg tgctacggcg acatggagaa caagctgttt gcggggattc ggcgggacgg
gctgctcctg cgtttggtgg atgatttett gttggtgaca cctcacctca cccacgcgaa
aaccttcctc aggaccctgg tccgaggtgt ccctgagtat ggctgcgtgg tgaacttgcg
gaagacagtg gtgaacttcc ctgtagaaga cgaggccctg ggtggcacgg cttttgttca
gatgccggcc cacggcctat tcccctggtg cggcctgctg ctggataccc ggaccctgga
ggtgcagagc gactactcca gctatgcccg gacctccatc agagccagtc tcaccttcaa
ccgcggcttc aaggctggga ggaacatgcg tcgcaaactc tttggggtct tgcggctgaa
gtgtcacagc ctgtttctgg atttgcaggt gaacagcctc cagacggtgt gcaccaacat
ctacaagatc ctcctgctgc aggcgtacag gtttcacgca tgtgtgctgc agctcccatt
tcatcagcaa gtttggaaga accccacatt tttcctgcgc gtcatctctg acacggcctc
cctctgctac tccatcctga aagccaagaa cgcagggatg tcgctggggg ccaagggcgc
cgccggccct ctgccctccg aggccgtgca gtggctgtgc caccaagcat tcctgctcaa
gctgactcga caccgtgtca cctacgtgcc actcctgggg tcactcagga cagcccagac
gcagctgagt cggaagctcc cggggacgac gctgactgcc ctggaggccg cagccaaccc
ggcactgccc tcagacttca agaccatcct ggactgatgg ccacccgccc acagccaggc
cgagagcaga caccagcagc cctgtcacgc cgggctctac gtcccaggga gggaggggcg
gcccacaccc aggcccgcac cgctgggagt ctgaggcctg agtgagtgtt tggccgaggc
ctgcatgtcc ggctgaaggc tgagtgtccg gctgaggcct gagcgagtgt ccagccaagg
gctgagtgtc cagcacacct gccgtcttca cttccccaca ggctggcgct cggctccacc
ccagggccag cttttcctca ccaggagccc ggcttccact ccccacatag gaatagtcca
tccccagatt cgccattgtt cacccctcgc cctgccctcc tttgccttcc acccccacca
tccaggtgga gaccctgaga aggaccctgg gagctctggg aatttggagt gaccaaaggt
gtgccctgta cacaggcgag gaccctgcac ctggatgggg gtccctgtgg gtcaaattgg
ggggaggtgc tgtgggagta aaatactgaa tatatgagtt tttcagtttt gaaaaaaa
SEQ ID NO: 2
MPRAPRCRAVRSLLRSHYREVLPLATFVRRLGPQGWRLVQRGDPAAFRALVAQCLVCVPWDARPPPAAPSFRQVS
CLKELVARVLQRLCERGAKNVLAFGFALLDGARGGPPEAFTTSVRSYLPNTVTDALRGSGAWGLLLRRVGDDVLV
HLLARCALFVLVAPSCAYQVCGPPLYQLGAATQARPPPHASGPRRRLGCERAWNHSVREAGVPLGLPAPGARRRG
GSASRSLPLPKRPRRGAAPEPERTPVGQGSWAHPGRTRGPSDRGFCVVSPARPAEEATSLEGALSGTRHSHPSVG
RQHHAGPPSTSRPPRPWDTPCPPVYAETKHFLYSSGDKEQLRPSFLLSSLRPSLTGARRLVETIFLGSRPWMPGT
PRRLPRLPQRYWQMRPLFLELLGNHAQCPYGVLLKTHCPLRAAVTPAAGVCAREKPQGSVAAPEEEDTDPRRLVQ
LLRQHSSPWQVYGFVRACLRRLVPPGLWGSRHNERRFLRNTKKFISLGKHAKLSLQELTWKMSVRDCAWLRRSPG
VGCVPAAEHRLREEILAKFLHWLMSVYVVELLRSFFYVTETTFQKNRLFFYRKSVWSKLQSIGIRQHLKRVQLRE
LSEAEVRQHREARPALLTSRLRFIPKPDGLRPIVNMDYVVGARTFRREKRAERLTSRVKALFSVLNYERARRPGL
LGASVLGLDDIHRAWRTFVLRVRAQDPPPELYFVKVDVTGAYDTIPQDRLTEVIASIIKPQNTYCVRRYAVVQKA
AHGHVRKAFKSHVSTLTDLQPYMRQFVAHLQETSPLRDAVVIEQSSSLNEASSGLEDVFLRFMCHHAVRIRGKSY
VQCQGIPQGSILSTLLCSLCYGDMENKLFAGIRRDGLLLRLVDDFLLVTPHLTHAKTFLRTLVRGVPEYGCVVNL
RKTVVNFPVEDEALGGTAFVQMPAHGLFPWCGLLLDTRTLEVQSDYSSYARTSIRASLTFNRGFKAGRNMRRKLF
GVLRLKCHSLFLDLQVNSLQTVCTNIYKILLLQAYRFHACVLQLPFHQQVWKNPTFFLRVISDTASLCYSILKAK
NAGMSLGAKGAAGPLPSEAVQWLCHQAFLLKLTRHRVTYVPLLGSLRTAQTQLSRKLPGTTLTALEAAANPALPS
DFKTILD
SEQ ID NO: 3
caggcagcgc tgcgtcctgc tgcgcacgtg ggaagccctg gccccggcca cccccgcgat
gccgcgcgct ccccgctgcc gagccgtgcg ctccctgctg cgcagccact accgcgaggt
gctgccgctg gccacgttcg tgcggcgcct ggggccccag ggctggcggc tggtgcagcg
cggggacccg gcggctttcc gcgcgctggt ggcccagtgc ctggtgtgcg tgccctggga
cgcacggccg ccccccgccg ccccctcctt ccgccaggtg tcctgcctga aggagctggt
ggcccgagtg ctgcagaggc tgtgcgagcg cggcgcgaag aacgtgctgg ccttcggctt
cgcgctgctg gacggggccc gcgggggccc ccccgaggcc ttcaccacca gcgtgcgcag
ctacctgccc aacacggtga ccgacgcact gcgggggagc ggggcgtggg ggctgctgct
gcgccgcgtg ggcgacgacg tgctggttca cctgctggca cgctgcgcgc tctttgtgct
ggtggctccc agctgcgcct accaggtgtg cgggccgccg ctgtaccagc tcggcgctgc
cactcaggcc cggcccccgc cacacgctag tggaccccga aggcgtctgg gatgcgaacg
ggcctggaac catagcgtca gggaggccgg ggtccccctg ggcctgccag ccccgggtgc
gaggaggcgc gggggcagtg ccagccgaag tctgccgttg cccaagaggc ccaggcgtgg
cgctgcccct gagccggagc ggacgcccgt tgggcagggg tectgggccc acccgggcag
gacgcgtgga ccgagtgacc gtggtttctg tgtggtgtca cctgccagac ccgccgaaga
agccacctct ttggagggtg cgctctctgg cacgcgccac tcccacccat ccgtgggccg
ccagcaccac gcgggccccc catccacatc gcggccacca cgtccctggg acacgccttg
tcccccggtg tacgccgaga ccaagcactt cctctactcc tcaggcgaca aggagcagct
gcggccctcc ttcctactca gctctctgag gcccagcctg actggcgctc ggaggctcgt
ggagaccatc tttctgggtt ccaggccctg gatgccaggg actccccgca ggttgccccg
cctgccccag cgctactggc aaatgcggcc cctgtttctg gagctgcttg ggaaccacgc
gcagtgcccc tacggggtgc tcctcaagac gcactgcccg ctgcgagctg cggtcacccc
agcagccggt gtctgtgccc gggagaagcc ccagggctct gtggcggccc ccgaggagga
ggacacagac ccccgtcgcc tggtgcagct gctccgccag cacagcagcc cctggcaggt
gtacggcttc gtgcgggcct gcctgcgccg gctggtgccc ccaggcctct ggggctccag
gcacaacgaa cgccgcttcc tcaggaacac caagaagttc atctccctgg ggaagcatgc
caagctctcg ctgcaggagc tgacgtggaa gatgagcgtg cgggactgcg cttggctgcg
caggagccca ggggttggct gtgttccggc cgcagagcac cgtctgcgtg aggagatcct
ggccaagttc ctgcactggc tgatgagtgt gtacgtcgtc gagctgctca ggtctttctt
ttatgtcacg gagaccacgt ttcaaaagaa caggctcttt ttctaccgga agagtgtctg
gagcaagttg caaagcattg gaatcagaca gcacttgaag agggtgcagc tgcgggagct
gtcggaagca gaggtcaggc agcatcggga agccaggccc gccctgctga cgtccagact
ccgcttcatc cccaagcctg acgggctgcg gccgattgtg aacatggact acgtcgtggg
agccagaacg ttccgcagag aaaagagggc cgagcgtctc acctcgaggg tgaaggcact
gttcagcgtg ctcaactacg agcgggcgcg gogccccggc ctcctgggcg cctctgtgct
gggcctggac gatatccaca gggcctggcg caccttcgtg ctgcgtgtgc gggcccagga
cccgccgcct gagctgtact ttgtcaagga caggctcacg gaggtcatcg ccagcatcat
caaaccccag aacacgtact gcgtgcgtcg gtatgccgtg gtccagaagg ccgcccatgg
gcacgtccgc aaggccttca agagccacgt ctctaccttg acagacctcc agccgtacat
gcgacagttc gtggctcacc tgcaggagac cagcccgctg agggatgccg tcgtcatcga
gcagagctcc tccctgaatg aggccagcag tggcctcttc gacgtcttcc tacgcttcat
gtgccaccac gccgtgcgca tcaggggcaa gtcctacgtc cagtgccagg ggatcccgca
gggctccatc ctctccacgc tgctctgcag cctgtgctac ggcgacatgg agaacaagct
gtttgcgggg attcggcggg acgggctgct cctgcgtttg gtggatgatt tcttgttggt
gacacctcac ctcacccacg cgaaaacctt cctcaggacc ctggtccgag gtgtccctga
gtatggctgc gtggtgaact tgcggaagac agtggtgaac ttccctgtag aagacgaggc
cctgggtggc acggcttttg ttcagatgcc ggcccacggc ctattcccct ggtgcggcct
gctgctggat acccggaccc tggaggtgca gagcgactac tccagctatg cccggacctc
catcagagcc agtctcacct tcaaccgcgg cttcaaggct gggaggaaca tgcgtcgcaa
actctttggg gtcttgcggc tgaagtgtca cagcctgttt ctggatttgc aggtgaacag
cctccagacg gtgtgcacca acatctacaa gatcctcctg ctgcaggcgt acaggtttca
cgcatgtgtg ctgcagctcc catttcatca gcaagtttgg aagaacccca catttttcct
gcgcgtcatc tctgacacgg cctccctctg ctactccatc ctgaaagcca agaacgcagg
gatgtcgctg ggggccaagg gcgccgccgg ccctctgccc tccgaggccg tgcagtggct
gtgccaccaa gcattcctgc tcaagctgac tcgacaccgt gtcacctacg tgccactcct
ggggtcactc aggacagccc agacgcagct gagtcggaag ctcccgggga cgacgctgac
tgccctggag gccgcagcca acccggcact gccctcagac ttcaagacca tcctggactg
atggccaccc gcccacagcc aggccgagag cagacaccag cagccctgtc acgccgggct
ctacgtccca gggagggagg ggcggcccac acccaggccc gcaccgctgg gagtctgagg
cctgagtgag tgtttggccg aggcctgcat gtccggctga aggctgagtg tccggctgag
gcctgagcga gtgtccagcc aagggctgag tgtccagcac acctgccgtc ttcacttccc
cacaggctgg cgctcggctc caccccaggg ccagcttttc ctcaccagga gcccggcttc
cactccccac ataggaatag tccatcccca gattcgccat tgttcacccc tcgccctgcc
ctcctttgcc ttccaccccc accatccagg tggagaccct gagaaggacc ctgggagctc
tgggaatttg gagtgaccaa aggtgtgccc tgtacacagg cgaggaccct gcacctggat
gggggtccct gtgggtcaaa ttggggggag gtgctgtggg agtaaaatac tgaatatatg
agtttttcag ttttgaaaaa aa
SEQ ID NO: 4
MPRAPRCRAVRSLLRSHYREVLPLATFVRRLGPQGWRLVQRGDPAAFRALVAQCLVCVPWDARPPPAAPSFRQVS
CLKELVARVLQRLCERGAKNVLAFGFALLDGARGGPPEAFTTSVRSYLPNTVTDALRGSGAWGLLLRRVGDDVLV
HLLARCALFVLVAPSCAYQVCGPPLYQLGAATQARPPPHASGPRRRLGCERAWNHSVREAGVPLGLPAPGARRRG
GSASRSLPLPKRPRRGAAPEPERTPVGQGSWAHPGRTRGPSDRGFCVVSPARPAEEATSLEGALSGTRHSHPSVG
RQHHAGPPSTSRPPRPWDTPCPPVYAETKHFLYSSGDKEQLRPSFLLSSLRPSLTGARRLVETIFLGSRPWMPGT
PRRLPRLPQRYWQMRPLFLELLGNHAQCPYGVLLKTHCPLRAAVTPAAGVCAREKPQGSVAAPEEEDTDPRRLVQ
LLRQHSSPWQVYGFVRACLRRLVPPGLWGSRHNERRFLRNTKKFISLGKHAKLSLQELTWKMSVRDCAWLRRSPG
VGCVPAAEHRLREEILAKFLHWLMSVYVVELLRSFFYVTETTFQKNRLFFYRKSVWSKLQSIGIRQHLKRVQLRE
LSEAEVRQHREARPALLTSRLRFIPKPDGLRPIVNMDYVVGARTFRREKRAERLTSRVKALFSVLNYERARRPGL
LGASVLGLDDIHRAWRTFVLRVRAQDPPPELYFVKDRLTEVIASIIKPQNTYCVRRYAVVQKAAHGHVRKAFKSH
VSTLTDLQPYMRQFVAHLQETSPLRDAVVIEQSSSLNEASSGLEDVFLRFMCHHAVRIRGKSYVQCQGIPQGSIL
STLLCSLCYGDMENKLFAGIRRDGLLLRLVDDFLLVTPHLTHAKTFLRTLVRGVPEYGCVVNLRKTVVNFPVEDE
ALGGTAFVQMPAHGLFPWCGLLLDTRTLEVQSDYSSYARTSIRASLTFNRGFKAGRNMRRKLFGVLRLKCHSLFL
DLQVNSLQTVCTNIYKILLLQAYRFHACVLQLPFHQQVWKNPTFFLRVISDTASLCYSILKAKNAGMSLGAKGAA
GPLPSEAVQWLCHQAFLLKLTRHRVTYVPLLGSLRTAQTQLSRKLPGTTLTALEAAANPALPSDFKTILD
SEQ ID NO: 5
gctctctgct gcgcagccga taccgggagg tgtggccgct ggcaaccttt gtgcggcgcc
tggggcccga gggcaggcgg cttgtgcaac ccggggaccc gaagatctac cgcactttgg
ttgcccaatg cctagtgtgc atgcactggg gctcacagcc tccacctgcc gacctttcct
tccaccaggt gtcatccctg aaagagctgg tggccagggt tgtgcagaga ctctgcgagc
gcaacgagag aaacgtgctg gcttttggct ttgagctgct taacgaggcc agaggcgggc
ctcccatggc cttcactagt agcgtgcgta gctacttgcc caacactgtt attgagaccc
tgcgtgtcag tggtgcatgg atgctactgt tgagccgagt gggcgacgac ctgctggtct
acctgctggc acactgtgct ctttatcttc tggtgccccc cagctgtgcc taccaggtgt
gtgggtctcc cctgtaccaa atttgtgcca ccacggatat ctggccctct gtgtccgcta
gttacaggcc cacccgaccc gtgggcagga atttcactaa ccttaggttc ttacaacaga
tcaagagcag tagtcgccag gaagcaccga aacccctggc cttgccatct cgaggtacaa
agaggcatct gagtctcacc agtacaagtg tgccttcagc taagaaggcc agatgctatc
ctgtcccgag agtggaggag ggaccccaca ggcaggtgct accaacccca tcaggcaaat
catgggtgcc aagtcctgct cggtcccccg aggtgcctac tgcagagaaa gatttgtctt
ctaaaggaaa ggtgtctgac ctgagtctct ctgggtcggt gtgctgtaaa cacaagccca
gctccacatc tctgctgtca ccaccccgcc aaaatgcctt tcagctcagg ccatttattg
agaccagaca tttcctttac tccaggggag atggccaaga gcgtctaaac ccctcattcc
tactcagcaa cctccagcct aacttgactg gggccaggag actggtggag atcatctttc
tgggctcaag gcctaggaca tcaggaccac tctgcaggac acaccgtcta tcgcgtcgat
actggcagat gcggcccctg ttccaacage tgctggtgaa ccatgcagag tgccaatatg
tcagactcct caggtcacat tgcaggtttc gaacagcaaa ccaacaggtg acagatgcct
tgaacaccag cccaccgcac ctcatggatt tgctccgcct gcacagcagt ccctggcagg
tatatggttt tcttcgggcc tgtctctgca aggtggtgtc tgctagtctc tggggtacca
ggcacaatga gcgccgcttc tttaagaact taaagaagtt catctcgttg gggaaatacg
gcaagctatc actgcaggaa ctgatgtgga agatgaaagt agaggattgc cactggctcc
gcagcagccc ggggaaggac cgtgtccccg ctgcagagca ccgtctgagg gagaggatcc
tggctacgtt cctgttctgg ctgatggaca catacgtggt acagctgctt aggtcattct
tttacatcac agagagcaca ttccagaaga acaggctctt cttctaccgt aagagtgtgt
ggagcaagct gcagagcatt ggagtcaggc aacaccttga gagagtgcgg ctacgggagc
tgtcacaaga ggaggtcagg catcaccagg acacctggct agccatgccc atctgcagac
tgcgcttcat ccccaagccc aacggcctgc ggcccattgt gaacatgagt tatagcatgg
gtaccagagc tttgggcaga aggaagcagg cccagcattt cacccagcgt ctcaagactc
tcttcagcat gctcaactat gagcggacaa aacatcctca ccttatgggg tcttctgtac
tgggtatgaa tgacatctac aggacctggc gggcctttgt gctgcgtgtg cgtgctctgg
accagacacc caggatgtac tttgttaagg cagatgtgac cggggcctat gatgccatcc
cccagggtaa gctggtggag gttgttgcca atatgatcag gcactcggag agcacgtact
gtatccgcca gtatgcagtg gtccggagag atagccaagg ccaagtccac aagtccttta
ggagacaggt caccaccctc tctgacctcc agccatacat gggccagttc cttaagcatc
tgcaggattc agatgccagt gcactgagga actccgttgt catcgagcag agcatctcta
tgaatgagag cagcagcagc ctgtttgact tcttcctgca cttcctgcgt cacagtgtcg
taaagattgg tgacaggtgc tatacgcagt gccagggcat cccccagggc tccagcctat
ccaccctgct ctgcagtctg tgtttcggag acatggagaa caagctgttt gctgaggtgc
agcgggatgg gttgctttta cgttttgttg atgactttct gttggtgacg cctcacttgg
accaagcaaa aaccttcctc agcaccctgg tccatggcgt tcctgagtat gggtgcatga
taaacttgca gaagacagtg gtgaacttcc ctgtggagcc tggtaccctg ggtggtgcag
ctccatacca gctgcctgct cactgcctgt ttccctggtg tggcttgctg ctggacactc
agactttgga ggtgttctgt gactactcag gttatgccca gacctcaatt aagacgagcc
tcaccttcca gagtgtcttc aaagctggga agaccatgeg gaacaagctc ctgtcggtct
tgcggttgaa gtgtcacggt ctatttctag acttgcaggt gaacagcctc cagacagtct
gcatcaatat atacaagatc ttcctgcttc aggcctacag gttccatgca tgtgtgattc
agcttccctt tgaccagcgt gttaggaaga acctcacatt ctttctgggc atcatctcca
gccaagcatc ctgctgctat gctatcctga aggtcaagaa tccaggaatg acactaaagg
cctctggctc ctttcctect gaagccgcac attggctctg ctaccaggcc ttcctgctca
agctggctgc tcattctgtc atctacaaat gtctcctggg acctctgagg acagcccaaa
aactgctgtg ccggaagctc ccagaggcga caatgaccat ccttaaagct gcagctgacc
cagccctaag cacagacttt cagaccattt tggactaacc ctgtctcctt ccgctagatg
aacatg

| | |
|---|---|
| SEQ ID NO: 6 | Acta2 F ATCACCAACTGGGACGACAT |
| SEQ ID NO: 7 | Acta2 R AGTGTCGGATGCTCTTCAGG |
| SEQ ID NO: 8 | Vim F CATGATGCTTTGGGTAAAATGG |
| SEQ ID NO: 9 | Vim R AGTGGGTGTCAACCAGAGGA |
| SEQ ID NO: 10 | Col1a1 F TGGAATCATGGTATTTGGAACA |
| SEQ ID NO: 11 | Col1a1 R TAGTCGTATGCTGCCTTGGG |
| SEQ ID NO: 12 | Col1a2 F CAGGGCCTGATGGAAACAA |
| SEQ ID NO: 13 | Col1a2 R ACCCCTCTCTCCTGGAAGC |
| SEQ ID NO: 14 | Col3a1 F ATAATGGGGAACGTGGTCCT |
| SEQ ID NO: 15 | Col3a1 R TGACCATCTGATCCAGGGTT |
| SEQ ID NO: 16 | Col4a1 F GTCTGGCTTCTGCTGCTCTTC |
| SEQ ID NO: 17 | Col4a1 R TCACATTTTCCACAGCCAGA |
| SEQ ID NO: 18 | Fn1 F CCTGGGAGAAGTTTGTGCAT |
| SEQ ID NO: 19 | Fn1 R ACTTGGACAGGTCCAGTTGTG |
| SEQ ID NO: 20 | Emr1 F GCACCATCTTGCTGGAGACT |
| SEQ ID NO: 21 | Emr1 R TTCATTGATGACTTTGCTTTCG |
| SEQ ID NO: 22 | Tgfb1 F GGAGAGCCCTGGATACCAAC |
| SEQ ID NO: 23 | Tgfb1 R ATCCACTTCCAACCCAGGTC |
| SEQ ID NO: 24 | Snai1 F ACCCTGCTGGTATCTCTCCC |
| SEQ ID NO: 25 | Snai1 R TCTTCACATCCGAGTGGGTT |
| SEQ ID NO: 26 | Snai2 F GGCTGCTTCAAGGACACATT |
| SEQ ID NO: 27 | Snai2 R GTGCCCTCAGGTTTGATCTG |
| SEQ ID NO: 28 | Zeb1 F CCAAGAACTGCTGGCAAGA |
| SEQ ID NO: 29 | Zeb1 R TTCGGATCATGGTTTTGCTC |
| SEQ ID NO: 30 | Zeb2 F AAACAAGCCAATCCCAGGAG |
| SEQ ID NO: 31 | Zeb2 R GAGGGTTTGCAAGGCTATCA |
| SEQ ID NO: 32 | Twist F CACGCTGCCCTCGGACAA |
| SEQ ID NO: 33 | Twist R GGGACGCGGACATGGACC |
| SEQ ID NO: 34 | Kim-1 F CTCTACCTCCACTCCTCCAACA |
| SEQ ID NO: 35 | Kim-1 R CTCCTGAGGATGTCACAGTGC |
| SEQ ID NO: 36 | NGAL F CAATGTCACCTCCATCCTGG |
| SEQ ID NO: 37 | NGAL R CCTGGAGCTTGGAACAAATG |
| SEQ ID NO: 38 | Smad3 F TGAACACCAAGTGCATTACCA |
| SEQ ID NO: 39 | Smad3 R AGGCGGCAGTAGATAACGTG |
| SEQ ID NO: 40 | Cdh1 F GCAGAACTGTCCCTGTCCCAG |
| SEQ ID NO: 41 | Cdh1 R GAACAGCACGTACACAGCCCT |
| SEQ ID NO: 42 | Loxl2 F CCTACAACCCCAAAGCCTATAA |
| SEQ ID NO: 43 | Loxl2 R CGTGCAGTTCATAGAAAACTTCC |
| SEQ ID NO: 44 | GAPDH F AGGTCGGTGTGAACGGATTTG |
| SEQ ID NO: 45 | GAPDH R TGTAGACCATGTAGTTGAGGTCA |
| SEQ ID NO: 46 | Trf1 F TGGAAAATGAGAAAGCGAGG |
| SEQ ID NO: 47 | Trf1 R TTTCTGTAGACTGCTGGCCATT |
| SEQ ID NO: 48 | mTERT F GGTGACAGATGCCTTGAACAC |
| SEQ ID NO: 49 | mTERT R GATCCTCTCCCTCAGACGGT |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### EXAMPLES

### Example 1: Methods

### Mice and Animal Procedures

Tert heterozygous mice generated as previously described63 were backcrossed to a >98% C57BL/6 background. Tert^{+/-} mice were intercrossed to generate first generation (G1) homozygous Tert^{-/-} knockout mice. G3 Tert^{-/-} mice were generated by successive breeding of G2 Tert^{-/-} mice. Tert^{+/+} and G3 Tert^{-/-} male and female mice of pure C57BL/6 background were treated with FA (F7876; Sigma-Aldrich). Male and female G3 Tert^{-/-} mice (aged 7, 27 and 47 weeks) were euthanized to analyze the kidney phenotype for signs of fibrosis and for RNA-seq analysis.

Trf1^{lox/lox} mice64 were crossed with mice expressing CreERT2 recombinase driven by the tamoxifen-inducible ubiquitin C (UBC) promoter65 to generate Trf1^{lox/lox};hUBC-CreERT2 or Trf1^{+/+};hUBC-CreERT2 mice. These mice were fed ad libitum with a long-term tamoxifen-containing diet, starting at 10 weeks of age.

All mice were generated and maintained at the Animal Facility of the Spanish National Cancer Research Centre (CNIO) under specific pathogen-free conditions with a 12-h light/dark cycle. Mice were housed in plastic cages containing wood shavings or chipped wood bedding with food and water available ad libitum in accordance with the recommendation of the Federation of European Laboratory Animal Science Associations. All animal procedures were approved by the CNIO-ISCIII Ethics Committee for Research and Animal Welfare (CBA 03_2019-v2).

### Folic Acid Dose Titration and Inoculation

A single intraperitoneal injection of FA with different doses (50, 100, 125 and 250 mg kg⁻¹ body weight) in vehicle (0.2 ml of 0.3 mmol l⁻¹ NaHCO₃) or vehicle alone was administered to 6- to 8-week-old Tert^{+/+} and G3 Tert^{-/-} mice. Blood was collected at days 7 and 14 and was analyzed by VetScan Comprehensive Diagnostic Profile kit. Mice were euthanized at day 14 after FA injection, and the kidneys were collected from FA-treated or vehicle-treated animals and analyzed by immunohistochemistry for kidney fibrosis.

A single intraperitoneal injection of FA with a low dose of 125 mg kg⁻¹ body weight or 0.3 M NaHCO₃ (200 ul) was inoculated to 6- to 8-week-old Tert^{+/+} and G3 Tert^{-/-} mice. Blood collected at days 2, 7 and 14 and was analyzed by VetScan Comprehensive Diagnostic Profile kit. Mice were euthanized at day 14, and kidneys were perfused with cold PBS and harvested.

### Morphological Analyses

Kidneys were fixed in 4% formaldehyde and embedded in paraffin. Paraffin sections (5-μm thick) were stained with Masson's trichrome, Sirius red and PAS+D using standard procedures. Percentages of fibrotic areas were quantified using the National Institutes of Health (NIH) ImageJ program.

### Primary Cell Culture

Primary mouse PTCs were isolated from 10- to 11-week-old Tert^{+/+} and G3 Tert^{-/-} mice as described previously66. mTert-pBabe-puro was a gift from M. Alvarez and J. Bidwell (Addgene plasmid no. 36413). Retroviral plasmid vectors (mTert-pBabe-puro) and the packaging plasmids (PLC Eco and pCMV-VSV-G) were co-transfected into the packaging cell line 293T. Viral supernatants were collected 48 h later, centrifuged to remove cell debris, strained through 0.45-μm filters (Millipore) and used to infect isolated PTCs from Tert^{+/+} and G3 Tert^{-/-} mice. Stable cell lines were selected with 2 μg ml⁻¹ puromycin on day 10 until day 14. Cells were collected for RNA extraction and immunofluorescence.

### Immunohistochemical and Immunofluorescence Staining

Kidney tissues were fixed in 4% formaldehyde and embedded in paraffin. Paraffin-embedded kidney tissue sections at a thickness of 5-7 μm were subjected to immunohistochemical staining. Primary antibodies (and their dilutions) were: rat monoclonal to p21 (HUGO-291H/B5; CNIO histopathology core unit; 1:400), rat monoclonal to p53 (POE316A/E9; CNIO histopathology core unit; 1:400), rat monoclonal to p21 (HUGO-291H/B5; CNIO histopathology core unit; 1:400) rat monoclonal to CD8a (AM-OTO94A; CNIO histopathology core unit), mouse monoclonal to phospho-Histone H2AX (Ser 139; 05-636; Millipore; 1:400), mouse monoclonal to E-cadherin (610182; BD Biosciences; 1:400), rat monoclonal to F4/80 (MCA497; AbD Serotec; 1:400), rabbit polyclonal activated caspase-3 (9661; Cell Signaling Technology; 1:400), rabbit monoclonal to CD3e (99940; Cell Signaling Technology; 1:400), rabbit monoclonal to CD4 (25229; Cell Signaling Technology; 1:400), rabbit polyclonal to Collagen type VI (ab6588; Abcam; 1:400), rabbit polyclonal to fibronectin (ab2413; Abcam; 1:400) and rabbit polyclonal to Sox9 (AB5535; Millipore; 1:400).

For immunofluorescence, mouse monoclonal antibody to α-SMA-Cy3 (C6198; Sigma; 1:400), rabbit monoclonal to vimentin (5741; Cell Signaling Technology; 1:200), rabbit monoclonal to Ki67 (12202; Cell Signaling Technology; 1:400), rabbit polyclonal TGFβ (3711S; Cell Signaling Technology; 1:200), rabbit polyclonal SNAIL+SLUG (ab180714; Abcam; 1:200) and rat monoclonal E-cadherin (DECMA-1; ab11512, Abcam; 1:200) were used. Images were obtained using a confocal ultra-spectral microscope (Leica TCS-SP5). The percentages of positively stained areas by immunohistochemistry and immunofluorescence were quantified using NIH ImageJ (v1.52n).

### Gene Expression Analysis and Real-Time PCR Examination

Total RNA was isolated from kidney tissues and PTCs using TRIzol reagent (Takara), according to the manufacturer's instructions. cDNA was synthesized with 1 μg of total RNA, cDNA synthesis mix (BioMake), and oligo-dT primers. Gene expression was measured by a real-time PCR assay (BioMake) and a 7900HT real-time PCR system (Applied Biosystems). The relative amount of mRNA to the internal control was calculated as 2^{ΔCT}, in which ΔCT=ΔCTₑₓₚₑᵣᵢₘₑₙₜₐₗ−ΔCT_{control}. Genes and primers are listed in Table 2 (F: forward primer; R: reverse primer).

TABLE 2
qPCR primers used in this study (sequence (5′-3′)

| | |
|---|---|
| SEQ ID NO: 6 | Acta2 F ATCACCAACTGGGACGACAT |
| SEQ ID NO: 7 | Acta2 R AGTGTCGGATGCTCTTCAGG |
| SEQ ID NO: 8 | Vim F CATGATGCTTTGGGTAAAATGG |
| SEQ ID NO: 9 | Vim R AGTGGGTGTCAACCAGAGGA |
| SEQ ID NO: 10 | Col1a1 F TGGAATCATGGTATTTGGAACA |
| SEQ ID NO: 11 | Col1a1 R TAGTCGTATGCTGCCTTGGG |
| SEQ ID NO: 12 | Col1a2 F CAGGGCCTGATGGAAACAA |
| SEQ ID NO: 13 | Col1a2 R ACCCCTCTCTCCTGGAAGC |
| SEQ ID NO: 14 | Col3a1 F ATAATGGGGAACGTGGTCCT |
| SEQ ID NO: 15 | Col3a1 R TGACCATCTGATCCAGGGTT |
| SEQ ID NO: 16 | Col4a1 F GTCTGGCTTCTGCTGCTCTTC |
| SEQ ID NO: 17 | Col4a1 R TCACATTTTCCACAGCCAGA |
| SEQ ID NO: 18 | Fn1 F CCTGGGAGAAGTTTGTGCAT |
| SEQ ID NO: 19 | Fn1 R ACTTGGACAGGTCCAGTTGTG |
| SEQ ID NO: 20 | Emr1 F GCACCATCTTGCTGGAGACT |
| SEQ ID NO: 21 | Emr1 R TTCATTGATGACTTTGCTTTCG |
| SEQ ID NO: 22 | Tgfb1 F GGAGAGCCCTGGATACCAAC |
| SEQ ID NO: 23 | Tgfb1 R ATCCACTTCCAACCCAGGTC |
| SEQ ID NO: 24 | Snai1 F ACCCTGCTGGTATCTCTCCC |
| SEQ ID NO: 25 | Snai1 R TCTTCACATCCGAGTGGGTT |
| SEQ ID NO: 26 | Snai2 F GGCTGCTTCAAGGACACATT |
| SEQ ID NO: 27 | Snai2 R GTGCCCTCAGGTTTGATCTG |
| SEQ ID NO: 28 | Zeb1 F CCAAGAACTGCTGGCAAGA |
| SEQ ID NO: 29 | Zeb1 R TTCGGATCATGGTTTTGCTC |
| SEQ ID NO: 30 | Zeb2 F AAACAAGCCAATCCCAGGAG |
| SEQ ID NO: 31 | Zeb2 R GAGGGTTTGCAAGGCTATCA |
| SEQ ID NO: 32 | Twist F CACGCTGCCCTCGGACAA |
| SEQ ID NO: 33 | Twist R GGGACGCGGACATGGACC |
| SEQ ID NO: 34 | Kim-1 F CTCTACCTCCACTCCTCCAACA |
| SEQ ID NO: 35 | Kim-1 R CTCCTGAGGATGTCACAGTGC |
| SEQ ID NO: 36 | NGAL F CAATGTCACCTCCATCCTGG |
| SEQ ID NO: 37 | NGAL R CCTGGAGCTTGGAACAAATG |
| SEQ ID NO: 38 | Smad3 F TGAACACCAAGTGCATTACCA |
| SEQ ID NO: 39 | Smad3 R AGGCGGCAGTAGATAACGTG |
| SEQ ID NO: 40 | Cdh1 F GCAGAACTGTCCCTGTCCCAG |
| SEQ ID NO: 41 | Cdh1 R GAACAGCACGTACACAGCCCT |
| SEQ ID NO: 42 | Loxl2 F CCTACAACCCCAAAGCCTATAA |
| SEQ ID NO: 43 | Loxl2 R CGTGCAGTTCATAGAAAACTTCC |
| SEQ ID NO: 44 | GAPDH F AGGTCGGTGTGAACGGATTTG |
| SEQ ID NO: 45 | GAPDH R TGTAGACCATGTAGTTGAGGTCA |
| SEQ ID NO: 46 | Trf1 F TGGAAAATGAGAAAGCGAGG |
| SEQ ID NO: 47 | Trf1 R TTTCTGTAGACTGCTGGCCATT |
| SEQ ID NO: 48 | mTERT F GGTGACAGATGCCTTGAACAC |
| SEQ ID NO: 49 | mTERT R GATCCTCTCCCTCAGACGGT |

For RNA-seq experiments, total RNA samples (300 ng) were used. RNA quality scores were 6.3 on average (range 4.7-8.0) when assayed on a PerkinElmer LabChip analyzer. Sequencing libraries were prepared with the QuantSeq 3′ mRNA-Seq Library Prep Kit (FWD) for Illumina (Lexogen; 015), following the manufacturer's instructions. Library generation was initiated by reverse transcription with oligo-dT priming, and a second-strand synthesis was performed from random primers by a DNA polymerase. Primers from both steps contained Illumina-compatible sequences. cDNA libraries were purified, applied to an Illumina flow cell for cluster generation and sequenced on an Illumina NextSeq 550 (with v2.5 reagent kits), following the manufacturer's protocols. Read adaptors and poly(A) tails were removed with the command ‘bbduk.sh’, following the Lexogen recommendations. Processed reads were analyzed with the Nextpresso pipeline67 as follows. Sequencing quality was checked with FastQC v0.11.7 (https://www.bioinformatics.babraham.ac.uk/projects/fastqc/). Reads were aligned to the mouse reference genome (GRCm38) with TopHat (v2.0.10)68 using Bowtie (v1.0.0)69 and SAMtools (v0.1.19)70 (--library-type fr-secondstrand in TopHat), allowing three mismatches and twenty multihits. Read counts were obtained with HTSeq-count (v0.6.1)71 using the mouse gene annotation from GENCODE (GRCm38; vM20 Ensembl 95). GSEAPreranked72 was used to perform GSEA for several gene signatures on a preranked gene list, setting 1,000 gene-set permutations. Only those gene sets with significant enrichment levels (FDR q value <0.25) were considered.

### Telomere Analysis

Q-FISH determination on paraffin-embedded tissue sections was performed as described previously41. After deparaffinization, tissues were postfixed in 4% formaldehyde for 5 min, washed three times for 5 min each in PBS and incubated at 37° C. for 15 min in pepsin solution (0.1% porcine pepsin, Sigma; 0.01 M HCl, Merck). After another round of washes and fixation as mentioned above, slides were dehydrated in an ethanol series (70%, 90% and 100%; 5 min each). After 10 min of air drying, 30 l of telomere probe mix (10 mM Tris-CI (pH 7), 25 mM MgCl₂, 9 mM citric acid, 82 mM Na₂HPO₄, 70% deionized formamide (Sigma), 0.25% blocking reagent (Roche) and 0.5 μg ml⁻¹ telomeric PNA probe (Panagene) were added to each slide. A coverslip was added and slides were incubated for 3 min at 85° C., and for a further 2 h at room temperature in a wet chamber in the dark. Slides were washed twice for 15 min each in 10 mM Tris-CI (pH 7), 0.1% BSA in 70% formamide under vigorous shaking, then three times for 5 min each in TBS 0.08% with Tween 20 and then incubated in a DAPI bath (4 μg ml⁻¹ in PBS; Sigma) before mounting samples in Vectashield medium (Vector). Confocal images were acquired as stacks every 1 μm for a total of 5 μm using a Leica SP5-MP confocal microscope, and maximum projections were done with LAS-AF software. Telomere signal intensity was quantified using Definiens software.

### Statistics and Reproducibility

No statistical methods were used to predetermine sample sizes, but our sample sizes are similar to those reported previously446. Mice were randomly allocated within the groups, and investigators were blinded to group allocation. Our sample sizes corresponded to the number of mice used for each experiment, as indicated. The quantitative analyses of immunohistochemistry stainings were performed in the whole scanned kidney section comprising 10-15 areas. For immunofluorescence analysis, 10-20 images were collected from each individual. Data distribution was assumed to be normal, but this was not formally tested. No data points or mice were excluded from the analyses. Results of statistical analyses are expressed as means±s.e.m. Statistical analyses of immunohistochemical/immunofluorescence quantifications and of qPCR and Q-FISH analyses were performed using one-way ANOVA with post hoc Tukey's test in Prism (GraphPad). Two-way ANOVA with post hoc Bonferroni's test was used to analyze the blood and urine parameters. Statistical significance was defined as P<0.05.

### Example 2: Results

A Mouse Model of Kidney Fibrosis is Associated with Short Telomeres

To test the involvement of short telomeres in kidney fibrosis, we first analyzed the kidneys of wild-type mice, as well as mice lacking the telomerase catalytic subunit, Tert, that were bred for three generations (G3) to induce the presence of very short telomeres, that is, G3 Tert^{-/-} mice24 (FIG. 9a). Both wild-type (Tert^{+/+}) and G3 Tert^{-/-} mice aged 8-9 weeks displayed grossly normal kidney histology, with no evidence of glomerular or tubular defects and with no fibrosis or accumulation of collagen fibers as determined by Masson's trichrome and Sirius red staining, respectively (FIG. 9b,c). Similarly, we did not detect increased tubular injury or loss of brush borders in the kidney as determined by periodic acid-Schiff with diastase staining (PAS+D; FIG. 9d). Also, in agreement with grossly normal kidneys in the G3 Tert^{-/-} mice, we did not see differences in the presence of activated fibroblasts, as determined by expression of smooth muscle actin (α-SMA); in apoptosis, as determined by expression of cleaved caspase-3 (CC3); in senescence, as determined by expression of the p21 cell cycle inhibitor; or in the expression of E-cadherin (a marker of EMT, associated with tissue fibrosis), compared with age-matched Tert^{+/+} mice (FIG. 9e-h).

The incidence of kidney fibrosis increases with age; therefore, it is likely to be caused by the combination of molecular and cellular aging events, such as the presence of short telomeres14 together with exogenous damage to the kidney. Thus, we next challenged the kidneys of Tert^{+/+} and G3 Tert^{-/-} mice with FA, previously described to induce kidney fibrosis at a high dose of 250 mg kg⁻¹ body weight27. FA-induced nephropathy is widely used to study interstitial kidney fibrosis4,27,28. In particular, FA administered intraperitoneally in mice leads to rapid appearance of FA crystals in tubules, followed by severe nephrotoxicity.

To this end, we first subjected Tert^{+/+} and G3 Tert^{-/-} mice aged 8-9 weeks to increasing doses of FA (50, 100, 125 and 250 mg kg⁻¹ body weight; FIG. 1a) and selected the highest dose of FA that did not induce kidney fibrosis in wild-type mice. Wild-type mice treated with 50, 100 and 125 mg kg⁻¹ body weight appeared well and did not show any signs of kidney pathology as determined by normal kidney appearance (FIG. 1b), normal creatinine and blood urea nitrogen (BUN) in the blood (FIG. 1c,d), as well as by absence of fibrosis as indicated by Masson's Trichrome staining (FIG. 1e) and absence of tubular injury by PAS+D staining (FIG. 1e). In contrast, the majority of wild-type mice (75%) treated with an FA dose of 250 mg kg⁻¹ body weight died at day 2 after treatment due to FA-induced acute renal failure (data not shown). Thus, 125 mg kg⁻¹ was the maximum tolerated dose of FA that did not cause lethality in wild-type mice with normal telomere length. We reasoned that this dose of FA, although not sufficient to induce kidney fibrosis in Tert^{+/+} mice, may synergize with short telomeres in telomerase-deficient mice to induce kidney fibrosis.

Indeed, we treated G3 Tert^{-/-} mice with the same doses of FA and observed that, even after the 125 mg kg⁻¹ dose, the kidneys presented a pale color (FIG. 1b), suggestive of kidney fibrosis. Indeed, G3 Tert^{-/-} mice treated with increasing doses of FA showed increased creatinine (FIG. 1c) and BUN (FIG. 1d) levels in the blood at a 125 mg kg⁻¹ FA dose, indicative of kidney failure, as well as increased fibrosis as detected by Masson's trichrome staining (FIG. 1e) and increased renal tubular injury as determined by increased PAS+D staining (FIG. 1e). FA doses below 125 mg kg⁻¹ did not induce a fibrotic phenotype in G3 Tert^{-/-} mice (FIG. 1e). Thus, we selected a dose of 125 mg kg⁻¹ body weight of FA for further experiments.

Severe Kidney Dysfunction in Telomerase-Deficient Mice Treated with a Sublethal Dose of Folic Acid

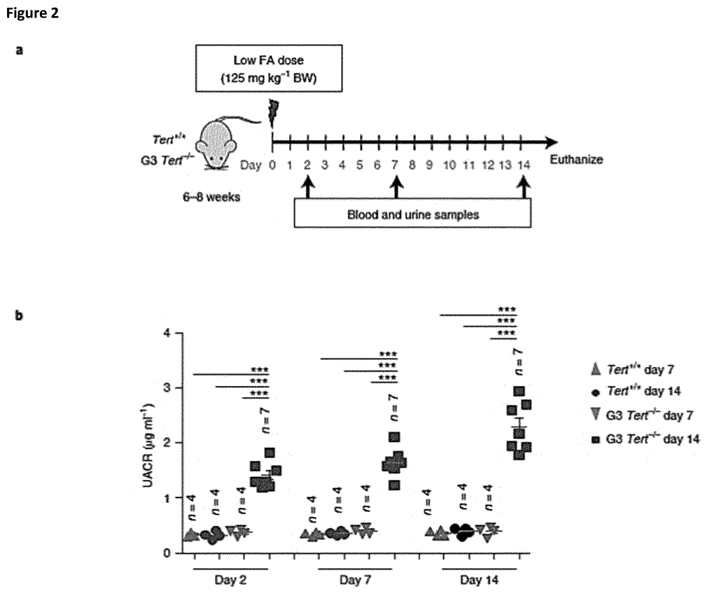

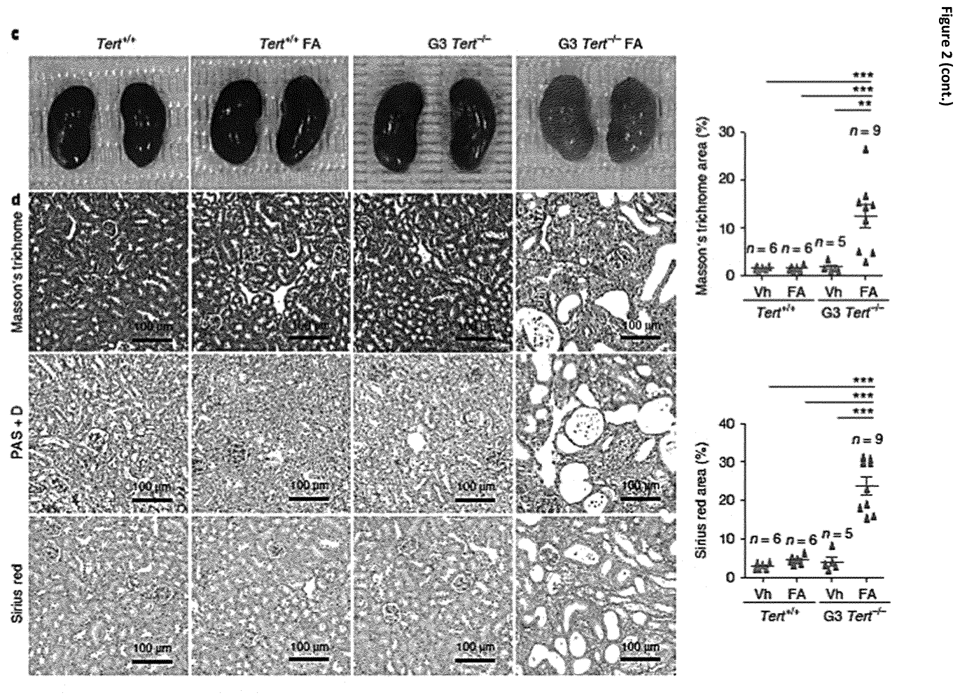

To address the role of short telomeres in kidney fibrosis, we subjected Tert^{+/+} and G3 Tert^{-/-} mice at 8-9 weeks of age to a dose of 125 mg kg⁻¹ body weight of FA, which does not induce fibrosis in wild-type mice (FIG. 2a). Blood was collected at days 2, 7 and 14 from the submandibular vein. At day 14, mice were euthanized and kidneys were harvested for further analysis. As expected, the kidneys from FA-treated G3 Tert^{-/-} mice appeared pale and damaged compared to similarly treated wild-type kidneys (FIG. 2b,c). To confirm kidney damage, we analyzed the urinary parameters and observed a marked increase in 24-h urinary albumin excretion and urinary albumin to creatinine ratio in FA-treated G3 Tert^{-/-} mice compared to FA-treated Tert^{+/+} and untreated Tert^{+/+} mice and G3 Tert^{-/-} mice (FIG. 2b).

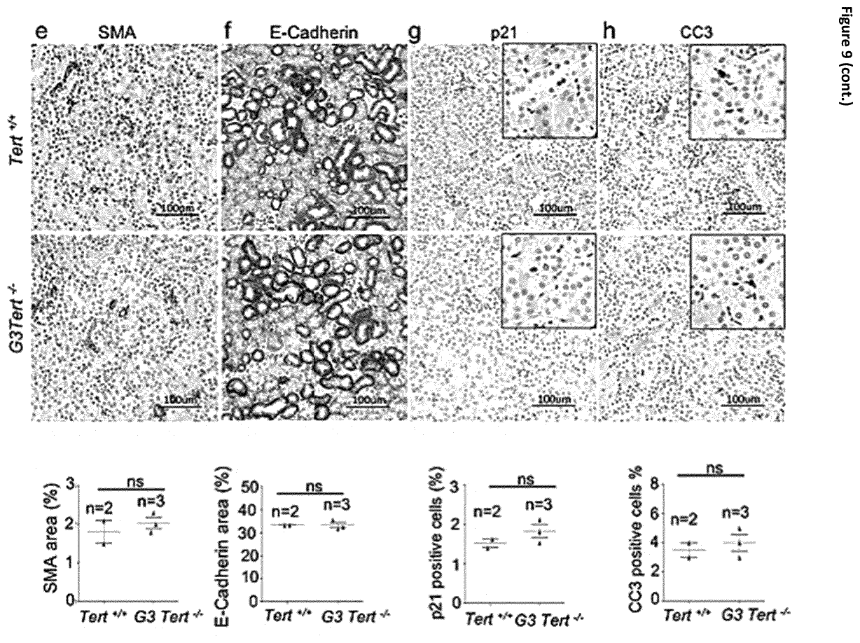

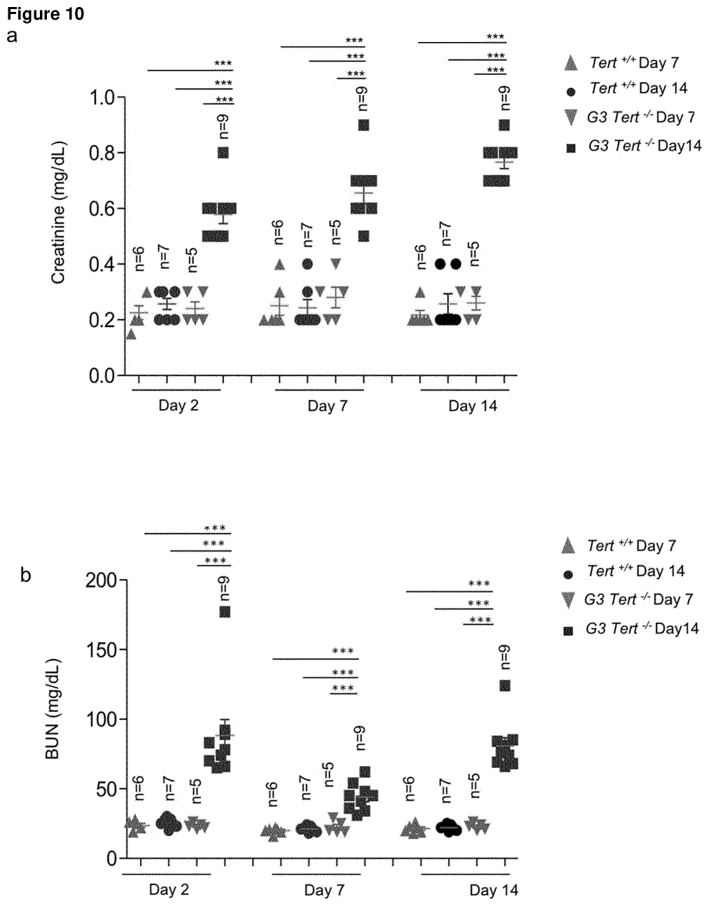

We also examined BUN and creatinine in all mouse cohorts. Administration of a high dose of FA is known to induce a transient elevation of BUN and creatinine levels at 48 h after injection followed by subsequent renal dysfunction accompanied with interstitial fibrosis29. Two days after the injection of a low dose of FA (125 mg kg⁻¹ body weight), however, both the untreated and FA-treated wild-type mice (Tert^{+/+}) mice showed normal BUN and creatinine levels, indicating normal kidney function (FIG. 10a,b). Consistently, we did not observe any reduced mobility or increased mortality of the wild-type mice (Tert^{+/+}) treated with a low dose of FA (125 mg kg⁻¹ body weight). Similarly, untreated G3 Tert^{-/-} mice showed normal creatinine and BUN levels and normal viability at day 2. As expected, FA-treated G3 Tert^{-/-} mice showed significant increases in both BUN and creatinine levels on days 2, 7 and 14, with creatinine levels progressively increasing after day 2, indicating renal dysfunction as early as after 2 d of FA administration (FIG. 10a,b). Further biochemical parameters in the blood of Tert^{+/+} and G3 Tert^{-/-} mice indicated renal damage (Table 1). Alkaline phosphatase and amylase levels are commonly elevated in patients with CKD and those requiring dialysis30 and are indicators of renal damage3l. Impaired kidney function can result in hyperamylasemia32. At days 7 and 14, FA-treated G3 Tert^{-/-} mice showed significantly higher levels of alkaline phosphatase and amylase compared to levels in FA-treated Tert^{+/+} and untreated Tert^{+/+} mice and G3 Tert^{-/-} mice (Table 1). Hypercalcemia33, hyperphosphatemia34, hypernatremia35 and hyperkalemia36 are common complications in patients with CKD, particularly in those with end-stage renal disease. We observed increased calcium, phosphorus and sodium levels in FA-treated G3 Tert^{-/-} mice at days 2, 7 and 14, whereas potassium levels were increased only at day 14 compared to those in FA-treated Tert^{+/+} and untreated Tert^{+/+} mice and G3 Tert^{-/-} mice (Table 1). As an indicator of tubular function, we measured blood glucose, globulin, total protein and albumin levels. At days 2, 7 and 14, the blood glucose, globulin and total protein levels were significantly elevated in FA-treated G3 Tert^{-/-} mice compared to FA-treated Tert^{+/+} and untreated Tert^{+/+} mice and G3 Tert^{-/-} mice, whereas albumin remained unchanged (Table 1).

TABLE 1
Blood parameters of mice at day 2, 7 and 14 post-FA. Data are presented as mean ±
standard deviation of the mean.

| Parameters | | Tert^{+/+} | Tert^{+/+} FA | G3 Tert^{-/-} | G3 Tert^{-/-} FA |
|---|---|---|---|---|---|
| Alkaline Phosphatase | Day 2 | 107.5 ± 10.045 | 100.57 ± 9.710 | 112 ± 35.630 | 88.11 ± 17.954 |
| (U/L) | Day 7 | 112.33 ± 17.269 | 104.142 ± 13.777 | 98.6 ± 19.882^{h} | 130.44 ± 19.072^{h,i} |
| | Day 14 | 111.16 ± 15.752 | 120.142 ± 15.805 | 85.4 ± 13.240 | 150.77 ± 27.234^{c,d,h} |
| Amylase (U/L) | Day 2 | 887.33 ± 85.461 | 1022.14 ± 172.653 | 1002.2 ± 105.809 | 1012.55 ± 125.939 |
| | Day 7 | 871.5 ± 102.587 | 955.14 ± 108.490 | 998 ± 83.540 | 1229.77 ± 117.291^{a,b,i} |
| | Day 14 | 942.5 ± 108.415 | 1016.57 ± 115.412 | 1082.2 ± 78.639 | 1309.11 ± 51.074^{a,b,c} |
| Calcium(mg/dl) | Day 2 | 10.3 ± 0.163 | 10.4 ± 0.191 | 10.9 ± 0.223 | 12.06 ± 0.387^{a,b,c} |
| | Day 7 | 10.78 ± 0.291 | 10.78 ± 0.226 | 11.36 ± 0.328^{h} | 12.11 ± 0.410^{a,b,f} |
| | Day 14 | 11.35 ± 0.579 | 10.957 ± 0.732 | 11.58 ± 0.511 | 12.37 ± 0.178^{d,b} |
| Phosphorus (mg/dl) | Day 2 | 4.65 ± 0.320 | 4.67 ± 0.394 | 4.62 ± 0.383 | 6.87 ± 1.176^{a,b,c} |
| | Day 7 | 6.55 ± 0.642 | 6.41 ± 0.614 | 5.88 ± 0.697 | 7.3 ± 0.864^{h} |
| | Day 14 | 6.41 ± 0.880 | 6.68 ± 0.448 | 7.2 ± 0.703 | 8.02 ± 0.402^{a,e} |
| Sodium (mMol/L) | Day 2 | 131.33 ± 3.901 | 137.57 ± 6.347 | 133.4 ± 7.056 | 147.44 ± 4.065^{a,h} |
| | Day 7 | 146.33 ± 2.494 | 148 ± 3.958 | 145.6 ± 3.974 | 151.11 ± 1.269^{c,g} |
| | Day 14 | 146.33 ± 5.792 | 151.57 ± 7.161 | 150.6 ± 04.037 | 157.55 ± 5.364^{e} |
| Potassium(mMol/L) | Day 2 | 7.66 ± 0.460 | 8.04 ± 0.415 | 7.58 ± 0.376 | 8 ± 0.55 |
| | Day 7 | 7.8 ± 0.416 | 8.11 ± 0.338 | 7.9 ± 0.578 | 7.76 ± 0.700 |
| | Day 14 | 7.85 ± 0.309 | 8.02 ± 0.228 | 8.1 ± 0.387 | 8.42 ± 0.499^{a,e} |
| Glucose (mg/dl) | Day 2 | 118.83 ± 3.023 | 122.42 ± 5.094 | 122.2 ± 4.764 | 153 ± 14.404^{d,e} |
| | Day 7 | 136.33 ± 17.451 | 132.42 ± 28.652 | 125.2 ± 17.166 | 135.33 ± 13.793 |
| | Day 14 | 136 ± 17.635 | 114.57 ± 11.731 | 118.6 ± 11.865^{e} | 144.66 ± 9.643^{a,c,e} |
| Total Protein (g/dl) | Day 2 | 5.46 ± 0.342 | 5.5 ± 0.395 | 5.76 ± 0.270 | 6.65 ± 0.387^{a,b,f} |
| | Day 7 | 5.65 ± 0.160 | 5.67 ± 0.359 | 5.9 ± 0.070 | 6.57 ± 0.363^{a,b,j} |
| | Day 14 | 6.05 ± 0.197 | 5.71 ± 0.343 | 6.02 ± 0.258 | 6.81 ± 0.546^{e} |
| Globulin(g/dL) | Day 2 | 1.35 ± 0.15 | 1.62 ± 0.249 | 1.54 ± 0.230 | 3.02 ± 0.370^{a,b,c} |
| | Day 7 | 1.28 ± 0.146 | 1.55 ± 0.250 | 1.60 ± 0.339 | 3.21 ± 0.686^{a,b,c} |
| | Day 14 | 1.36 ± 0.110 | 1.67 ± 0.275 | 1.94 ± 0.250 | 3.7 ± 0.556^{a,b,c} |
| Albumin (g/dL) | Day 2 | 3.98 ± 0.323 | 4.12 ± 0.075 | 4.24 ± 0.151 | 3.41 ± 0.325 |
| | Day 7 | 4.35 ± 0.206 | 4.32 ± 0.179 | 4.22 ± 0.356 | 4.04 ± 0.278 |
| | Day 14 | 4.48 ± 0.240 | 4.25 ± 0.250 | 4.26 ± 0.151 | 4.23 ± 0.130 |

ªp < 0.001 vs. Tert^{+/+},
^{b}p < 0.001 vs. FA-treated Tert^{+/+},
^{c}p < 0.001 vs. G3Tert^{-/-}
^{d}p < 0.01 vs. Tert^{+/+},
^{e}p < 0.01 vs. FA-treated Tert^{+/+},
^{f}p < 0.01 vs. G3Tert^{-/-}
^{g}p < 0.05 vs. Tert^{+/+};
^{h}p < 0.05 vs. FA-treated Tert^{+/+},
ⁱp < 0.05 vs. G3Tert^{-/-}

Telomerase-Deficient Mice Show Collagen Deposition and Activated Myofibroblasts in the Kidney after a Sublethal Dose of Folic Acid

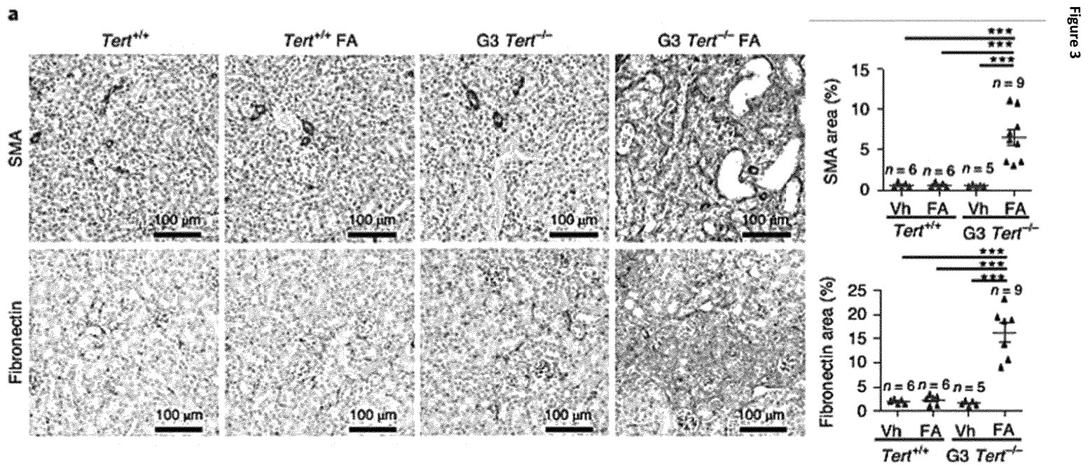

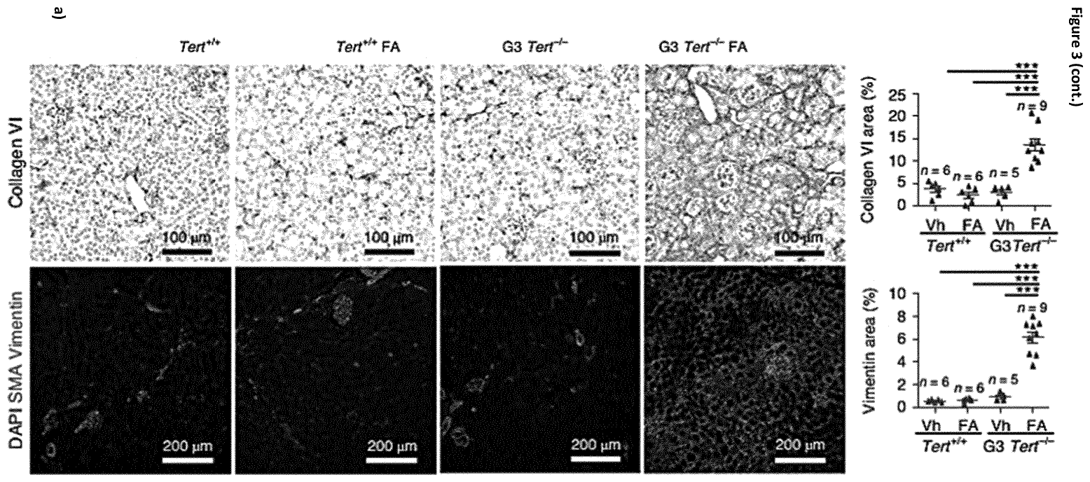

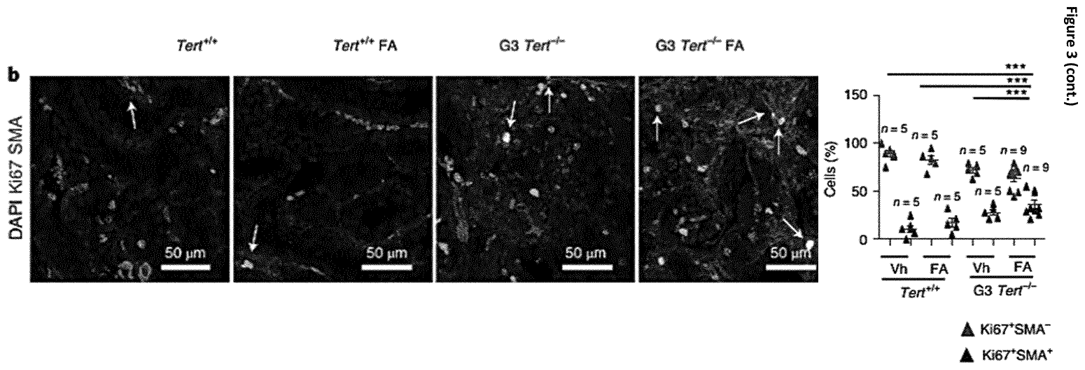

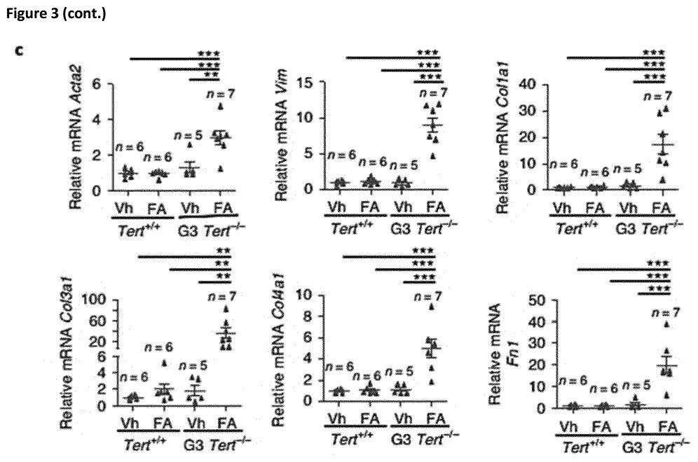

It has been previously shown that FA-induced renal injury concurs with development of segmental interstitial fibrotic lesions approximately 2 weeks after treatment37. In agreement with this, at 14 d after treatment, we observed interstitial fibrotic areas as indicated both by Masson's trichrome and Sirius red staining in the kidneys of FA-treated G3 Tert^{-/-} mice, while no fibrosis was detected in the kidneys from G3 Tert^{-/-}, Tert^{+/+} and FA-treated Tert^{+/+} mice (FIG. 2c), again indicating that short telomeres sensitize to kidney fibrosis after a subpathological dose of FA that does not induce fibrosis in wild-type mice. FA-treated G3 Tert^{-/-} mice also developed significant tubulointerstitial damage as indicated by increased PAS+D staining, including tubular dilatation, atrophy and loss of epithelial differentiation after injury, compared to normal kidney histology in the FA-treated Tert^{+/+} and untreated Tert^{+/+} mice and G3 Tert^{-/-} mice (FIG. 2c). Next, we performed immunohistochemistry for α-SMA, fibronectin and collagen type VI and double immunofluorescence with α-SMA and vimentin to detect interstitial myofibroblasts (FIG. 3a). Again, we only detected increased staining for α-SMA, fibronectin, collagen type VI and vimentin in the FA-treated G3 Tert^{-/-} mice, compared to undetectable staining in FA-treated Tert^{+/+} and untreated Tert^{+/+} mice and G3 Tert^{-/-} mice (FIG. 3a).

EMT involves a change from the apical-basolateral polarity of the epithelial cells to the front-rear polarity of the mesenchymal cells and the expression of mesenchymal markers, such as fibroblast-specific protein-1, vimentin, N-cadherin and α-SMA. These changes induce enhanced migratory capacity, invasiveness, elevated resistance to apoptosis and increased production of ECM components38. To demonstrate that the proliferating cells observed in FA-treated G3 Tert^{-/-} mice are indeed EMT cells and not tubular cells, we performed double immunofluorescence staining for Ki67 and α-SMA. We found that 36% of proliferating cells were myofibroblasts (α-SMA⁺Ki67⁺) in FA-treated G3 Tert^{-/-} mice compared to untreated G3 Tert^{-/-} mice (FIG. 3b). Increased fibrosis in the FA-treated G3 Tert^{-/-} mice but not in similarly treated wild-type or untreated mice was also confirmed by quantitative PCR (qPCR) to determine the mRNA levels of key fibrotic genes, including Acta2 (encoding α-SMA) and Vim (encoding vimentin), Col1a1, Col3a1 and Col4a1 (encoding collagen type I alpha-1, -3 and -4 chains, respectively) and Fn1 (encoding fibronectin 1). These genes were significantly upregulated in the kidneys of FA-treated G3 Tert^{-/-} mice compared to FA-treated Tert^{+/+} and untreated Tert^{+/+} mice and G3 Tert^{-/-} mice (FIG. 3c).

Increased Kidney Apoptosis and Senescence in Telomerase-Deficient Mice after a Sublethal Dose of Folic Acid

Short telomeres have been shown by us and others to induce a persistent DNA damage response at chromosome ends, leading to cell cycle arrest or apoptosis39,40. In particular, short telomeres induce the p53 and p21 cell cycle inhibitors22,41. Interestingly, p21 is known to be an activator of TGFβ37, thus providing a potential mechanism by which short telomeres, even in the absence of fibrosis, may contribute to activation of EMT programs.

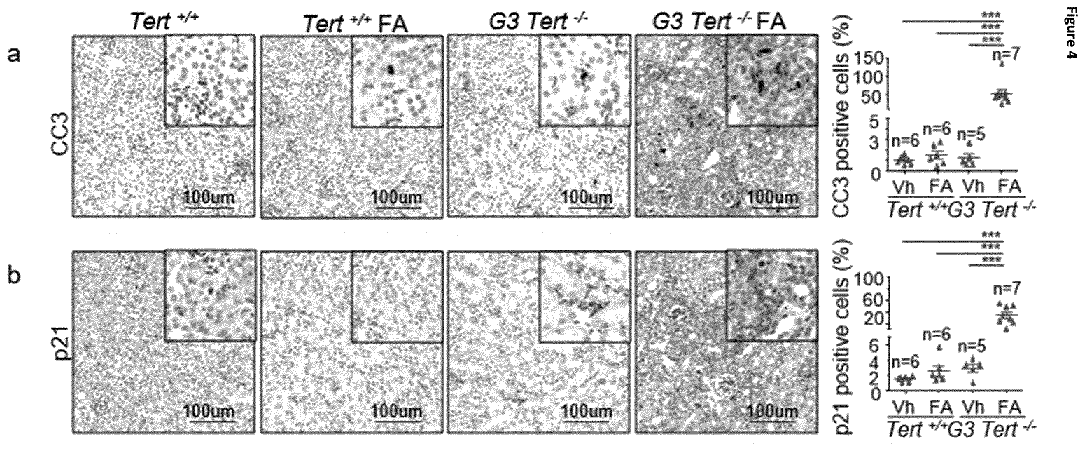

In this regard, we studied whether kidney dysfunction and increased fibrosis in G3 Tert^{-/-} mice treated with FA was accompanied by increased cellular senescence or apoptosis, two well-known cellular responses to telomere dysfunction with aging14. We observed that FA treatment induced a significant increase in levels of CC3 (apoptosis marker), p21 and p53 cell cycle inhibitors, and γ-H2AX (DNA damage marker) in G3 Tert^{-/-} mice, compared to undetectable levels in FA-treated Tert^{+/+} and untreated Tert^{+/+} mice and G3 Tert^{-/-} mice (FIG. 4a-d). Our model shows that these events produce increased turnover of kidney cells leading to telomere shortening. In support of this, we observed an increase in the transcript levels of the cell cycle regulators encoding genes CCnd1, Ccnd2, Ccnb1 and Ccne1 (FIG. 11a), suggesting that a prominent profibrotic phenotype can result in G2/M arrest. Finally, we confirmed the presence of shorter telomeres in the kidneys of G3 Tert^{-/-} mice compared to those of wild-type mice as determined by telomere quantitative fluorescence in situ hybridization (Q-FISH) directly on kidney sections (FIG. 4e). Treatment with a low dose of FA induced a clear telomere shortening in both genotypes (FIG. 4e).

### Short Telomeres Sensitize to Tubular Damage and Immune Infiltration in Kidney

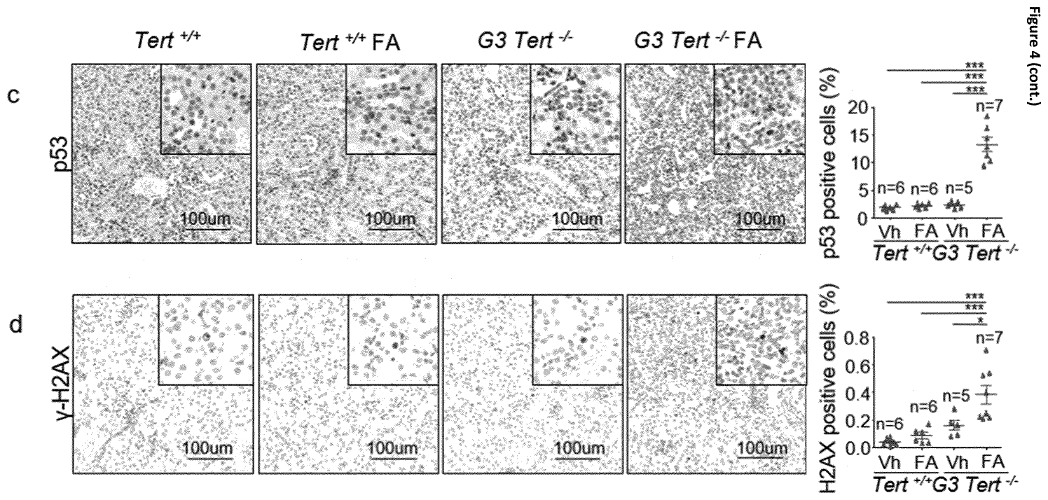

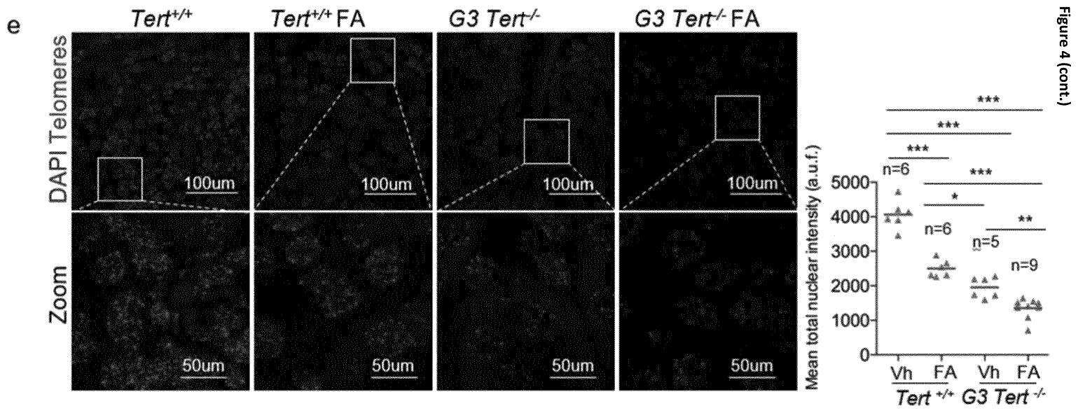

Next, we set to study the pathways by which short telomeres induce kidney injury. The kidney injury molecule-1 (KIM-1, also known as hepatitis A virus cellular receptor (HAVCR) 1; encoded by the gene Havcr1) is a type 1 transmembrane protein that is undetectable in healthy kidneys but greatly induced after injury42, localizing to the apical surface of the surviving proximal tubular cells (PTCs)43. Inflammation is proposed as a key driver of kidney fibrosis42, and the neutrophil gelatinase-associated lipocalin (NGAL) gene product (lipocalin-2 (Lcn2) or siderocalin)44 is induced during acute kidney injury (AKI)44. Thus, we used Havcr1 and Lnc2 as biomarkers of CKD due to tubulointerstitial damage. We found that Havcr1 and Lcn2 were upregulated by 134- and 206-fold, respectively (FIG. 5a) in FA-treated G3 Tert^{-/-} mice compared to basal expression in FA-treated Tert^{+/+} and control, untreated Tert^{+/+} mice and G3 Tert^{-/-} mice. Next, we studied the expression of molecules involved in immune infiltration, also associated with tissue fibrosis. We observed a 16-fold upregulation of Emr1 mRNA expression (the mouse gene encoding the F4/80 antigen) in FA-treated G3 Tert^{-/-} mice (FIG. 5a). We also examined the expression of the pan-macrophage marker F4/80 and T cell markers (CD3e, CD4 and CD8a) by immunohistochemistry. We observed increased macrophage colonization (F4/80-positive area) and increased T cells in the kidneys of FA-treated G3 Tert^{-/-} mice compared to those of FA-treated Tert^{+/+} mice or those of untreated Tert^{+/+} and G3 Tert^{-/-} mice (FIG. 5b).

### Activation of Epithelial-to-Mesenchymal Transition-Related Pathways in G3 Tert −/− Mice

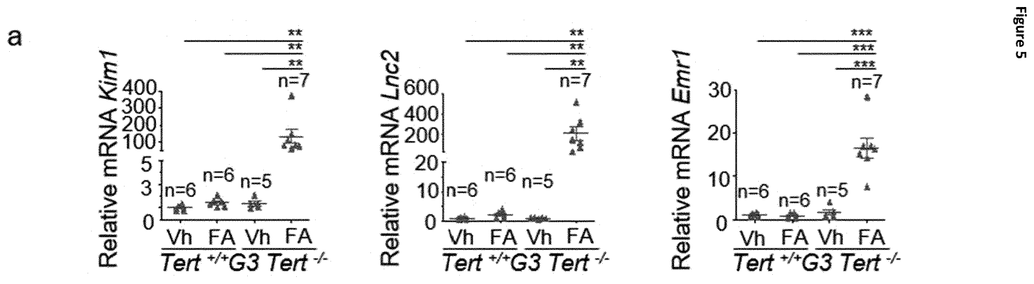

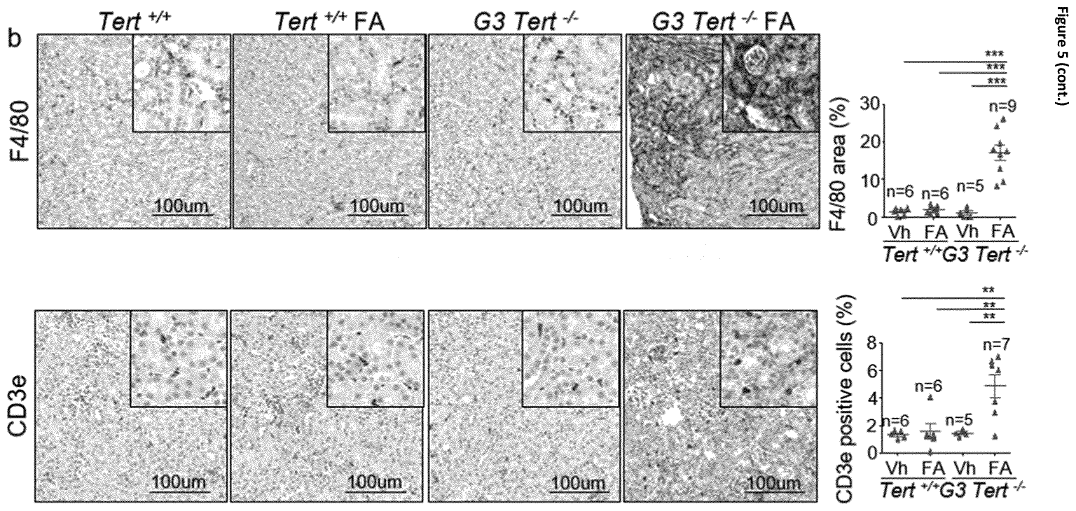

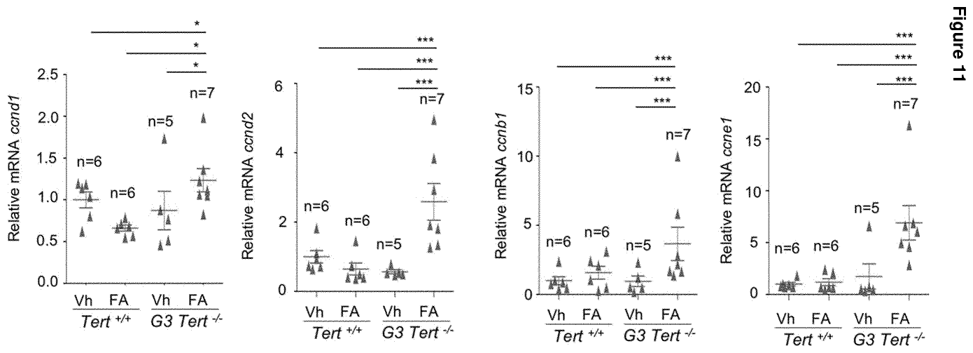

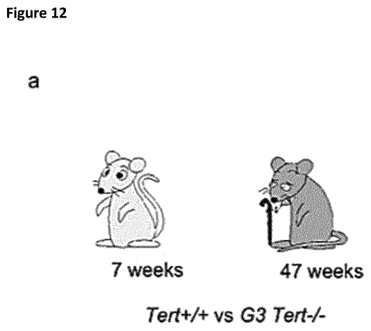

Recruitment of macrophages in tissue fibrosis is known to involve TGFβ, also important for the EMT process45 associated with kidney fibrosis46. Thus, we next sought to determine whether the presence of short telomeres is associated with changes in the expression of genes involved in EMT. To this end, we studied the expression of EMT and EMT-related pathways such as TGFβ signaling, a major environmental stimulus that induces EMT in adult epithelia, by performing RNA sequencing (RNA-seq) in the kidneys of 10-week-old Tert^{+/+} and G3 Tert^{-/-} mice either untreated or treated with low-dose FA (125 mg kg⁻¹ body weight). Gene-set enrichment analysis (GSEA) of untreated Tert^{+/+} versus untreated G3 Tert^{-/-} mice showed that the EMT (normalized enrichment score (NES)=3.37) and the TGFβ (NES=1.9) pathways were upregulated in telomerase-deficient mice compared to wild-type mice (FIG. 6), suggesting that short telomeres may contribute to a higher basal activation of some EMT genes. The transcriptional upregulation of EMT genes was more pronounced in FA-treated Tert^{+/+} and FA-treated G3 Tert^{-/-} animals (NES>6), compared to that of untreated animals. These findings were confirmed in untreated younger Tert^{+/+} versus G3 Tert^{-/-} mice at 7 weeks of age, as well as in older mice (47 weeks of age; FIG. 12a,b). Thus, short telomeres are associated with changes in the expression levels of some but not all genes involved in an EMT in kidney epithelial cells, and are therefore not sufficient to activate the classical EMT program or induce fibrosis on their own. Interestingly, treatment with FA induced an enrichment of the EMT and the TGFβ pathways in both genotypes, Tert^{+/+} and G3 Tert^{-/-}. This enrichment was higher in FA-treated G3 Tert^{-/-} kidneys compared to FA-treated Tert^{+/+} kidneys (NES=2.42), thus supporting the idea of a higher EMT activation in response to FA in telomerase-deficient mice (FIG. 6).

Upregulation of Key EMT Transcription Factors in Telomerase-Deficient Mice with Short Telomeres

TGFβ1-induced EMT is mediated by ZEB1 and SNAIL in a Smad-dependent manner45. TWIST, SNAIL and ZEB1 are transcription factors that regulate the EMT transcriptional program. Activation of Twist, Snail or Zeb1 is sufficient to induce a mesenchymal phenotype4. Thus, we set to determine the mRNA expression levels of Tgfb1, Snail1, Snail2, Twist1, Zeb1 and Zeb2 by PCR with reverse transcription (RT-PCR). We observed enhanced expression of Tgfb1 by 32-fold in FA-treated G3 Tert^{-/-} mice compared to other mouse cohorts (FIG. 6c). Snail1 and Snail2 were also upregulated by sixfold in FA-treated G3 Tert^{-/-} mice (FIG. 6c). Twist1 was upregulated by sevenfold, whereas Zeb1 and Zeb2 were upregulated by four- and five-fold, respectively, in FA-treated G3 Tert^{-/-} mice (FIG. 6c). Thus, all the major EMT players were upregulated in the kidneys of telomerase-deficient mice with short telomeres subjected to a sublethal dose of FA, compared to wild-type mice subjected to the same dose, suggesting that short telomeres may be contributing to EMT changes after FA treatment. Lysyl oxidase (LOXL2) is required and sufficient for hypoxic repression of E-cadherin, which mediates cellular transformation and induces EMT47. The expression of Lox/2 was upregulated by 20-fold (FIG. 6c) in FA-treated G3 Tert^{-/-} mice. One of the hallmarks of EMT is the downregulation of E-cadherin isoforms such as Cdh1 and expression of mesenchymal markers45. In agreement with this, the expression of the E-cadherin Cdh1 was downregulated by twofold in FA-treated G3 Tert^{-/-} mice, as determined both by mRNA levels and by immunohistochemistry, compared to the other mouse cohorts (FIG. 6c,d). SMAD3 is the key effector in TGFβ signaling. We found that Smad3 was upregulated by twofold in FA-treated G3 Tert^{-/-} mice (FIG. 6c,d).

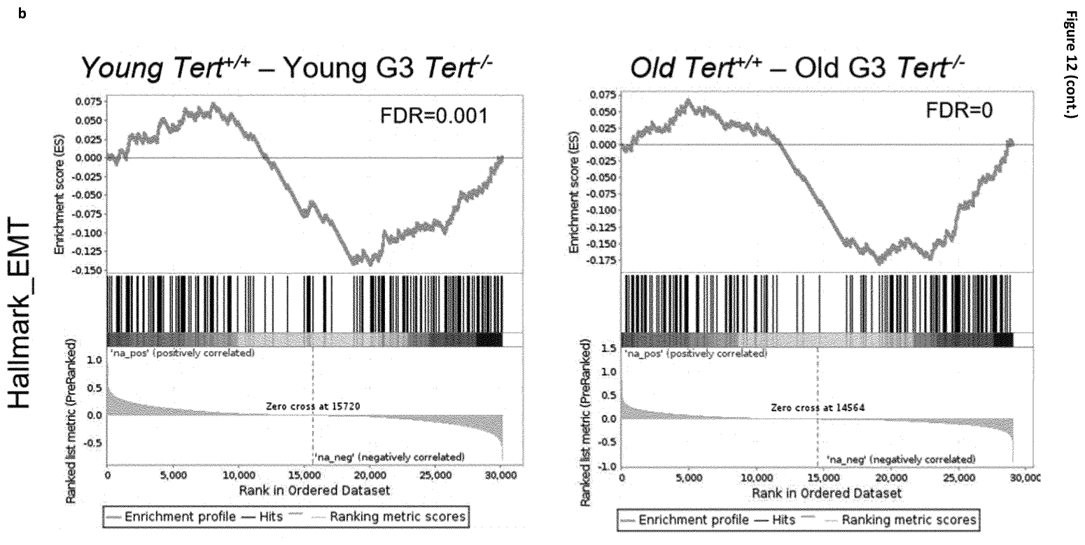

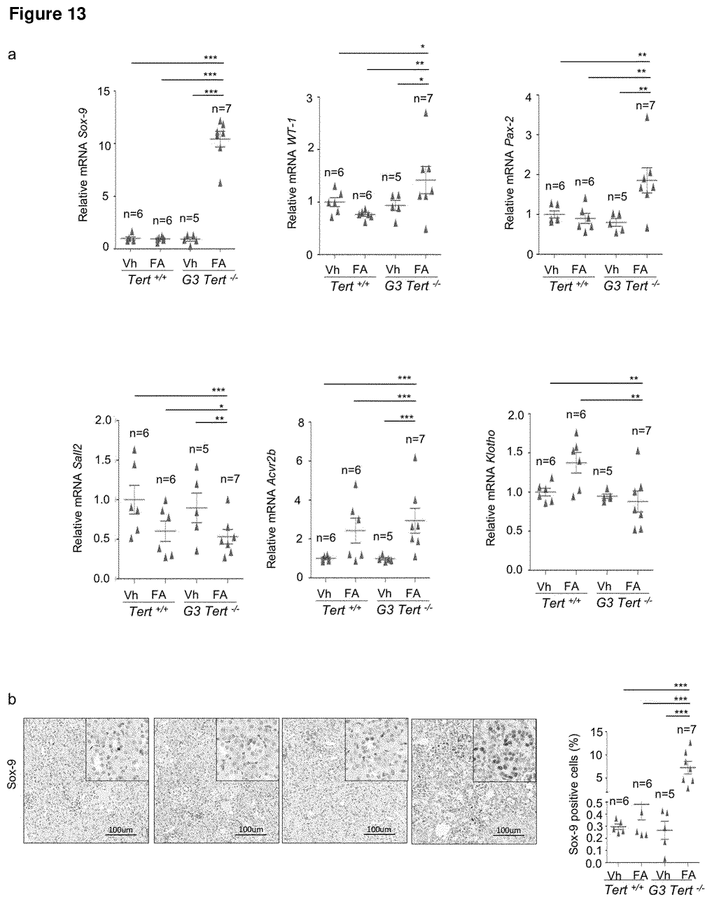

To understand how progenitor and stem cells may be affected by short telomeres during FA-induced kidney fibrosis, we studied the expression of sex determining region Y box 9 (Sox9), Wilms' tumor (Wt1), paired box 2 (Pax2), spalt like transcription factor 2 (Sall2), activin A receptor type 2B (Acvr2b, a Tgfb superfamily member) and Klotho (Kl). SOX9 is known to be important in kidney development in mouse48 and human49 studies and required for kidney fibrosis50. This precedes the expression of Wt1, Pax2 and NOTCH signaling within the epithelium. Wt1 maintains a switch between the mesenchymal and epithelial cell states and is needed to induce mesenchymal-to-epithelial transformation (MET), as well as playing a key role in the progression of nephrogenesis5l. PAX2, a nuclear transcription factor, is associated with MET and it is required for kidney cell differentiation52. Re-expression of Wt1 and Pax2 in the tubular epithelial cells plays an important role in the promotion of EMT, and there may be therapeutic value in silencing Pax2 and Wt1 to prevent or reverse kidney fibrosis53. In this regard, we observed a significant increase in the transcript levels of Sox9, Pax2, Wt1 and Acvr2b expression in Tert^{-/-} mice exposed to FA compared to all other groups, suggesting that under those conditions short telomeres sensitized the kidneys to undergo an EMT (FIG. 13a). Sox9 immunostaining was significantly increased in FA-treated G3 Tert^{-/-} mice compared to untreated and treated Tert^{+/+} mice and G3 Tert^{-/-} mice (FIG. 13b). Sall2 is negatively regulated by Wt1 (ref. 54) and Klotho (K) is an antiaging protein predominantly produced in the kidney55 that regulates telomerase activity56. A low Kl level may be a pathological intermediate for exacerbation of kidney damage. Interestingly, we found that Sall2 and Kl expression were downregulated in FA-treated G3 Tert^{-/-} mice (FIG. 13a).

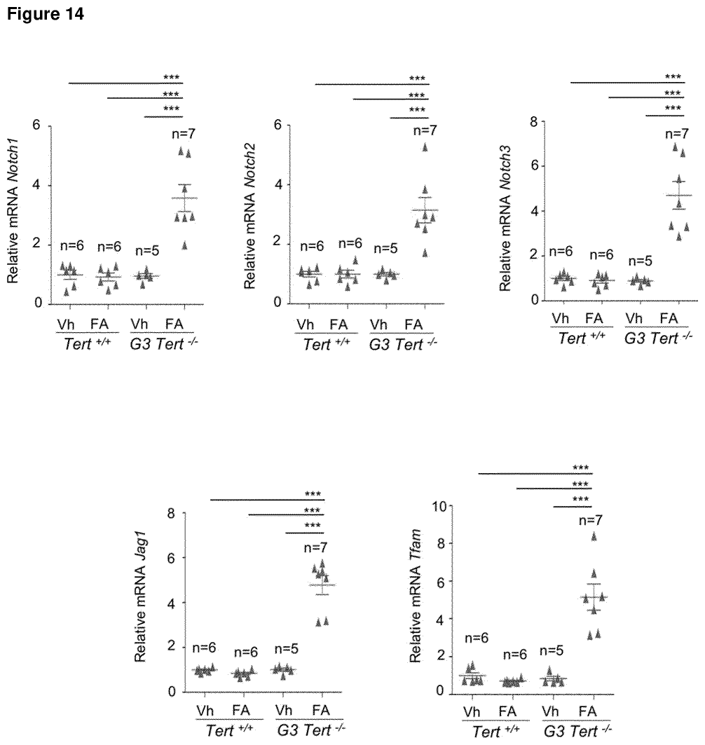

Persistent expression of NOTCH signaling pathways within the epithelium results in interstitial fibrosis57. NOTCH is a strong regulator of SNAIL1 and SNAIL2 (ref. 58). Thus, we analyzed the expression of Notch receptors (Notch1, Notch2 and Notch3), Notch ligand Jagged 1 (Jag1) and mitochondrial transcription factor A (Tfam) as a direct Notch target59 important for kidney function. We found a threefold increase in Notch1 and Notch2 and a fourfold increase in Notch3 and Jag1 in FA-treated G3 Tert^{-/-} mice compared to FA-treated Tert^{+/+} and untreated Tert^{+/+} mice and G3 Tert^{-/-} mice (FIG. 14). We also found a fivefold upregulation of Tfam in FA-treated G3 Tert^{-/-} mice compared to FA-treated Tert^{+/+} and untreated Tert^{+/+} mice and G3 Tert^{-/-} mice (FIG. 14).

Trf1 Deletion-Induced Telomere Dysfunction Triggers Kidney Fibrosis Associated with Activation of EMT

To assess the contribution of dysfunctional telomeres to the induction of kidney fibrosis, we used a second model of telomere dysfunction by deleting TRF1, one of the components of the shelterin telomere protective complex. In particular, we used a mouse model previously described by us60 in which treatment with tamoxifen led to deletion of Trf1 in all kidney cells (FIG. 7). Tamoxifen diet was maintained until the humane end point, and Trf1 deletion was confirmed by RT-qPCR (FIG. 7a). Mice deleted for Trf1 developed kidney fibrosis, as indicated by increased collagen deposition (Masson's Trichrome) and fibrotic lesions (Sirius red staining) compared to the Trf1^{+/+} mice (FIG. 7b), as well as by activation of myofibroblasts (SMA staining; FIG. 7b). We also observed a three- to four-fold increase in mesenchymal markers (Acta2 and Fn1) and a two- to six-fold increase in EMT markers (Tgfb1, Snail1, Snail2, Twist1, Zeb1 and Zeb2) in Trf1^{flox/flox} mice compared to Trf1^{+/+} mice (FIG. 7c,d), suggesting that dysfunctional telomeres may also contribute to transcriptional changes associated with EMT.

Telomerase Overexpression Rescues EMT Changes Associated with Short Telomeres

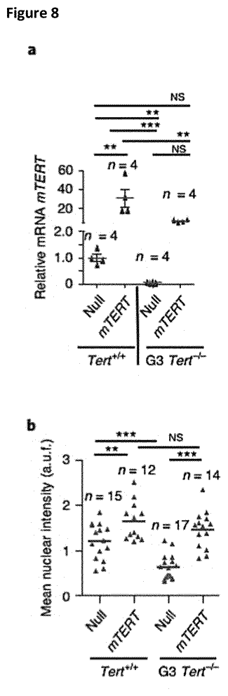

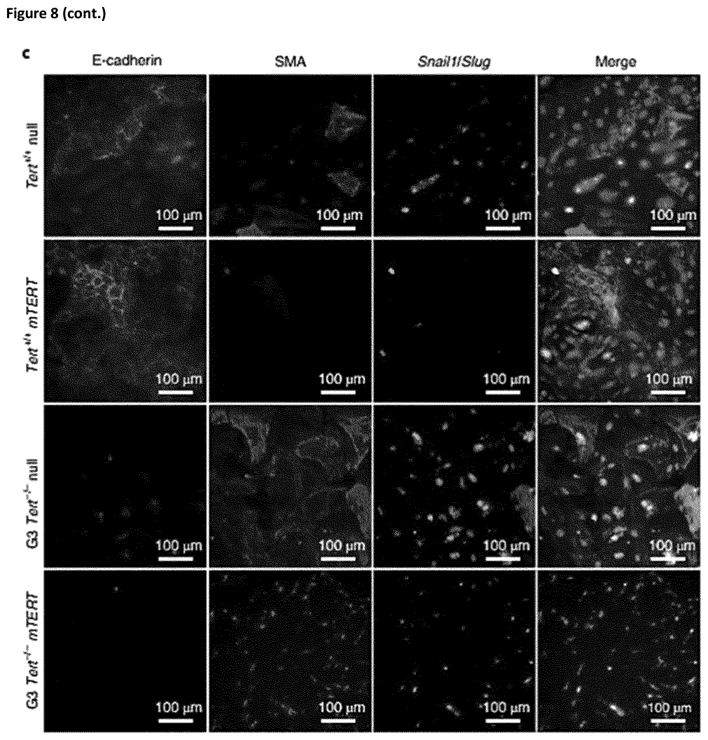

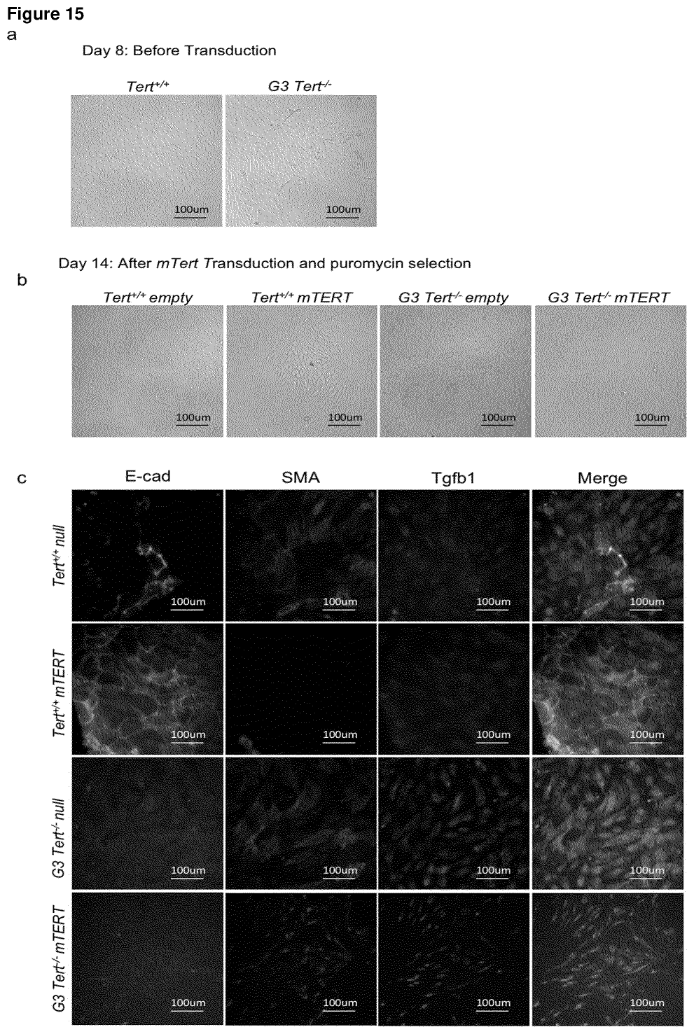

To further explore a contribution of short telomeres in the activation of EMT programs in the kidney, we addressed whether they could be rescued by expression of the catalytic subunit of telomerase or TERT61. To this end, we first isolated kidney epithelial cells, in particular PTCs, from 10- to 11-week-old Tert^{+/+} and G3 Tert^{-/-} mice. PTCs were transduced with either an empty vector or with a vector expressing Tert (Methods). Telomerase overexpression was confirmed by qPCR (FIG. 8a). Consistent with telomerase overexpression, both wild-type and G3 Tert^{-/-} cells showed a significant increase in telomere length after 1 week in culture (FIG. 8b). Before transduction, at day 8 in culture, the G3 Tert^{-/-} cells presented a clear mesenchymal morphology as indicated by a spindle-like shape that was not observed in wild-type cells, which presented a cobblestone-like morphology (FIG. 15a). G3 Tert^{-/-} cells transduced with the empty vector showed a complete myofibroblast phenotype characterized by the loss of E-cadherin, enhanced SMA staining and positivity for Snail1 and TGFβ markers (FIG. 8c and FIG. 15b,c), suggesting that 100% of the cells underwent the EMT. In contrast, control Tert^{+/+} cells transduced with the empty vector showed a mixture of epithelial cells (E-cadherin-positive, Snail1- and TGFβ-negative and SMA− negative cells) and myofibroblasts (E-cadherin-negative, Snail1- and TGFβ-positive and SMA-positive cells; FIG. 8c and FIG. 15b,c), indicating some evidence of EMT. Interestingly, Tert^{+/+} cells transduced with mTERT, showed a reverse of phenotype, including evidence of MET, restoration of the cobblestone-like phenotype in culture, expression of E-cadherin and loss of expression of SMA, TGFβ and Snail1 (FIG. 8c,d and FIG. 15b,c). Importantly, G3 Tert^{-/-} cells transduced with mTERT also showed a reverse of the mesenchymal phenotype with loss of SMA, TGFβ and Snail1 expression (FIG. 8c and FIG. 15b,c). Indeed, G3 Tert^{-/-} cells transduced with mTERT showed decreased expression of many ECM (Acta2, Vim, Co/3a1 and Co/4a1) and EMT (Tgfb1, Snail1, Snail2 and Zeb1) genes compared to G3 Tert^{-/-} cells transduced with the empty vector (FIG. 8d), confirming the restoration of the epithelial features upon TERT expression and telomere elongation. Thus, our results show that mTERT overexpression and telomere elongation is sufficient to rescue the EMT phenotype and restore the MET phenotype in kidney tubular cells. This culture system provides a potent tool to investigate EMT driven by TERT.

### Example 3: Discussion

EMT is a cellular plasticity process by which epithelial layers lose their integrity concomitant with a loss of cell polarity and cell-to-cell interactions mediated by loss of E-cadherin3. The resulting cells express mesenchymal properties, including expression of vimentin and α-SMA. EMT is orchestrated by a number of transcription factors (EMT-TFs) and has key roles both during normal development3 and in pathological conditions such as cancer and various tissue fibrotic diseases, including kidney fibrosis4,62.

As both cancer and tissue fibrosis have been associated with aging, it is of particular relevance to understand how known molecular and cellular mechanisms of aging14 may impact the expression of EMT-TFs and the origin of EMT pathological processes.

Accumulation of short and dysfunctional telomeres associated with cell division during the life span of an organism is considered one of the primary hallmarks of aging as it triggers persistent DNA damage that it is sufficient to impair the regenerative capacity of adult stem cell compartments10,13. Indeed, accelerated telomere shortening owing to telomerase deficiency both in mice24,25 and humans8 leads to premature loss of the regenerative capacity of tissues, including development of tissue fibrosis, of which pulmonary fibrosis is one of the most prevalent8,22.

Here, we generated a mouse model of fibrosis in the kidney associated with short telomeres by challenging telomerase-deficient mice with short telomeres to a low, sublethal dose of FA, a damaging agent to the kidney, which does not induce fibrosis in similarly treated wild-type controls. Telomerase-deficient mice treated with a sublethal dose of FA show all the hallmarks of the human disease including severe kidney dysfunction, as indicated by elevated blood levels of creatinine and urea. In addition, mice with dysfunctional telomeres owing to deletion of Trf1 encoding for a shelterin protein spontaneously develop kidney fibrosis, highlighting the importance of proper telomere function in the protection of fibrotic pathologies. Thus, the new mouse models generated here represent good tools to understand the role of short and dysfunctional telomeres and resulting DNA damage in the molecular events associated with fibrosis.

Interestingly, we find here that short telomeres lead to changes in the expression of genes involved in EMT, although these changes were not sufficient to induce kidney fibrosis. These changes were further exacerbated in the telomerase-deficient mice treated with FA that develop kidney fibrosis. This is supported by increased expression of Snail1, Snail2, Zeb1, Zeb2 and Twist1 transcription factors that results in epithelial cells turning into myofibroblasts. Myofibroblasts deposit ECM containing collagen leading to the development of kidney fibrosis.

Supporting evidence that short telomeres contribute to EMT changes, we show that expression of the mouse telomerase TERT catalytic subunit and the subsequent elongation of telomeres are sufficient to revert EMT programs and to restore an epithelial phenotype in kidney cells in culture.

### REFERENCES

1. Luyckx, V. A., Tonelli, M. & Stanifer, J. W. The global burden of kidney disease and the sustainable development goals. Bull. World Health Organ. 96, 414-422C (2018).
2. Humphreys, B. D. Mechanisms of renal fibrosis. Annu. Rev. Physiol. 80, 309-326 (2018).
3. Dongre, A. & Weinberg, R. A. New insights into the mechanisms of epithelial-mesenchymal transition and implications for cancer. Nat. Rev. Mol. Cell Biol. 20, 69-84 (2019).
4. Grande, M. T. et al. Snail1-induced partial epithelial-to-mesenchymal transition drives renal fibrosis in mice and can be targeted to reverse established disease. Nat. Med. 21, 989-997 (2015).
5. Inoue, T., Okada, H., Takenaka, T., Watanabe, Y. & Suzuki, H. A case report suggesting the occurrence of epithelial-mesenchymal transition in obstructive nephropathy. Clin. Exp. Nephrol. 13, 385-388 (2009).
6. Martinez, P. & Blasco, M. A. Telomere-driven diseases and telomere-targeting therapies. J. Cell Biol. 216, 875-887 (2017).
7. de Lange, T. Shelterin-mediated telomere protection. Annu. Rev. Genet. 52, 223-247 (2018).
8. Armanios, M. & Blackburn, E. H. The telomere syndromes. Nat. Rev. Genet. 13, 693-704 (2012).
9. Blackburn, E. H. Telomere states and cell fates. Nature 408, 53-56 (2000).
10. Blasco, M. A. Telomeres and human disease: ageing, cancer and beyond. Nat. Rev. Genet. 6, 611-622 (2005).
11. IJpma, A. S. & Greider, C. W. Short telomeres induce a DNA damage response in Saccharomyces cerevisiae. Mol. Biol. Cell 14, 987-1001 (2003).
12. Blasco, M. A., Funk, W., Villeponteau, B. & Greider, C. W. Functional characterization and developmental regulation of mouse telomerase RNA. Science 269, 1267-1270 (1995).
13. Flores, I., Cayuela, M. L. & Blasco, M. A. Effects of telomerase and telomere length on epidermal stem cell behavior. Science 309, 1253-1256 (2005).
14. Lopez-Otin, C., Blasco, M. A., Partridge, L., Serrano, M. & Kroemer, G. The hallmarks of aging. Cell 153, 1194-1217 (2013).
15. Armanios, M. Y. et al. Telomerase mutations in families with idiopathic pulmonary fibrosis. N. Engl. J. Med. 356, 1317-1326 (2007).
16. Armanios, M. Telomerase mutations and the pulmonary fibrosis-bone marrow failure syndrome complex. N. Engl. J. Med. 367, 384 (2012).
17. Mitchell, J. R., Wood, E. & Collins, K. A telomerase component is defective in the human disease dyskeratosis congenita. Nature 402, 551-555 (1999).
18. Tsakiri, K. D. et al. Adult-onset pulmonary fibrosis caused by mutations in telomerase.
   Proc. Natl Acad. Sci. USA 104, 7552-7557 (2007).
19. Coresh, J., Astor, B. C., Greene, T., Eknoyan, G. & Levey, A. S. Prevalence of chronic kidney disease and decreased kidney function in the adult US population: Third National Health and Nutrition Examination Survey. Am. J. Kidney Dis. 41, 1-12 (2003).
20. Beier, F., Foronda, M., Martinez, P. & Blasco, M. A. Conditional TRF1 knockout in the hematopoietic compartment leads to bone marrow failure and recapitulates clinical features of dyskeratosis congenita. Blood 120, 2990-3000 (2012).
21. Bar, C. et al. Telomerase gene therapy rescues telomere length, bone marrow aplasia and survival in mice with aplastic anemia. Blood 127, 1770-1779 (2016).
22. Povedano, J. M., Martinez, P., Flores, J. M., Mulero, F. & Blasco, M. A. Mice with pulmonary fibrosis driven by telomere dysfunction. Cell Rep. 12, 86-299 (2015).
23. Povedano, J. M. et al. Therapeutic effects of telomerase in mice with pulmonary fibrosis induced by damage to the lungs and short telomeres. eLife 7, e31299 (2018).
24. Blasco, M. A. et al. Telomere shortening and tumor formation by mouse cells lacking telomerase RNA. Cell 91, 25-34 (1997).
25. Lee, H. W. et al. Essential role of mouse telomerase in highly proliferative organs. Nature 392, 569-574 (1998).
26. Herrera, E. et al. Disease states associated with telomerase deficiency appear earlier in mice with short telomeres. EMBO J. 18, 2950-2960 (1999).
27. Bajwa, A. et al. Sphingosine kinase 2 deficiency attenuates kidney fibrosis via IFN-γ. J. Am. Soc. Nephrol. 28, 1145-1161 (2017).
28. Yang, B. et al. PHF14: an innate inhibitor against the progression of renal fibrosis following folic acid-induced kidney injury. Sci. Rep. 7, 39888 (2017).
29. Long, D. A., Woolf, A. S., Suda, T. & Yuan, H. T. Increased renal angiopoietin-1 expression in folic acid-induced nephrotoxicity in mice. J. Am. Soc. Nephrol. 12, 2721-2731 (2001).
30. Damera, S. et al. Serum alkaline phosphatase levels associate with elevated serum C-reactive protein in chronic kidney disease. Kidney Int. 79, 228-233 (2011).
31. Leibovitch, I., Ben-Chaim, J., Ramon, J. & Goldwasser, B. Increased serum alkaline phosphatase activity: a possible indicator of renal damage. J. Clin. Lab. Anal. 5, 406-409 (1991).
32. Collen, M. J., Ansher, A. F., Chapman, A. B., Mackow, R. C. & Lewis, J. H. Serum amylase in patients with renal insufficiency and renal failure. Am. J. Gastroenterol. 85, 1377-1380 (1990).
33. Moyses-Neto, M. et al. Acute renal failure and hypercalcemia. Ren. Fail. 28, 153-159 (2006).
34. Barreto, F. C., Barreto, D. V., Massy, Z. A. & Drueke, T. B. Strategies for phosphate control in patients with CKD. Kidney Int. Rep. 4, 1043-1056 (2019).
35. Cole, N. I. et al. Serum sodium concentration and the progression of established chronic kidney disease. J. Nephrol. 32, 259-264 (2019).
36. Korgaonkar, S. et al. Serum potassium and outcomes in CKD: insights from the RRI-CKD 35 cohort study. Clin. J. Am. Soc. Nephrol. 5, 762-769 (2010).
37. Yang, H.-C., Zuo, Y. & Fogo, A. B. Models of chronic kidney disease. Drug Discov. Today Dis. Models 7, 13-19 (2010).
38. Kalluri, R. & Neilson, E. G. Epithelial-mesenchymal transition and its implications for fibrosis. J. Clin. Invest. 112, 1776-1784 (2003).
39. Blasco, M. A. Telomere length, stem cells and aging. Nat. Chem. Biol. 3, 640-649 (2007).
40. d′Adda di Fagagna, F. et al. A DNA damage checkpoint response in telomere-initiated senescence. Nature 426, 194-198 (2003).
41. Gonzalez-Suarez, E., Samper, E., Flores, J. M. & Blasco, M. A. Telomerase-deficient mice with short telomeres are resistant to skin tumorigenesis. Nat. Genet. 26, 114-117 (2000).
42. Han, W. K., Bailly, V., Abichandani, R., Thadhani, R. & Bonventre, J. V. Kidney injury molecule-1 (KIM-1): a novel biomarker for human renal proximal tubule injury. Kidney Int. 62, 237-244 (2002).
43. Humphreys, B. D. et al. Chronic epithelial kidney injury molecule-1 expression causes murine kidney fibrosis. J. Clin. Invest. 123, 4023-4035 (2013).
44. Nickolas, T. L. et al. Sensitivity and specificity of a single emergency department measurement of urinary neutrophil gelatinase-associated lipocalin for diagnosing acute kidney injury. Ann. Intern. Med. 148, 810-819 (2008).
45. Stemmler, M. P., Eccles, R. L., Brabletz, S. & Brabletz, T. Non-redundant functions of EMT transcription factors. Nat. Cell Biol. 21, 102-112 (2019).
46. Lovisa, S. et al. Epithelial-to-mesenchymal transition induces cell cycle arrest and parenchymal damage in renal fibrosis. Nat. Med. 21, 998-1009 (2015).
47. Schietke, R. et al. The lysyl oxidases LOX and LOXL2 are necessary and sufficient to repress E-cadherin in hypoxia: insights into cellular transformation processes mediated by HIF-1. J. Biol. Chem. 285, 6658-6669 (2010).
48. Reginensi, A. et al. SOX9 controls epithelial branching by activating RET effector genes during kidney development. Hum. Mol. Genet. 20, 1143-1153 (2011).
49. Lindström, N. O. et al. Progressive recruitment of mesenchymal progenitors reveals a time-dependent process of cell fate acquisition in mouse and human nephrogenesis. Dev. Cell 45, 651-660 (2018).
50. Angelotti, M. L. et al. Characterization of renal progenitors committed toward tubular lineage and their regenerative potential in renal tubular injury. Stem Cells 30, 1714-1725 (2012).
51. Davies, J. A. et al. Development of an siRNA-based method for repressing specific genes in renal organ culture and its use to show that the Wt1 tumour suppressor is required for nephron differentiation. Hum. Mol. Genet. 13, 235-246 (2004).
52. Rothenpieler, U. W. & Dressler, G. R. Pax-2 is required for mesenchyme-to-epithelium conversion during kidney development. Development 119, 711-720 (1993).
53. Huang, B. et al. Wt1 and Pax2 re-expression is required for epithelial-mesenchymal transition in 5/6 nephrectomized rats and cultured kidney tubular epithelial cells. Cells Tissues Organs 195, 296-312 (2012).
54. Ma, Y. et al. Cloning and characterization of two promoters for the human HSAL2 gene and their transcriptional repression by the Wilms' tumor suppressor gene product. J. Biol. Chem. 276, 48223-48230 (2001).
55. Xu, Y. & Sun, Z. Molecular basis of Klotho: from gene to function in aging. Endocr. Rev. 36, 174-193 (2015).
56. Vera, E., Bernardes de Jesus, B., Foronda, M., Flores, J. M. & Blasco, M. A. Telomerase reverse transcriptase synergizes with calorie restriction to increase health span and extend mouse longevity. PLoS ONE 8, e53760 (2013).
57. Bielesz, B. et al. Epithelial Notch signaling regulates interstitial fibrosis development in the kidneys of mice and humans. J. Clin. Invest. 120, 4040-4054 (2010).
58. Edeling, M., Ragi, G., Huang, S., Pavenstadt, H. & Susztak, K. Developmental signalling pathways in renal fibrosis: the roles of Notch, Wnt and Hedgehog. Nat. Rev. Nephrol. 12, 426-439 (2016).
59. Huang, S. et al. Jagged1/Notch2 controls kidney fibrosis via Tfam-mediated metabolic reprogramming. PLoS Biol. 16, e2005233 (2018).
60. Bejarano, L. et al. Safety of whole-body abrogation of the TRF1 shelterin protein in wild-type and cancer-prone mouse models. iScience 19, 572-585 (2019).
61. Greider, C. W. & Blackburn, E. H. The telomere terminal transferase of etrahymena is a ribonucleoprotein enzyme with two kinds of primer specificity. Cell 51, 887-898 (1987).
62. Fazilaty, H. et al. A gene regulatory network to control EMT programs in development and disease. Nat. Commun. 10, 5115 (2019).
63. Liu, Y. et al. The telomerase reverse transcriptase is limiting and necessary for telomerase function in vivo. Curr. Biol. 10, 1459-1462 (2000).
64. Martinez, P. et al. Increased telomere fragility and fusions resulting from TRF1 deficiency lead to degenerative pathologies and increased cancer in mice. Genes Dev. 23, 2060-2075 (2009).
65. Ruzankina, Y. et al. Deletion of the developmentally essential gene ATR in adult mice leads to age-related phenotypes and stem cell loss. Cell Stem Cell 1, 113-126 (2007).
66. Terryn, S. et al. A primary culture of mouse proximal tubular cells, established on collagen-coated membranes. Am. J. Physiol. Renal Physiol. 293, F476-F485 (2007).
67. Grana, O., Rubio-Camarillo, M., Fdez-Riverola, F., Pisano, D. G. & Glez-Pena, D. Nextpresso: next-generation sequencing expression analysis pipeline. Curr. Bioinform. 13, 583-591 (2017).
68. Trapnell, C. et al. Differential gene and transcript expression analysis of RNA-seq experiments with TopHat and Cufflinks. Nat. Protoc. 7, 562-578 (2012).
69. Langmead, B., Trapnell, C., Pop, M. & Salzberg, S. L. Ultrafast and memory-efficient alignment of short DNA sequences to the human genome. Genome Biol. 10, R25 (2009).
70. Li, H. et al. The Sequence Alignment/Map format and SAMtools. Bioinformatics 25, 2078-2079 (2009).
71. Anders, S., Pyl, P. T. & Huber, W. HTSeq—a Python framework to work with high-throughput sequencing data. Bioinformatics 31, 166-169 (2015).
72. Subramanian, A. et al. Gene-set enrichment analysis: a knowledge-based approach for interpreting genome-wide expression profiles. Proc. Natl Acad. Sci. USA 102, 15545-15550 (2005).
73. Harley et al., 1990. Nature 345, 458-460; Blasco, 2007. Nat Chem Biol. 3, 640-649
74. Flores et al, 2008. Genes and Dev 22, 654-667
75. Foust et al Nature biotechnology 2009;
76. Heilbronn & Weger (2010) Handb Exp Pharmacol. 197: 143-70
77. Bernardes de Jesus et al., 2012 EMBO Mol Med. 2012 August; 4(8):691-704. doi: 10.1002/emmm.201200245.
78. Duque et al Molecular therapy 2009

1. A nucleic acid vector comprising a coding sequence for telomerase reverse transcriptase (TERT) for use in treating kidney fibrosis associated to the presence of short telomeres.

2. The nucleic acid vector for use according to claim 1, wherein TERT is encoded by a nucleic acid sequence comprising the sequence of SEQ ID NO: 1 or SEQ ID NO: 3.

3. The nucleic acid vector for use according to any of claims 1-2, wherein TERT comprises an amino acid sequence of SEQ ID NO: 2 or SEQ ID NO: 4.

4. The nucleic acid vector for use according to any of claims 1-3, wherein the nucleic acid sequence encoding TERT is operably linked to a regulatory sequence that drives the expression of the coding sequence.

5. The nucleic acid vector for use according to any of claims 1-4, wherein the vector is a non-integrative vector.

6. The nucleic acid vector for use according to any of claims 1-5, wherein the nucleic acid vector is a ribonucleic acid (RNA), preferably a messenger RNA.

7. The nucleic acid vector for use according to any of claims 1-5, wherein the vector is an adeno-associated virus-based non-integrative vector.

8. The nucleic acid vector for use according to claim 7, wherein the vector is an adeno-associated virus-based vector derived from a serotype 9 adeno-associated virus (AAV9).

9. The nucleic acid vector for use according to claim 8, wherein the capsid of the adeno-associated virus-based vector is made of capsid proteins of the serotype 9 adeno-associated virus (AAV9), and the nucleic acid sequence contained in the capsid is flanked at both ends by internal terminal repeats corresponding to serotype 2 adeno associated viruses.

10. The nucleic acid vector for use according to claim 9, wherein the nucleic acid contained in the capsid comprises a fragment which encodes the amino acid sequence coding for TERT.

11. The nucleic acid vector for use according to any of claims 1-10, wherein the vector comprises a regulatory sequence which is a constitutive promoter, preferably the cytomegalovirus (CMV) promoter.

12. A non-human animal characterized in that it exhibits a pathological condition of kidney fibrosis, wherein the non-human animal is obtained or obtainable when a sublethal dose of folic acid is administered to a non-human animal whose germ cells comprise a hereditary deactivation of both alleles of the Tert gene.

13. The non-human animal model according to claim 12, wherein the animal is a mammal, preferably a rodent.

14. The non-human animal according to any of claims 12 or 13, wherein the sublethal dose of folic acid is a dose of up to 200 mg/kg body weight, preferably of 125 mg/kg body weight.

15. The non-human animal according to any of claims 12 to 14, wherein the folic acid is administered intraperitoneally.

16. The non-human animal according to any of claims 12 to 15, wherein the folic acid is administered when the animals are between 4-10 weeks of age, preferably between 6-8 weeks of age.

17. The non-human animal according to any of claims 12 to 16, wherein the folic acid is administered once.

18. The non-human animal according to any of claims 12 to 17, wherein the non-human animal whose germ cells comprise a hereditary deactivation of both alleles of the Tert gene (Tert⁻/−) is the third generation (G3) of Tert⁻/− lineage.


SEQUENCE LISTING (49 sequences)

SEQ ID NO: 1 - DNA, 4018 bp, Homo sapiens
caggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 2 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 3 - DNA, 3982 bp, Homo sapiens
caggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 4 - protein, 1120 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 5 - DNA, 3426 bp, Mus musculus
gtgggaggcccatcccggccttgagcacaatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaaccctgtctccttccgctagatgaacatg

SEQ ID NO: 6 - DNA, 20 bp, Artificial Sequence
  Acta2 F
atcaccaactgggacgacat

SEQ ID NO: 7 - DNA, 20 bp, Artificial Sequence
  Acta2 R
agtgtcggatgctcttcagg

SEQ ID NO: 8 - DNA, 22 bp, Artificial Sequence
  Vim F
catgatgctttgggtaaaatgg

SEQ ID NO: 9 - DNA, 20 bp, Artificial Sequence
  Vim R
agtgggtgtcaaccagagga

SEQ ID NO: 10 - DNA, 22 bp, Artificial Sequence
  Col1a1 F
tggaatcatggtatttggaaca

SEQ ID NO: 11 - DNA, 20 bp, Artificial Sequence
  Col1a1 R
tagtcgtatgctgccttggg

SEQ ID NO: 12 - DNA, 19 bp, Artificial Sequence
  Col1a2 F
cagggcctgatggaaacaa

SEQ ID NO: 13 - DNA, 19 bp, Artificial Sequence
  Col1a2 R
acccctctctcctggaagc

SEQ ID NO: 14 - DNA, 20 bp, Artificial Sequence
  Col3a1 F
ataatggggaacgtggtcct

SEQ ID NO: 15 - DNA, 20 bp, Artificial Sequence
  Col3a1 R
tgaccatctgatccagggtt

SEQ ID NO: 16 - DNA, 21 bp, Artificial Sequence
  Col4a1 F
gtctggcttctgctgctcttc

SEQ ID NO: 17 - DNA, 20 bp, Artificial Sequence
  Col4a1 R
tcacattttccacagccaga

SEQ ID NO: 18 - DNA, 20 bp, Artificial Sequence
  Fn1 F
cctgggagaagtttgtgcat

SEQ ID NO: 19 - DNA, 21 bp, Artificial Sequence
  Fn1 R
acttggacaggtccagttgtg

SEQ ID NO: 20 - DNA, 20 bp, Artificial Sequence
  Emr1 F
gcaccatcttgctggagact

SEQ ID NO: 21 - DNA, 22 bp, Artificial Sequence
  Emr1 R
ttcattgatgactttgctttcg

SEQ ID NO: 22 - DNA, 20 bp, Artificial Sequence
  Tgfb1 F
ggagagccctggataccaac

SEQ ID NO: 23 - DNA, 20 bp, Artificial Sequence
  Tgfb1 R
atccacttccaacccaggtc

SEQ ID NO: 24 - DNA, 20 bp, Artificial Sequence
  Snai1 F
accctgctggtatctctccc

SEQ ID NO: 25 - DNA, 20 bp, Artificial Sequence
  Snai1 R
tcttcacatccgagtgggtt

SEQ ID NO: 26 - DNA, 20 bp, Artificial Sequence
  Snai2 F
ggctgcttcaaggacacatt

SEQ ID NO: 27 - DNA, 20 bp, Artificial Sequence
  Snai2 R
gtgccctcaggtttgatctg

SEQ ID NO: 28 - DNA, 19 bp, Artificial Sequence
  Zeb1 F
ccaagaactgctggcaaga

SEQ ID NO: 29 - DNA, 20 bp, Artificial Sequence
  Zeb1 R
ttcggatcatggttttgctc

SEQ ID NO: 30 - DNA, 20 bp, Artificial Sequence
  Zeb2 F
aaacaagccaatcccaggag

SEQ ID NO: 31 - DNA, 20 bp, Artificial Sequence
  Zeb2 R
gagggtttgcaaggctatca

SEQ ID NO: 32 - DNA, 18 bp, Artificial Sequence
  Twist F
cacgctgccctcggacaa

SEQ ID NO: 33 - DNA, 18 bp, Artificial Sequence
  Twist R
gggacgcggacatggacc

SEQ ID NO: 34 - DNA, 22 bp, Artificial Sequence
  Kim-1 F
ctctacctccactcctccaaca

SEQ ID NO: 35 - DNA, 21 bp, Artificial Sequence
  Kim-1 R
ctcctgaggatgtcacagtgc

SEQ ID NO: 36 - DNA, 20 bp, Artificial Sequence
  NGAL F
caatgtcacctccatcctgg

SEQ ID NO: 37 - DNA, 20 bp, Artificial Sequence
  NGAL R
cctggagcttggaacaaatg

SEQ ID NO: 38 - DNA, 21 bp, Artificial Sequence
  Smad3 F
tgaacaccaagtgcattacca

SEQ ID NO: 39 - DNA, 20 bp, Artificial Sequence
  Smad3 R
aggcggcagtagataacgtg

SEQ ID NO: 40 - DNA, 21 bp, Artificial Sequence
  Cdh1 F
gcagaactgtccctgtcccag

SEQ ID NO: 41 - DNA, 21 bp, Artificial Sequence
  Cdh1 R
gaacagcacgtacacagccct

SEQ ID NO: 42 - DNA, 22 bp, Artificial Sequence
  Loxl2 F
cctacaaccccaaagcctataa

SEQ ID NO: 43 - DNA, 23 bp, Artificial Sequence
  Loxl2 R
cgtgcagttcatagaaaacttcc

SEQ ID NO: 44 - DNA, 21 bp, Artificial Sequence
  GAPDH F
aggtcggtgtgaacggatttg

SEQ ID NO: 45 - DNA, 23 bp, Artificial Sequence
  GAPDH R
tgtagaccatgtagttgaggtca

SEQ ID NO: 46 - DNA, 20 bp, Artificial Sequence
  Trf1 F
tggaaaatgagaaagcgagg

SEQ ID NO: 47 - DNA, 22 bp, Artificial Sequence
  Trf1 R
tttctgtagactgctggccatt

SEQ ID NO: 48 - DNA, 21 bp, Artificial Sequence
  mTERT F
ggtgacagatgccttgaacac

SEQ ID NO: 49 - DNA, 20 bp, Artificial Sequence
  mTERT R
gatcctctccctcagacggt